SEQUENCE LISTING (47 sequences)

SEQ ID NO: 1 - DNA, 1932 bp, Kinochaeta sp
  sig_peptide (1)..(54)
atgcgtgcctcctcgattctttcgctggctctgggcctctcggttgcccaggccgctgtgaaccccgccgatgtcctgtctgttgtggagaagcgagtcgacccggctagcggcctagaggtgcgcagcatttgggacaccatctggaacgacattaaatcggcggccgactgtactgcctgcgaggccgtcttgactctgctcaagggcgtcgcggcctttggcgataattttttcgtagaggttttgaccgagatctgtgacctttccggggctgaggatgatgatgtgtgctccggtgttcttagcctcgagggcccaatcatagccaacgatatccgtaagatgagcattggctccaagacatcagagctcttctgcatcaccttcctgggactgtgctcgtacccggcggtggacgctttcaccgtccccttcccgaccgcgaagtcagccgccacccggcccgtgtcgtcgggcaaagaccccatctacgtcgtgcactactctgacatccacatcgatcccttctatgtggcaggatccgccagcaactgcaccaagcccatctgctgccgagattacacttcggcgtcgtccccgggcaacaacaactcccctgccggcccgtacggcgaccacaactgcgacgtcccgattagcctggaggacagcatgtatgctgccatcaagaagctggtgcctgatgccgccttcggcatctttactggcgatattgtcgaccacgccgtctggaatacctcggagagtcagaacatcatcgacatgaatgacgcctacacgcgcatgaagaactcgggcatgctgccgaccatcttcgccacggcgggcaaccatgaagcgtcgcccgtcaactcgttcccgccgccggccatcggcaacgagtcgcagtgggtttacgacacactggccagcgactggagccagtggatcggcacgtcgggcgcgagctcggtcgagtccatcggcgcttacagcgtgcagtacggcagcaccaagctgcgcgtcatctcgctcaacaccaacatgtactacatcgagaacttctacctctatgagcccaccatggagcaagatccagccgggcagttcgcctggctcgtgtccgagctgagcgccgccgaagccgccggcgagcgcgtgtggatcatcggccacatgccgctgggtctctcggacgccttccacgacccgagcaactactttgaccagatcgtcaaccgctacgaggccaccatcgccgccatgttcttcggccacacccacgaggaccatttccagatctcgtactcggactacaacgcccgcacggccgccaacgcccgcgccgtctcctacatcatgccgtcgctgacgccgacctcgggccacccgaccttccgcgtctacacggtcgaccccgagaccttcggcgtgctggacgcgacgacctactacgccgacatgtcgcagccgacctaccagaccgcggggccggcctggtccgtctactacagcgccaaggccgcctacggcgggctcgtcgacccgcccgtcgccgccgacgacgccgccgccgagctgacgcccgccttctggcacaacgtgacggccgcgctggccgccgacccggccagcttcgacgcctactacgcgcgcaagacgcgcggctgggacgtggccgcctgcgccggcgcctgcgcggccgccgaggtctgcgccctgcgcgccgcccgcgcccaggacaactgcgtcgtgcccacgcccggcgtgcacttcagcaagcgcgccgacgagggcaccctggcccaccaccgcgacgagtgcggcgtcagcgtcgcccgcaacagcctctccagcctcgtcgtgcagcgcgaggcgctggagcacctcgagggccgcctgagcgagaagcggaggatggccgtgtga

SEQ ID NO: 2 - protein, 643 aa, Kinochaeta sp
MRASSILSLALGLSVAQAAVNPADVLSVVEKRVDPASGLEVRSIWDTIWNDIKSAADCTACEAVLTLLKGVAAFGDNFFVEVLTEICDLSGAEDDDVCSGVLSLEGPIIANDIRKMSIGSKTSELFCITFLGLCSYPAVDAFTVPFPTAKSAATRPVSSGKDPIYVVHYSDIHIDPFYVAGSASNCTKPICCRDYTSASSPGNNNSPAGPYGDHNCDVPISLEDSMYAAIKKLVPDAAFGIFTGDIVDHAVWNTSESQNIIDMNDAYTRMKNSGMLPTIFATAGNHEASPVNSFPPPAIGNESQWVYDTLASDWSQWIGTSGASSVESIGAYSVQYGSTKLRVISLNTNMYYIENFYLYEPTMEQDPAGQFAWLVSELSAAEAAGERVWIIGHMPLGLSDAFHDPSNYFDQIVNRYEATIAAMFFGHTHEDHFQISYSDYNARTAANARAVSYIMPSLTPTSGHPTFRVYTVDPETFGVLDATTYYADMSQPTYQTAGPAWSVYYSAKAAYGGLVDPPVAADDAAAELTPAFWHNVTAALAADPASFDAYYARKTRGWDVAACAGACAAAEVCALRAARAQDNCVVPTPGVHFSKRADEGTLAHHRDECGVSVARNSLSSLVVQREALEHLEGRLSEKRRMAV

SEQ ID NO: 3 - DNA, 1833 bp, Penicillium emersonii
  sig_peptide (1)..(48)
atgagagttctcgccctcatcgctgctctggccacggtggccaccgcaagtgccccctatgacaagcgcgacttggcccaggagatttgggacgacatcaagaatgcggtggattgcgctggctgccaggtcgttctgactgccctgaagggtgtggccgatctgggcacgactgcccttgtcgatgtgctgaccgaagtgtgcaacatcagtggcaaagaagattccgatgtctgctcgggcatcatctcccgcgagggtccggtgctggattatgtcctgcagcacctcgatatcggctcgcacacctcccaggtcatctgtgccagcgcattcggcctctgccagtatcctgaggtccggccctacaacctcaccttccctaaacccaagcccaacacgactcgtccagaacccagtggagagtcaccaatccaggtcgtccacttcagcgatactcacgtggacctctcctacgagacggggtccaattacaactgtacaaagcccatctgctgtcgcccttacacggccgaggatgcaccgggaaacacgacgactccgtgcgggccatatggcaacaccaaatgtgatgctcccttgagcctcgaggagagcatgttcgccgcgatcaaagcgctcaacccccagcccgccttttccatttatacgggcgacgtcgtcgcacacgacatctggctggtggatcaaaacgaggtcattgaggacctgaatgccacctacgaccgcatggccgggctggggctggtctatgcggccattgggaatcacgacacggcgccggtcaacgatctgccgacgagcaacatccccagcgagtacagcgcgaactggacctacgaggccctctcgtacgactttacgatgctgacgcagtcggcctctgcccagaccgcggcgaattacgggtcttattcggccatctatcccggcagctacggcacggatctccgcgtcatttcctacaacagcatcttctactacgtggacaatttctgggcgtaccaagatcctatggaattcgacccggatggacaactggcctggctgatcaacgagctccaggaggccgagacggcggggcagcgggtctggattattgcgcatgtgccgacgggcacgtcggatcacttccacgactattcgcactactttgaccagatcgtgcagcgctacgaggccactattgcggcgctgttctacggccacactcacatcgaccagttccaaatctcgtactcgaactattccaaccgagcattcgacacggcgaccgccatcgggtatatcatgccgtcattgactccgacctcgggacctcctaccttccgggtctatgacgttgatcccaagacgtttgccgtgctggacttcaccaactacattgccaacatcagcgacccggcgttccagtcgggcccgtcgtggcagaagtactactcggccaaggagacgtacggctcgttgctgtctcctccagtgacggacccgacggcggagctgacgccggccttctggcacaacgtcacggtggcctttgagcaggacaacgcgaccttccaggagtactgggcgcggcagacgcgggggtacgacgtgtcgagctgcacggggtcctgcatcactcaggccatctgcggcctgcgcgcgggagacgcgcagtacaactgcgtgacgccgacgccgggcttcaactttgccaaacgggatacctccaaccccaagcaggctctatctcatgtcgagaaatgcgagggctcgggattgctggggctgctgcgcaggatggtggctgacagtaagtcttcctag

SEQ ID NO: 4 - protein, 610 aa, Penicillium emersonii
MRVLALIAALATVATASAPYDKRDLAQEIWDDIKNAVDCAGCQVVLTALKGVADLGTTALVDVLTEVCNISGKEDSDVCSGIISREGPVLDYVLQHLDIGSHTSQVICASAFGLCQYPEVRPYNLTFPKPKPNTTRPEPSGESPIQVVHFSDTHVDLSYETGSNYNCTKPICCRPYTAEDAPGNTTTPCGPYGNTKCDAPLSLEESMFAAIKALNPQPAFSIYTGDVVAHDIWLVDQNEVIEDLNATYDRMAGLGLVYAAIGNHDTAPVNDLPTSNIPSEYSANWTYEALSYDFTMLTQSASAQTAANYGSYSAIYPGSYGTDLRVISYNSIFYYVDNFWAYQDPMEFDPDGQLAWLINELQEAETAGQRVWIIAHVPTGTSDHFHDYSHYFDQIVQRYEATIAALFYGHTHIDQFQISYSNYSNRAFDTATAIGYIMPSLTPTSGPPTFRVYDVDPKTFAVLDFTNYIANISDPAFQSGPSWQKYYSAKETYGSLLSPPVTDPTAELTPAFWHNVTVAFEQDNATFQEYWARQTRGYDVSSCTGSCITQAICGLRAGDAQYNCVTPTPGFNFAKRDTSNPKQALSHVEKCEGSGLLGLLRRMVADSKSS

SEQ ID NO: 5 - DNA, 2115 bp, Nectria mariannaeae
  sig_peptide (1)..(57)
  Intron (169)..(222)
  Intron (310)..(360)
  Intron (666)..(727)
  Intron (937)..(985)
atgcaattactctctatcctcgccgtcgggctcggcctcgcgcagaacgcattctgccaagaggtcactcatgatcttgccggtatcaagcgatccctagaatccagagattgggttgaggatctttgggataagttcgaaagtgatgcaacatgcgcgggatgcgaggttagccattcatgttgatttggtgttgagcatgacgagctaatgaaatattagtcactcgtgttagtgctcaagggcttggccgctataagtgatcaagcgtttattgacgtccttcaggagatttgcaagatctccggggtgagtacaccttgactatggtgcttctaagtggagttgacgatgcagtaggctgaggatgacgatgtttgtgacggttcaattcaacttgaaggccccgttattgccagcggtcttcgatcaatggctatcggctctcgaacttccaaagaattctgtactacattccttggactttgcgcgtaccctgcggtgcagcaatggagtgttcccttctcgtcctccaagtcttccaagactcgtccttcgtccagtggaaaggaccctatcaaggtggttcattattctgatattcatatcgatcctttatatgtcgggggctcaaactccaactgcactaagcccatatgttgtaggtaagagataaccccaaacacttgccagcaaaactggacatgttgataactaaactacgaaggtcatacaccaaggctgaccagcccgggaacaacaaatatcctgctggaccgaacggcgaccataactgcgattctccagtcagtcttgagaagagcatgtacaacgccatcaaggaaatcgttccagatgcagccttcaccatcttcaccggggacattgtcgatcatgcagtctggaatactagccaatcctataatacggaacaaagtgtgttcagtataatccatccagtggaataacttactaaatctcacagttaccaatgcctacggcttgatgagtgataatctgggcacaatctatgggactgccggcaaccatgaagctcatcccgctaatgccttccaacctaactccgtcggcaacgtgagccaatgggtatacgatcttctttcaggtctctggtcacagtggattagcaccgaagccaaagctgactcggagaagcttggcgcttactctaccaagtatcctggcggcaacttgcgcattatctctctcaacaccaacatgtactatcgagaaaactactggctctaccgcaagacgatgattcaggaccctagcaatcagatttcctggctcgtaaacgaactcgaagccgctgagactgcgggcgagcgcgtttatatcatcggccacatgccgcttggagactctaacagttttcacgaccagtccaactaccttgaccaagtaatcaaccgctactccgcaactatctctgccatgttctttggccacacacacgacgaccagttccagataagttactccaattggtcaaatcgcaacttctccaatgccctcgtaacctcctacattggtccctcactcactcccaccgccggtatgcccgcctttcgcgtttacgatgtcgaccccgtgacgttcggaatcctcgactcgaccacatacatcgctgacatgaccgactctgcctttcaaaccactggcccagtgtggaagaagtattattctgccaaagaagtttacggctctttattgagcccggctgtgactgacagcagcgccgagctcacggcagccttctggcacaacgtgacgaccctgttcgaggcggacaacaccgcatttgaggcgtttctttcccgcaagagccgtgggtggaagtcagaatcctgcacaggcacttgcaaggccaacgagatctgtcaattgcgcgcggctcgcagcgagaacaattgctacaccccgtcgctaggaattagcttcaacaaacgaagtctgaacccagttgaagagcgggacgagtgtgggatttcagtgactagggctacggttagtgctatgggcgtaaggaaagacgttttgcgtttgttaaagaagaggtttatcgagaaggcgggtgaggtccgtggctga

SEQ ID NO: 6 - DNA, 1899 bp, Artificial sequence
  cDNA
atgcaattactctctatcctcgccgtcgggctcggcctcgcgcagaacgcattctgccaagaggtcactcatgatcttgccggtatcaagcgatccctagaatccagagattgggttgaggatctttgggataagttcgaaagtgatgcaacatgcgcgggatgcgagtcactcgtgttagtgctcaagggcttggccgctataagtgatcaagcgtttattgacgtccttcaggagatttgcaagatctccggggctgaggatgacgatgtttgtgacggttcaattcaacttgaaggccccgttattgccagcggtcttcgatcaatggctatcggctctcgaacttccaaagaattctgtactacattccttggactttgcgcgtaccctgcggtgcagcaatggagtgttcccttctcgtcctccaagtcttccaagactcgtccttcgtccagtggaaaggaccctatcaaggtggttcattattctgatattcatatcgatcctttatatgtcgggggctcaaactccaactgcactaagcccatatgttgtaggtcatacaccaaggctgaccagcccgggaacaacaaatatcctgctggaccgaacggcgaccataactgcgattctccagtcagtcttgagaagagcatgtacaacgccatcaaggaaatcgttccagatgcagccttcaccatcttcaccggggacattgtcgatcatgcagtctggaatactagccaatcctataatacggaacaaattaccaatgcctacggcttgatgagtgataatctgggcacaatctatgggactgccggcaaccatgaagctcatcccgctaatgccttccaacctaactccgtcggcaacgtgagccaatgggtatacgatcttctttcaggtctctggtcacagtggattagcaccgaagccaaagctgactcggagaagcttggcgcttactctaccaagtatcctggcggcaacttgcgcattatctctctcaacaccaacatgtactatcgagaaaactactggctctaccgcaagacgatgattcaggaccctagcaatcagatttcctggctcgtaaacgaactcgaagccgctgagactgcgggcgagcgcgtttatatcatcggccacatgccgcttggagactctaacagttttcacgaccagtccaactaccttgaccaagtaatcaaccgctactccgcaactatctctgccatgttctttggccacacacacgacgaccagttccagataagttactccaattggtcaaatcgcaacttctccaatgccctcgtaacctcctacattggtccctcactcactcccaccgccggtatgcccgcctttcgcgtttacgatgtcgaccccgtgacgttcggaatcctcgactcgaccacatacatcgctgacatgaccgactctgcctttcaaaccactggcccagtgtggaagaagtattattctgccaaagaagtttacggctctttattgagcccggctgtgactgacagcagcgccgagctcacggcagccttctggcacaacgtgacgaccctgttcgaggcggacaacaccgcatttgaggcgtttctttcccgcaagagccgtgggtggaagtcagaatcctgcacaggcacttgcaaggccaacgagatctgtcaattgcgcgcggctcgcagcgagaacaattgctacaccccgtcgctaggaattagcttcaacaaacgaagtctgaacccagttgaagagcgggacgagtgtgggatttcagtgactagggctacggttagtgctatgggcgtaaggaaagacgttttgcgtttgttaaagaagaggtttatcgagaaggcgggtgaggtccgtggctga

SEQ ID NO: 7 - protein, 632 aa, Nectria mariannaeae
  SIGNAL (1)..(19)
  PROPEP (20)..(36)
MQLLSILAVGLGLAQNAFCQEVTHDLAGIKRSLESRDWVEDLWDKFESDATCAGCESLVLVLKGLAAISDQAFIDVLQEICKISGAEDDDVCDGSIQLEGPVIASGLRSMAIGSRTSKEFCTTFLGLCAYPAVQQWSVPFSSSKSSKTRPSSSGKDPIKVVHYSDIHIDPLYVGGSNSNCTKPICCRSYTKADQPGNNKYPAGPNGDHNCDSPVSLEKSMYNAIKEIVPDAAFTIFTGDIVDHAVWNTSQSYNTEQITNAYGLMSDNLGTIYGTAGNHEAHPANAFQPNSVGNVSQWVYDLLSGLWSQWISTEAKADSEKLGAYSTKYPGGNLRIISLNTNMYYRENYWLYRKTMIQDPSNQISWLVNELEAAETAGERVYIIGHMPLGDSNSFHDQSNYLDQVINRYSATISAMFFGHTHDDQFQISYSNWSNRNFSNALVTSYIGPSLTPTAGMPAFRVYDVDPVTFGILDSTTYIADMTDSAFQTTGPVWKKYYSAKEVYGSLLSPAVTDSSAELTAAFWHNVTTLFEADNTAFEAFLSRKSRGWKSESCTGTCKANEICQLRAARSENNCYTPSLGISFNKRSLNPVEERDECGISVTRATVSAMGVRKDVLRLLKKRFIEKAGEVRG

SEQ ID NO: 8 - DNA, 969 bp, pseudomonas sp
  sig_peptide (1)..(75)
atgactcatctcattggtttcgcccctcggttgctggctttttcggcgctgttgctcagccagacggcattcagccaggaaagcccggcattcatcgatcctgcgtcgtggaacaccccgttcaatggcattgcccaggtggcttgtcataactgctacgagaagcaatacgcgaacaccttcagcagtgtgcttgacagtgtacggaccctggagctggacttctgggaccagcgcgatgcggtgagcggcggttcgccccatcactggttcgtgcggcacaaccccggcaccttgttccaatccggcaacgacaataactgcaccggcgatggcaccggcaagaacgacctcgaagcctgtctgaacgacgtcaagaactggagtgacaagcatccggggcacttccccatcacgctgatcctggacaagaaacagggctggtcgaaagaaagttcggggcgcacaccaaaggatttcgacgaactggtggcgcgggtgttccagggcaagctctttaccccccaggatctggcgacgcacattggcagtggcgcgggggccttgcagggcaacctcaagggtaagtcctggcccaccgccaacgatctgcagggcaaggtgctgttggtgctcaaccactcggaaaaccagaagctctcgcagtacgccgaggcccgcacctctaaggctaaggtgttcatttcgccagtaaccaacggccagaacgatatcagtggcaaggtcagcggcatgtccagccagtcatccggctatgtagccatgaacaacatgggcaagggcgacaaaagttgggcaaagcaggcctttgcctacagccatatcggccgcgtctggggtgatgacgaggtgtcgttcgcccagcacatcaaccagaagatcaatctgtcggcgtactacaggttcgccgcgcagagcgctggcggctaccgcatccggccgttctga

SEQ ID NO: 9 - protein, 322 aa, pseudomonas sp
  SIGNAL (1)..(25)
MTHLIGFAPRLLAFSALLLSQTAFSQESPAFIDPASWNTPFNGIAQVACHNCYEKQYANTFSSVLDSVRTLELDFWDQRDAVSGGSPHHWFVRHNPGTLFQSGNDNNCTGDGTGKNDLEACLNDVKNWSDKHPGHFPITLILDKKQGWSKESSGRTPKDFDELVARVFQGKLFTPQDLATHIGSGAGALQGNLKGKSWPTANDLQGKVLLVLNHSENQKLSQYAEARTSKAKVFISPVTNGQNDISGKVSGMSSQSSGYVAMNNMGKGDKSWAKQAFAYSHIGRVWGDDEVSFAQHINQKINLSAYYRFAAQSAGGYRIRPF

SEQ ID NO: 10 - protein, 298 aa, Artificial sequence
  Variant
AQESPAFIDPASWNTPFNGIAQVACHNCYEKQYANTFSSVLDSVRTLELDFWDQRDAVSGGSPHHWFVRHNPGTLFQSGNDNNCTGDGTGKNDLEACLNDVKNWSDKHPGHFPITLILDKKQGWSKESSGRTPKDFDELVARVFQGKLFTPQDLATHIGSGAGALQGNLKGKSWPTANDLQGKVLLVLNHSENQKLSQYAEARTSKAKVFISPVTNGQNDISGKVSGMSSQSSGYVAMNNMGKGDKSWAKQAFAYSHIGRVWGDDEVSFAQHINQKINLSAYYRFAAQSAGGYRIRPF

SEQ ID NO: 11 - DNA, 972 bp, Pseudomonas chlororaphis
  sig_peptide (1)..(75)
atgtcccgtctcactcgctttgccctccgtaccgccgtgatgcccctggtgctggccagccagatggcgtccagccaggaggctgtgggattcatttccccggcttcctggtacaccgccttcaacgctatcgcccaggtggcgtgccataactgctacgaaaaacagtacgccggcaccttcaccagcgtgctcgacagcgtgcgcaccctggagctggacttctgggaccaacgcgatgcggtcaccggaggttcgccccgccactggttcgtgcggcacaaccccggcagcctgttccagtccggcaatgacaacaattgcaccggcgacggcaaaggcaccaacgatcttgaggcctgcctcaatgacatcaagctctggagcgacagccatcccgggcacttcccgattaccctgatcctcgacaagaagcagggctggtcgaaggaaagctccgggcgtacgcctaaagacttcgatgacctggtcagccggattttccagggcaagctctacaccccgggcgacctggcccagcacctgggtgtcagcagcagtgccttgcagggctcgctcaagggcaagtcctggccgaccgccagccaactgcagggcaaggtgctgttggtgctcaaccactcggagaaccagaagctctcgcaatacgccgaggcccgcaccaccagcgccaaggtgttcatttccccggtcaccaacggccagaacgatgtcagtggcgaggtcagcggcatgtccaggacgtcgtccggctacgtggccatgaacaacatgggcaagggcgacaagcagtgggccgcgcaagcctttgcctacagccacatcggtcgggtgtggggcgatgacggcgtgtcattcacccagcacatcgccgagaaggtcaatctgtcggcgtattacaaattcgccgaggccaaggatggcaacggctatcgcatccggccgttctga

SEQ ID NO: 12 - protein, 323 aa, pseudomonas chlororaphis
  SIGNAL (1)..(25)
MSRLTRFALRTAVMPLVLASQMASSQEAVGFISPASWYTAFNAIAQVACHNCYEKQYAGTFTSVLDSVRTLELDFWDQRDAVTGGSPRHWFVRHNPGSLFQSGNDNNCTGDGKGTNDLEACLNDIKLWSDSHPGHFPITLILDKKQGWSKESSGRTPKDFDDLVSRIFQGKLYTPGDLAQHLGVSSSALQGSLKGKSWPTASQLQGKVLLVLNHSENQKLSQYAEARTTSAKVFISPVTNGQNDVSGEVSGMSRTSSGYVAMNNMGKGDKQWAAQAFAYSHIGRVWGDDGVSFTQHIAEKVNLSAYYKFAEAKDGNGYRIRPF

SEQ ID NO: 13 - protein, 299 aa, Artificial sequence
  Variant
AQEAVGFISPASWYTAFNAIAQVACHNCYEKQYAGTFTSVLDSVRTLELDFWDQRDAVTGGSPRHWFVRHNPGSLFQSGNDNNCTGDGKGTNDLEACLNDIKLWSDSHPGHFPITLILDKKQGWSKESSGRTPKDFDDLVSRIFQGKLYTPGDLAQHLGVSSSALQGSLKGKSWPTASQLQGKVLLVLNHSENQKLSQYAEARTTSAKVFISPVTNGQNDVSGEVSGMSRTSSGYVAMNNMGKGDKQWAAQAFAYSHIGRVWGDDGVSFTQHIAEKVNLSAYYKFAEAKDGNGYRIRPF

SEQ ID NO: 14 - DNA, 972 bp, pseudomonas sp
  sig_peptide (1)..(75)
atgtcccgtctcactggttttgccctccgtaccgccgtgctgcccctggcactggccagccagatggcgtccagccaggaggccgtgggattcatttccccggcgtcctggtacaccgccttcaacgctatcgcccaggtggcatgccataactgctacgaaaaacagtacgccggcaccttcaccagcgtgctcgatagcgtgcgcaccctggagctggacttctgggaccaacgcgatgcggtcaccggaggttcgccccgccactggttcgtgcggcacaaccccggcagcctgttccagtccggcaatgacaacaattgcaccggcgacggcaacggcaccaacgatctcgaagcctgcctcaacgacatcaagctctggagcgacagccatcccgggcacttcccgattaccctgatcctcgacaagaagcagggctggtcgaaggaaagctccgggcgtacgccgaaagacttcgatgacctggtcagccggattttccagggcaagctctacaccccgggcgacctggcccagcatctgggtgtcagcagcagtgccttgcagggctcgctcaagggcaagtcctggccgaccgccagccaactgcagggcaaggtgctgctggtgctcaaccactcggagaaccagaagctctcgcaatacgccgaggcccgcaccaccagcgccaaggtgttcatttccccggtcaccaacggccagaacgatgtcagtggcgaggtcagcggcatgtcgaggacgtcgtccggctacgtggccatgaacaacatgggcaagggcgacaagcagtgggccgcgcaagcctttgtctacagccacatcggtcgggtgtggggtgatgacggcgtgtcattcacccagcacatcgccgagaaggtcaatctgtcggcgtattacaaattcgccgaggccaaggatggcaacggctatcgcatccgaccgttctga

SEQ ID NO: 15 - protein, 323 aa, pseudomonas sp
  SIGNAL (1)..(25)
MSRLTGFALRTAVLPLALASQMASSQEAVGFISPASWYTAFNAIAQVACHNCYEKQYAGTFTSVLDSVRTLELDFWDQRDAVTGGSPRHWFVRHNPGSLFQSGNDNNCTGDGNGTNDLEACLNDIKLWSDSHPGHFPITLILDKKQGWSKESSGRTPKDFDDLVSRIFQGKLYTPGDLAQHLGVSSSALQGSLKGKSWPTASQLQGKVLLVLNHSENQKLSQYAEARTTSAKVFISPVTNGQNDVSGEVSGMSRTSSGYVAMNNMGKGDKQWAAQAFVYSHIGRVWGDDGVSFTQHIAEKVNLSAYYKFAEAKDGNGYRIRPF

SEQ ID NO: 16 - protein, 299 aa, Artificial sequence
  Variant
AQEAVGFISPASWYTAFNAIAQVACHNCYEKQYAGTFTSVLDSVRTLELDFWDQRDAVTGGSPRHWFVRHNPGSLFQSGNDNNCTGDGNGTNDLEACLNDIKLWSDSHPGHFPITLILDKKQGWSKESSGRTPKDFDDLVSRIFQGKLYTPGDLAQHLGVSSSALQGSLKGKSWPTASQLQGKVLLVLNHSENQKLSQYAEARTTSAKVFISPVTNGQNDVSGEVSGMSRTSSGYVAMNNMGKGDKQWAAQAFVYSHIGRVWGDDGVSFTQHIAEKVNLSAYYKFAEAKDGNGYRIRPF

SEQ ID NO: 17 - DNA, 972 bp, Pseudomonas chlororaphis
  sig_peptide (1)..(84)
atgtcccgtctcactcgttttgccctccgtacagccgtactgcccctggcgctggccagccagatggcgtccagccaggaggccgcgggattcatttctccggcttcctggtacagcgccttcaacgctatcgcccaggtggcgtgccataactgctacgaaaaacagtacgccagcaccttcaccagtgtgctcgacagcgtgcgtaccctggagctggatttctgggaccagcgcgatgcggtcaccggtggttcggccggtcactggttcgtgcggcacaaccccggcacgctgttccagtccggcaatgacaacaactgcaccggcgacggcaaaggcaccaacgatctcgaggcctgcctcaacgacatcaggctctggagcgacagccatcccgggcacttcccgattaccctgatcctcgacaagaagcagggctggtcgaaggaaagctccgggcgtacgccgaaagacttcgatgatctggtcagccggattttccagggcaagctctataccccgggcgacctggcccagcacctgggtgtcagcagcggtgccttgcagggctcgctcgagggcaagtcctggccgaccgctagccaactgcagggcaaggtgctgctggtgctcaaccactcggagaaccagaagctctcgcaatacgccgaagcccggaccaccagcgccaaggtgttcatttcaccggtcaccaacggccagaacgatgtcagtggcgaggtcagcggcatgtccaggacgtcgtccggttacgtggccatgaacaacatgggcaagggcgacaagcagtgggccgcacaagcctttgcctacagccacatcggtcgggtgtggggtgatgacggcgtgtcattcacccagcacatcgccgagaaggtcaatctgtcggcgtattacaagttcgccgaagccaaggatggcaacggctatcgcatccggccgttctaa

SEQ ID NO: 18 - protein, 323 aa, Pseudomonas chlororaphis
  SIGNAL (1)..(28)
MSRLTRFALRTAVLPLALASQMASSQEAAGFISPASWYSAFNAIAQVACHNCYEKQYASTFTSVLDSVRTLELDFWDQRDAVTGGSAGHWFVRHNPGTLFQSGNDNNCTGDGKGTNDLEACLNDIRLWSDSHPGHFPITLILDKKQGWSKESSGRTPKDFDDLVSRIFQGKLYTPGDLAQHLGVSSGALQGSLEGKSWPTASQLQGKVLLVLNHSENQKLSQYAEARTTSAKVFISPVTNGQNDVSGEVSGMSRTSSGYVAMNNMGKGDKQWAAQAFAYSHIGRVWGDDGVSFTQHIAEKVNLSAYYKFAEAKDGNGYRIRPF

SEQ ID NO: 19 - protein, 296 aa, Artificial sequence
  Variant
AAGFISPASWYSAFNAIAQVACHNCYEKQYASTFTSVLDSVRTLELDFWDQRDAVTGGSAGHWFVRHNPGTLFQSGNDNNCTGDGKGTNDLEACLNDIRLWSDSHPGHFPITLILDKKQGWSKESSGRTPKDFDDLVSRIFQGKLYTPGDLAQHLGVSSGALQGSLEGKSWPTASQLQGKVLLVLNHSENQKLSQYAEARTTSAKVFISPVTNGQNDVSGEVSGMSRTSSGYVAMNNMGKGDKQWAAQAFAYSHIGRVWGDDGVSFTQHIAEKVNLSAYYKFAEAKDGNGYRIRPF

SEQ ID NO: 20 - DNA, 969 bp, Pseudomonas protegens
  sig_peptide (1)..(75)
atgactcatctttcccgtttcgtcccccgcgccctggcgctgtcggcactcttgctcagcccggcggcgttcagccaggaaagcccggcgttcatcgccccggctacctggaataccccgttcaacggcatcgcccaggtggcctgtcacaactgctacgagaagcaatacgcaagcaccttcagcagcgtgctcgacagtgtgcgcaccctggaactggacttctgggaccagcgcgatgcggtgaccggcggctcgccccggcactggttcgtgcggcacaaccccggcaccctgttccagtccggcaacgacaacaattgcacgggcgatggcaccggcaagaacgatctcgaagcctgcctcaacgacgtcaagaactggagcgaaaaccaccccgggcactttcccatcacggtgatcctcgacaagaagcagggctggtcgaaggaaagctcggggcgcacgcccaaggatttcgatgagctggtgacccgggtcttccagggcaagctctataccccccaggacctggccacgcacatcggcagcagcgcgggcgccttgcagggcaacctcaagggcaagtcctggcccaccgccagccagcttcagggcaaggtcctgctggtgctcaaccactcggaaaaccagaagctctcgcagtacgccgaggcccggacttccagcgccaaggtgttcatctcgccggtgaccaatggccagaacgatgtcagcggcaaggtcagtggcatgtccggccagtcgtccggctacgtggccatgaacaacatgtccaagggcgacaagaagtgggcggcccaggcctttgcctacagccatgtgggccgggtctggggcgatgacggggtgtcgttcgcccagcacatcagcgagaaggtcaacctctcggcctactacaagttcgccgagcagaactccggcggctatcgcatccggccgttctga

SEQ ID NO: 21 - protein, 322 aa, Pseudomonas protegens
  SIGNAL (1)..(25)
MTHLSRFVPRALALSALLLSPAAFSQESPAFIAPATWNTPFNGIAQVACHNCYEKQYASTFSSVLDSVRTLELDFWDQRDAVTGGSPRHWFVRHNPGTLFQSGNDNNCTGDGTGKNDLEACLNDVKNWSENHPGHFPITVILDKKQGWSKESSGRTPKDFDELVTRVFQGKLYTPQDLATHIGSSAGALQGNLKGKSWPTASQLQGKVLLVLNHSENQKLSQYAEARTSSAKVFISPVTNGQNDVSGKVSGMSGQSSGYVAMNNMSKGDKKWAAQAFAYSHVGRVWGDDGVSFAQHISEKVNLSAYYKFAEQNSGGYRIRPF

SEQ ID NO: 22 - protein, 298 aa, Artificial sequence
  Variant
AQESPAFIAPATWNTPFNGIAQVACHNCYEKQYASTFSSVLDSVRTLELDFWDQRDAVTGGSPRHWFVRHNPGTLFQSGNDNNCTGDGTGKNDLEACLNDVKNWSENHPGHFPITVILDKKQGWSKESSGRTPKDFDELVTRVFQGKLYTPQDLATHIGSSAGALQGNLKGKSWPTASQLQGKVLLVLNHSENQKLSQYAEARTSSAKVFISPVTNGQNDVSGKVSGMSGQSSGYVAMNNMSKGDKKWAAQAFAYSHVGRVWGDDGVSFAQHISEKVNLSAYYKFAEQNSGGYRIRPF

SEQ ID NO: 23 - DNA, 969 bp, Pseudomonas protegens
  sig_peptide (1)..(75)
atgactcatctttcccgtttcgtcccccgcgccctggcgctgtcggcactcctgctcagcccggcggcgttcagccaggaaagcccggcgttcatcgccccggctacctggaataccccgttcaacggcatcgcccaggtggcctgtcacaactgctacgagaagcagtacgcaagcacctttagcagcgtgctcgacagtgtgcgcaccctggaactggacttctgggaccagcgcgatgcggtgaccggcggctcgccccggcactggttcgtgcggcacaaccccggcaccctgttccagtccggcaacgacaacaattgcacgggcgatggcaccggcaagaacgatctcgaagcctgcctcaacgacgtgaagaactggagcgacaaccaccccgggcactttcccattacggtgatcctcgacaagaagcagggctggtcgaaggaaagctcggggcgcacgcccaaggatttcgatgagctggtgacccgggtcttccagggcaagctctataccccccaggacctggccacgcacatcggcagcagcgcgggcgccttgcagggcaacctcaagggcaagtcctggcccaccgccagccagcttcagggcaaggtcctgctggtgctcaaccactcggaaaaccagaagctctcgcagtacgccgaggcccggacttccagcgccaaggtgttcatctcgccggtgaccaatggccagaacgatgtcagtggcaaggtcagtggcatgtccggccagtcgtccggctacgtggccatgaacaacatgtccaagggcgacaagaagtgggcggcccaggcctttgcctacagccatgtgggccgggtctggggcgatgacggggtgtcgttcgcccagcacatcagcgagaaggtcaacctctcggcctactacaagttcgccgagcagaacgccggcggctatcgcatccggccgttctga

SEQ ID NO: 24 - protein, 322 aa, Pseudomonas protegens
  SIGNAL (1)..(25)
MTHLSRFVPRALALSALLLSPAAFSQESPAFIAPATWNTPFNGIAQVACHNCYEKQYASTFSSVLDSVRTLELDFWDQRDAVTGGSPRHWFVRHNPGTLFQSGNDNNCTGDGTGKNDLEACLNDVKNWSDNHPGHFPITVILDKKQGWSKESSGRTPKDFDELVTRVFQGKLYTPQDLATHIGSSAGALQGNLKGKSWPTASQLQGKVLLVLNHSENQKLSQYAEARTSSAKVFISPVTNGQNDVSGKVSGMSGQSSGYVAMNNMSKGDKKWAAQAFAYSHVGRVWGDDGVSFAQHISEKVNLSAYYKFAEQNAGGYRIRPF

SEQ ID NO: 25 - DNA, 837 bp, Bacillus sp
  sig_peptide (1)..(99)
atgaaacatcatcgttttcgaacgaatttattatcggctctttcagtaagttcaattgtcatcacctcaatcatcggctccactcaaaccacgtacgcttggtcggccgatgctccgcatgatcccaatcagagcacacacctgtttatcgtgaacggtgctgtcaatctggttgccaacaatacggacccgcagatcaacaagcccaccgcgctgttgcaacagtggcgttctcaatgggagcaagggttgtacgatgccgatcatctgaacccttactatgactccggcacttttatgtcccatttttacgatccggacacgcaaacgaactacgcagggttgtcgtacccgacagcacgccaaacaggggcaaaatattttacgattgcctcgaatgactatcaagcaggggatatgtcggatgcattttacaatttaggcttgtccctgcactatttcacagatgtcacgatgccgcttcatgccggaaatatttccaatctcgatcacgaagcacccggctaccatgcgaaactcgaagcgtatgccgaatcgattcaaaatcaagtgacgcctccgactgccggactctacaattgggtctctccgaatgatccggaactctggattcatcaagcggctgtgcaagcgaaatccgtcttgccgcaagtatggaacagtgatatcacgagttggttctgggaggcggctttcagcaattactactcgcaacaatggcacaacgcagtaaccactccggtcctgaatcagttgtcgcaagcggaggcagagaccgccggatatattgatctgttcttccgtgtaaacggttag

SEQ ID NO: 26 - protein, 278 aa, Bacillus sp
  SIGNAL (1)..(33)
MKHHRFRTNLLSALSVSSIVITSIIGSTQTTYAWSADAPHDPNQSTHLFIVNGAVNLVANNTDPQINKPTALLQQWRSQWEQGLYDADHLNPYYDSGTFMSHFYDPDTQTNYAGLSYPTARQTGAKYFTIASNDYQAGDMSDAFYNLGLSLHYFTDVTMPLHAGNISNLDHEAPGYHAKLEAYAESIQNQVTPPTAGLYNWVSPNDPELWIHQAAVQAKSVLPQVWNSDITSWFWEAAFSNYYSQQWHNAVTTPVLNQLSQAEAETAGYIDLFFRVNG

SEQ ID NO: 27 - protein, 246 aa, Artificial sequence
  Variant
AWSADAPHDPNQSTHLFIVNGAVNLVANNTDPQINKPTALLQQWRSQWEQGLYDADHLNPYYDSGTFMSHFYDPDTQTNYAGLSYPTARQTGAKYFTIASNDYQAGDMSDAFYNLGLSLHYFTDVTMPLHAGNISNLDHEAPGYHAKLEAYAESIQNQVTPPTAGLYNWVSPNDPELWIHQAAVQAKSVLPQVWNSDITSWFWEAAFSNYYSQQWHNAVTTPVLNQLSQAEAETAGYIDLFFRVNG

SEQ ID NO: 28 - DNA, 852 bp, Bacillus thuringiensis
  sig_peptide (1)..(72)
atgaaaaagaaagtacttgctttagcagcagctattacattagtagctcctttacaaagcgttgcatttgctcatgaaaatgatgggggaagtaaaataaaaatagttcaccgctggtctgctgaagataaacataaagaaggcgtaaattctcatttatggattgtaaaccgtgcgattgatattatgtctcgcaatacaacacttgtaaaacaagatcgagttgcacaattaaatgaatggcgtacagagttagagaacggtatttatgctgctgactatgaaaatccttattatgataatagcacatttgcttcacatttctatgatccagacaatggaaaaacatatattccatttgcaaagcaggcaaaagaaactggagctaaatattttaaattagcgggtgaatcatacaaaaataaagatatgaaacaagcattcttctatttaggattatctcttcattatttaggagatgtaaatcaaccgatgcatgcggcaaactttacaaatctttcgtatccacaaggattccattctaaatatgaaaactttgtagatacgataaaagataattataaagtaacggatggaaatggatattggaattggaaaggtacaaatccagaagattggatccatggagcggcagtagtggcgaaacaagattactctggaattgtaaatgataatacgaaagattggttcgtaaaagcagctgtgtcacaagaatatgcagataaatggcgtgctgaagttacaccgatgacaggtaagcgattaatggatgcacaacgtgttactgctggatacattcagctttggtttgatacgtacggagatcgttaa

SEQ ID NO: 29 - protein, 283 aa, Bacillus thuringiensis
  SIGNAL (1)..(24)
  PROPEP (25)..(38)
MKKKVLALAAAITLVAPLQSVAFAHENDGGSKIKIVHRWSAEDKHKEGVNSHLWIVNRAIDIMSRNTTLVKQDRVAQLNEWRTELENGIYAADYENPYYDNSTFASHFYDPDNGKTYIPFAKQAKETGAKYFKLAGESYKNKDMKQAFFYLGLSLHYLGDVNQPMHAANFTNLSYPQGFHSKYENFVDTIKDNYKVTDGNGYWNWKGTNPEDWIHGAAVVAKQDYSGIVNDNTKDWFVKAAVSQEYADKWRAEVTPMTGKRLMDAQRVTAGYIQLWFDTYGDR

SEQ ID NO: 30 - DNA, 852 bp, Bacillus pseudomycoides
  sig_peptide (1)..(72)
atgaaaaagaaagtattagccttagcagcagctattacattagtagcaccattgcaaagtgtagcgtttgcccatgaaaatgagggcggaaataaggtaagagtaattcaatattggtctgctgaagataaacatgcagaaggtgtaaactcccatttatggattgtcaatcgtgcaattgatattatgtctcgtaatacaacggttgtaaaacaagatcaagttgcagtattaaatgaatggcgtacagagttagagaatggtatatatgctgctgattatgaaaacccttactatgataacagtacatttgcttctcatttctacgagcctgatacagggaagacatatataccttttgctaagcaggcaaaagaaactggggctaaatattttaaacttgctggtgaagcttatcaaaagcaagatatgaaacaagcattcttctatttgggtttatcccttcattacttaggcgatgtcaatcaaccgatgcatgcagcaaactttacaaatctttcttatccacaaggtttccactccaaatatgaaaattttgtagatacaataaaaaataattataaagtggctgatggaaatggatattggaattggaaaggagtaaatcctgaagactggattcatggagcggctgtagctgctaagcaagattatgctggtattgtaaatggcactacaaaagattggttcgtaagagcggcagtttcacaagaatatgcagataaatggcgtgcagaagttacactaacaacaggaaaacgtttagtagaagcacagcgtgtcacagcgggatatattcagctttggtttgatacgtatgtaaatcgctag

SEQ ID NO: 31 - protein, 283 aa, Bacillus pseudomycoides
  SIGNAL (1)..(24)
  PROPEP (25)..(38)
MKKKVLALAAAITLVAPLQSVAFAHENEGGNKVRVIQYWSAEDKHAEGVNSHLWIVNRAIDIMSRNTTVVKQDQVAVLNEWRTELENGIYAADYENPYYDNSTFASHFYEPDTGKTYIPFAKQAKETGAKYFKLAGEAYQKQDMKQAFFYLGLSLHYLGDVNQPMHAANFTNLSYPQGFHSKYENFVDTIKNNYKVADGNGYWNWKGVNPEDWIHGAAVAAKQDYAGIVNGTTKDWFVRAAVSQEYADKWRAEVTLTTGKRLVEAQRVTAGYIQLWFDTYVNR

SEQ ID NO: 32 - protein, 260 aa, Artificial sequence
  Variant
AHENEGGNKVRVIQYWSAEDKHAEGVNSHLWIVNRAIDIMSRNTTVVKQDQVAVLNEWRTELENGIYAADYENPYYDNSTFASHFYEPDTGKTYIPFAKQAKETGAKYFKLAGEAYQKQDMKQAFFYLGLSLHYLGDVNQPMHAANFTNLSYPQGFHSKYENFVDTIKNNYKVADGNGYWNWKGVNPEDWIHGAAVAAKQDYAGIVNGTTKDWFVRAAVSQEYADKWRAEVTLTTGKRLVEAQRVTAGYIQLWFDTYVNR

SEQ ID NO: 33 - DNA, 852 bp, Bacillus mycoides
  sig_peptide (1)..(72)
atgaaaaagaaagtattagccttagcagcagctattacattagtagcaccattgcaaagtgtagcgtttgcccatgaaaatgagggcggaaataaggtaagagtaattcaatattggtctgctgaagataaacatgcagaaggtgtaaactcccatttatggattgtcaatcgtgcaattgatattatgtctcgtaatacaacggttgtaaaacaagatcaagttgcattattaaatgaatggcgtacagagttagagaatggtatatatgctgctgattatgaaaacccttattatgataacagtacatttgcttctcatttctacgatcctgacacagggaagacatatataccttttgctaagcaggcaaaagaaactggggctaaatattttaaacttgctggtgaagcctatcaaaagcaagaaataaaacaagcattcttctatttaggcttatcgcttcattacttaggagatgtaaatcaaccaatgcatgcagcaaactttacaaatctttcttatccacaaggtttccactccaaatatgaaaattttgtagatacaataaaaaataattataaagtggctgatggaaatggatattggaattggaaaggagtaaatcctgaagactggattcatggagcggctgtagctgctaagcaagattatgctggtattgtaaatggcactacaaaagattggttcgtaagagcggcagtttcacaagaatatgcagataaatggcgtgcagaagttacactaacaacaggaaaacgtttagtagaagcacagcgtgtcacagcgggatatattcagctttggtttgatacatatgtaaatcgctag

SEQ ID NO: 34 - protein, 283 aa, Bacillus mycoides
  SIGNAL (1)..(24)
MKKKVLALAAAITLVAPLQSVAFAHENEGGNKVRVIQYWSAEDKHAEGVNSHLWIVNRAIDIMSRNTTVVKQDQVALLNEWRTELENGIYAADYENPYYDNSTFASHFYDPDTGKTYIPFAKQAKETGAKYFKLAGEAYQKQEIKQAFFYLGLSLHYLGDVNQPMHAANFTNLSYPQGFHSKYENFVDTIKNNYKVADGNGYWNWKGVNPEDWIHGAAVAAKQDYAGIVNGTTKDWFVRAAVSQEYADKWRAEVTLTTGKRLVEAQRVTAGYIQLWFDTYVNR

SEQ ID NO: 35 - DNA, 870 bp, Listeria innocua
  sig_peptide (1)..(81)
atgaaattcaaaaaggtagttctaggtatgtgtttgactgcaagtgttctagtctttccggtaacgataaaagcaagtgcctgttgcgatgaatatttacaagcacccgcagctccgcatgatatcgacagtaaattaccacataaacttagttggtccgcggataacccgacaaatactgacgtaaatacacactattggctttttaaacaagctgaaaaaatactagctaaagatgtaaatcatatacgagctaatttaatgaatgagcttaaaaatttcgataaacaaattgctcaaggtatatatgatgcggatcataaaaatccatattatgatactagtacatttttatctcatttttataatcctgatagagataatacttatttgccgggttttgctaatgcgaaaataactggagccaagtatttcaatcaatcggtggctgattatcgagaaggtaaatttgacacagcattttataaattaggtctagcaatccattattatacggatattagtcaacctatgcacgccaataattttaccgcaatatcatacccaccaggctaccactgcgcatatgaaaattatgtggatactattaaacacaattatcaagcaacagaagacatggtagtgaaaagattttgctcagatgacgtgaaagtctggctctatgaaaatgcgaaaagagcaaaagccgactaccctaaaatagtcaatgcaaaaactaaaaaatcatatttagtaggaaattccaaatggaaaaaggatacagtggaacctactggagctagactaagagattcacagcaaactttggcaggctttttagaattttggtccaaaaaaacaaatgaataa

SEQ ID NO: 36 - protein, 289 aa, Listeria innocua
  SIGNAL (1)..(27)
MKFKKVVLGMCLTASVLVFPVTIKASACCDEYLQAPAAPHDIDSKLPHKLSWSADNPTNTDVNTHYWLFKQAEKILAKDVNHIRANLMNELKNFDKQIAQGIYDADHKNPYYDTSTFLSHFYNPDRDNTYLPGFANAKITGAKYFNQSVADYREGKFDTAFYKLGLAIHYYTDISQPMHANNFTAISYPPGYHCAYENYVDTIKHNYQATEDMVVKRFCSDDVKVWLYENAKRAKADYPKIVNAKTKKSYLVGNSKWKKDTVEPTGARLRDSQQTLAGFLEFWSKKTNE

SEQ ID NO: 37 - protein, 263 aa, Artificial sequence
  Variant
ACCDEYLQAPAAPHDIDSKLPHKLSWSADNPTNTDVNTHYWLFKQAEKILAKDVNHIRANLMNELKNFDKQIAQGIYDADHKNPYYDTSTFLSHFYNPDRDNTYLPGFANAKITGAKYFNQSVADYREGKFDTAFYKLGLAIHYYTDISQPMHANNFTAISYPPGYHCAYENYVDTIKHNYQATEDMVVKRFCSDDVKVWLYENAKRAKADYPKIVNAKTKKSYLVGNSKWKKDTVEPTGARLRDSQQTLAGFLEFWSKKTNE

SEQ ID NO: 38 - DNA, 849 bp, Artificial sequence
  Variant
atgaaaaagaaagtattagcactagcagctatggttgctttagctgcgccagttcaaagtgtagtatttgcacaaacaaataatagtgaaagtcctgcaccgattttaagatggtcagctgaggataagcataatgaggggattaactctcatttgtggattgtaaatcgtgcaattgacatcatgtctcgtaatacaacgattgtgaatccgaatgaaactgcattattaaatgagtggcgtgctgatttagaaaatggtatttattctgctgattacgagaatccttattatgatgatagtacatatgcttctcacttttatgatccggatactggaacaacatatattccttttgcgaaacatgcaaaagaaacaggcgcaaaatattttaaccttgctggtcaagcataccaaaatcaagatatgcagcaagcattcttctacttaggattatcgcttcattatttaggagatgtgaatcagccaatgcatgcagcatcttttacggatctttcttatccaatgggtttccattctaaatacgaaaattttgttgatacaataaaaaataactatattgtttcagatagcaatggatattggaattggaaaggagcaaacccagaagattggattgaaggagcagcggtagcagctaaacaagattatcctggcgttgtgaacgatacgacaaaagattggtttgtaaaagcagccgtatctcaagaatatgcagataaatggcgtgcggaagtaacaccggtgacaggaaagcgtttaatggaagcgcagcgcgttacagctggttatattcatttgtggtttgatacgtatgtaaatcgctaa

SEQ ID NO: 39 - protein, 282 aa, Artificial sequence
  Variant
MKKKVLALAAMVALAAPVQSVVFAQTNNSESPAPILRWSAEDKHNEGINSHLWIVNRAIDIMSRNTTIVNPNETALLNEWRADLENGIYSADYENPYYDDSTYASHFYDPDTGTTYIPFAKHAKETGAKYFNLAGQAYQNQDMQQAFFYLGLSLHYLGDVNQPMHAASFTDLSYPMGFHSKYENFVDTIKNNYIVSDSNGYWNWKGANPEDWIEGAAVAAKQDYPGVVNDTTKDWFVKAAVSQEYADKWRAEVTPVTGKRLMEAQRVTAGYIHLWFDTYVNR

SEQ ID NO: 40 - DNA, 987 bp, Unknown
  DNA obtained from an environmental sample
atgaacaataagaagtttattttgaagttattcatatgtagtatggtacttagcgcctttgtatttgctttcaatgataagaaaaccgttgcagctagctctattaatgtgcttgaaaattggtctagatggatgaaacctataaatgatgacataccgttagcacgaatttcaattccaggaacacatgatagtggaacgttcaagttgcaaaatccgataaagcaagtgtggggaatgacgcaagaatatgattttcgttatcaaatggatcatggagctagaatttttgatataagagggcgtttaacagatgataatacgatagttcttcatcatgggccattatatctttatgtaacactgcacgaatttataaacgaagcgaaacaatttttaaaagataatccaagtgaaacgattattatgtctttaaaaaaagagtatgaggatatgaaaggggcggaaagctcatttagtagtacgtttgagaaaaattattttcgtgatccaatctttttaaaaacagaagggaatataaagcttggagatgctcgtgggaaaattgtattactaaaaagatatagtggtagtaatgaatctgggggatataataatttctattggccagacaatgagacgtttacctcaactataaatcaaaatgtaaatgtaacagtacaagataaatataaagtgagttatgatgagaaaataaacgctattaaagatacattaaatgaaacgattaacaatagtgaagatgttaatcatctatatattaattttacaagcttgtcttctggtggtacagcatggaatagtccatattattatgcgtcctacataaatcctgaaattgcaaattatatgaagcaaaagaatcctacgagagtgggctggataatacaagattatataaatgaaaaatggtcaccattactttatcaagaagttataagagcgaataagtcacttgtaaaatag

SEQ ID NO: 41 - protein, 328 aa, Unknown
  protein obtained from an environmental sample
MNNKKFILKLFICSMVLSAFVFAFNDKKTVAASSINVLENWSRWMKPINDDIPLARISIPGTHDSGTFKLQNPIKQVWGMTQEYDFRYQMDHGARIFDIRGRLTDDNTIVLHHGPLYLYVTLHEFINEAKQFLKDNPSETIIMSLKKEYEDMKGAESSFSSTFEKNYFRDPIFLKTEGNIKLGDARGKIVLLKRYSGSNESGGYNNFYWPDNETFTSTINQNVNVTVQDKYKVSYDEKINAIKDTLNETINNSEDVNHLYINFTSLSSGGTAWNSPYYYASYINPEIANYMKQKNPTRVGWIIQDYINEKWSPLLYQEVIRANKSLVK

SEQ ID NO: 42 - DNA, 1020 bp, Amycolatopsis azurea
atgaaactcgtccccgcggtatccctcgtcggcgtgatcttggcggcttcgctgtccggaacggtcgcgaacgccgacgccgccaaggtctcccacgtgacaacggtcggcgtccacaacacctacgagaccggtgcctacgactacctggcgcgctcgctggacgccggtacctcgctgatcgaactcgacgtctggccgaacatcatcactcgcgagtggaaagtgagtcactcgaacccgttggggaacaacaacaactgcgtcgcggcgagtacgccgtcgcagttgtactccggtgggcggaacaagaacctcgaacactgcctcgatgacatccgcgtctggctgggcgcgcatccggacagcaaaccggtcacgctcaaactggagatgaagaccgggttcgccgacaaccgcggcctcggcccggacgaactcgacgcgtccatccgggcacatctgggcagtacggtgttccggccggcggacctgctcggcgggtacgcgacgctcgacgacgcggccaaagcggacaactggccgtccctggacgcgctgcggggcaaggtgatcatcgagatcatccccggcacggtcgaagagggaaatccgacggacacgctcaagaccgacgtcgagtacgggcgatatctgcggtcgctcaaggacgcgggccgcgtcggcgaggtgcagatcttcccgacggtgcacggcgcggcacccggcgacccgcgcacgaagtacgccgacgccgggctgcggccgtggttcgtggtcttcgacggcgacgccaccgcgttcctgacgcagaccgggcccggctggtacgacgacaaccactactacgtggtgatgaccgacgcgcacaacgtcgcgccggccatcgactcccgtgcccccacggtggaccaggcgagcgcgcgagcggccctgctggcgaagaaccacgcttcggtgctgacctcggattggacggggctgaccaccgtgctgccgcaggtacttccgcgcggctag

SEQ ID NO: 43 - protein, 339 aa, Amycolatopsis azurea
  SIGNAL (1)..(27)
MKLVPAVSLVGVILAASLSGTVANADAAKVSHVTTVGVHNTYETGAYDYLARSLDAGTSLIELDVWPNIITREWKVSHSNPLGNNNNCVAASTPSQLYSGGRNKNLEHCLDDIRVWLGAHPDSKPVTLKLEMKTGFADNRGLGPDELDASIRAHLGSTVFRPADLLGGYATLDDAAKADNWPSLDALRGKVIIEIIPGTVEEGNPTDTLKTDVEYGRYLRSLKDAGRVGEVQIFPTVHGAAPGDPRTKYADAGLRPWFVVFDGDATAFLTQTGPGWYDDNHYYVVMTDAHNVAPAIDSRAPTVDQASARAALLAKNHASVLTSDWTGLTTVLPQVLPRG

SEQ ID NO: 44 - DNA, 771 bp, Artificial sequence
  Synthetic gene
gaagatgacgcacaaccgcctattacagcaaaatggtcagcggaagatccgcatcatgaagatacaaatacacatctgtggattgttcgccatgccatggagattatggcgaataacaaagatgttgtcaaacctggcgaagtcgaacaactgaaacaatggcaatcagatctggaacagggcatttatgatgcagatcatgcaaacccgtattatgataatgcaacatttgcgagccatttttatgatccggatacaggcaaatcatatattccgctggcagcacatgcaaaaacaacaagcgttaaatactttaaaagagcaggcgaagcgtatcagaaaggcgatcataaacaagcgttttacaatctgggcctggcgctgcattatattggcgatctgaatcaaccgatgcatgcagcgaattttacaaatctgtcatatccgcaaggctttcatagcaaatatgaaaactatgtcgatagctttaaagaagattacgcggtcaaagatggcgaaggctattggcattggaaaggcacaaatccggaagattggctgcatggcacagcagttgcagcaaaaaaagattatccggatatcgtcaacgatacaacgaaagcatggtttgttaaagcagcagtctcaaatagctatgcagcaaaatggcgtgcagcagttgttccggcaacaggcaaaagactgacagaagcacaaagaattctggcaggctatatgcaactgtggtttgatacatatgtcaacaaataa

SEQ ID NO: 45 - protein, 280 aa, Bacillus macauensis
  SIGNAL (1)..(24)
  PROPEP (25)..(35)
MKKTFVAVATAALLVTGFQGNASAEDDAQPPITAKWSAEDPHHEDTNTHLWIVRHAMEIMANNKDVVKPGEVEQLKQWQSDLEQGIYDADHANPYYDNATFASHFYDPDTGKSYIPLAAHAKTTSVKYFKRAGEAYQKGDHKQAFYNLGLALHYIGDLNQPMHAANFTNLSYPQGFHSKYENYVDSFKEDYAVKDGEGYWHWKGTNPEDWLHGTAVAAKKDYPDIVNDTTKAWFVKAAVSNSYAAKWRAAVVPATGKRLTEAQRILAGYMQLWFDTYVNK

SEQ ID NO: 46 - protein, 277 aa, Talaromyces leycettanus
APAPVLRRDVSSSVLSELDLFAQYSAAAYCSSNIGSPGTKLTCSVGNCPRVEAADTETLIEFNESSSFGDVTGYIAVDRTNSLLVLAFRGSSTVSNWEADLDFPLTDASSLCSGCEIHSGFWAAWQTVQASITSTLESAIASYPGYTLVFTGHSYGAALAAIAATTLRNAGYTIQLYDYGQPRLGNLALAQYITAQTQGANYRVTHTDDIVPKLPPELFGYHHFSPEYWITSGDNVTVTTSDVQVVTGIDSTAGNDGTLLDSTSAHDWYIVYIDGCD

SEQ ID NO: 47 - protein, 277 aa, Talaromyces leycettanus; identical to SEQ ID NO: 46

(19) United States
(12) Patent Application Publication
(10) Pub. No.: US 2017/0058235 A1
(43) Pub. Date: Mar. 2, 2017

(54) Compositions Comprising Polypeptides Having Phospholipase C Activity and Use Thereof

(71) Applicant: Novozymes A/S, Bagsvaerd (DK)

(72) Inventors: Marianne Linde Damstrup, Gentofte (DK); Jesper Brask, Vaerlose (DK); Hanna Maria Lilbaek, Copenhagen (DK)

(73) Assignee: Novozymes A/S, Bagsvaerd (DK)

(21) Appl. No.: 15/308,495

(22) PCT Filed: May 15, 2015
(86) PCT No.: PCT/EP2015/060819

§ 371 Date: Nov. 2, 2016

(30) Foreign Application Priority Data
May 15, 2014 (EP) 14168437.3

(51) Int. Cl. C11B 3/00; C12N 9/88; C12N 9/16

(52) U.S. Cl. CPC C11B 3/003; C12N 9/16; C12Y 301/04003; C12Y 301/04011; C12N 9/88; C12Y 406/01013

(57) ABSTRACT

The present invention relates to compositions comprising polypeptides having phospholipase C activity and to the use of such compositions for hydrolysing a phospholipid or lysophospholipid, The invention also relates to processes for hydrolysing a phospholipid or lysophospholipid, comprising contacting said phospholipid or lysophospholipid with a composition or a pluralities of polypeptides having Phospholipase C activity according to the invention.

### REFERENCE TO A SEQUENCE LISTING

This application contains a Sequence Listing in computer readable form, which is incorporated herein by reference.

### BACKGROUND OF THE INVENTION

Field of the Invention

The present invention relates to compositions comprising a plurality of polypeptides having phospholipase C activity, wherein at least one of said polypeptides having phospholipase C activity is a polypeptide derived from a fungus, and at least one of said polypeptides having phospholipase C activity is a polypeptide derived from a bacterium.

The invention also relates to such compositions, for use in processes such as for degumming of an aqueous carbohydrate solution or slurry, and for degumming of oil.

The invention further relates to a process for hydrolysing a phospholipid or lysophospholipid, comprising contacting said phospholipid or lysophospholipid with a composition as defined above.

Description of the Related Art

Several types of phospholipases are known which differ in their specificity according to the position of the bond attacked in the phospholipid molecule. Phospholipase A1 (PLA1) removes the 1-position fatty acid to produce free fatty acid and 1-lyso-2-acylphospholipid. Phospholipase A2 (PLA2) removes the 2-position fatty acid to produce free fatty acid and 1-acyl-2-lysophospholipid. The term phospholipase B (PLB) is used for phospholipases having both A1 and A2 activity. Phospholipase C (PLC) removes the phosphate moiety to produce 1,2 diacylglycerol and phosphate ester. Phospholipase D (PLD) produces 1,2-diacylglycero-phosphate and base group (See FIG. 1).

Before consumption vegetable oils are degummed to provide refined storage stable vegetable oils of neutral taste and light color. The degumming process comprises removing the phospholipid components (the gum) from the triglyceride rich oil fraction. The most commonly used processes in the industry are water degumming, chemical/caustic refining and physical refining including acid assisted degumming and/or enzyme assisted degumming. Due to the emulsifying properties of the phospholipid components, the degumming procedure has resulted in a loss of oil i.e. of triglycerides.

Enzymatic degumming reduces the oils loss due to an efficient hydrolysis of the phospholipids which decrease the gum formation and reduce the oil entrained in the gum phase. For a review on enzymatic degumming see Dijkstra 2010 Eur. J. Lipid Sci. Technol. 112, 1178. The use of Phospholipase A and/or phospholipase C in degumming is for example described in Clausen 2001 Eur J Lipid Sci Techno 103 333-340, WO 2003/089620 and WO 2008/094847. Phospholipase A solutions generate lysophospholipid and free fatty acids resulting in oil loss. Phospholipase C on the other hand has the advantage that it produces diglyceride (FIG. 2) which will remain in the oil and therefore will reduce losses. There are four major phospholipids in vegetable oil phosphatidylcholine (PC), phosphatidylethanolamine (PE), phosphatidic acid (PA) and phosphatidyl inositol (PI). Phospholipase C enzymes have different specificity towards these phospholipids. The only known commercially available phospholipase C is (f) PURIFINE® PLC of Verenium/DSM (Dijkstra, 101st AOCS Annual Meeting 10. May 2010) which has specificity towards PC and PE. WO07/059927 describes a thermostable Bacillus PLC for degumming. WO 2012/062817 describes a fungal PLC with specificity towards all four phospholipids. A PI-specific phospholipase C has been described in WO 2011/046815. Despite the availability of such phospholipases, there is a need to improve the efficacy of the enzymatic hydrolysis of phospholipids by decreasing the reaction time of the conversion into diacylglycerol and phosphate ester, while providing broad substrate specificity.

### SUMMARY OF THE INVENTION

The present invention relates to a composition comprising a plurality of polypeptides having phospholipase C activity, wherein
at least one of said polypeptides having phospholipase C activity is a polypeptide derived from a fungus; and
at least one of said polypeptides having phospholipase C activity is a polypeptide derived from a bacterium.

The invention further provides such a composition, for use in
i) degumming of an aqueous carbohydrate solution or slurry, in degumming of oil, e.g. vegetable oil, or an edible vegetable oil, or in a process comprising hydrolysis of phospholipids in the gum fraction from water degumming to release entrapped triglyceride oil,
ii) a process comprising hydrolysis of phospholipids to obtain improved phospholipid emulsifiers, in particular wherein said phospholipid is lecithin,
iii) a process for improving the filterability of an aqueous solution or slurry of carbohydrate origin which contains phospholipid,
iv) a process for the extraction of oil,
v) a process for the production of an animal feed product,
vi) a process for the production of a biofuel, e.g. a biodiesel,
vii) in a process for the production of a detergent product, and/or
viii) in a process for making a baked product, comprising adding the phospholipase to a dough, and baking the dough to make the baked product.

Finally, the invention provides a process for hydrolysing a phospholipid or lysophospholipid, comprising contacting said phospholipid or lysophospholipid with a plurality of polypeptides having phospholipase C activity and/or a composition according to the invention.

### BRIEF DESCRIPTION OF THE FIGURES

FIG. 1 illustrates where different phospholipases cleave a phospholipid as well as the four major functional groups on phospholipids.

FIG. 2 illustrates the reaction of a phospholipid with a phospholipase C to form diglyceride and a phosphate ester or phosphoric acid.

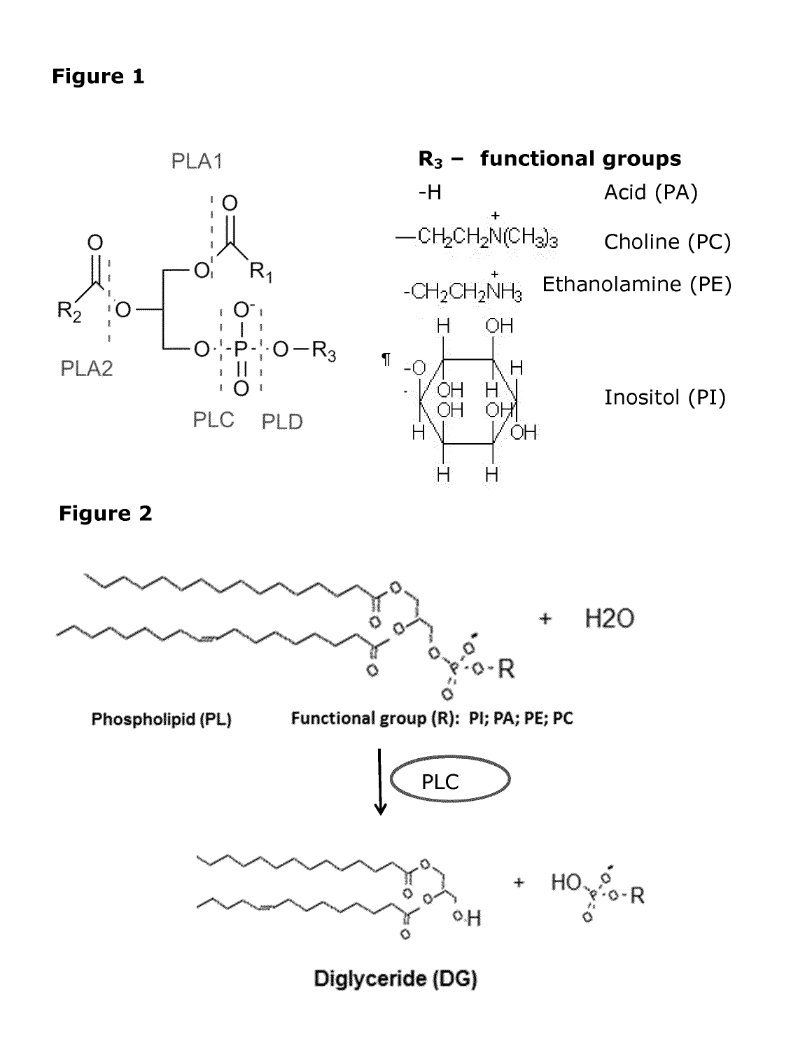

### DEFINITIONS

Phospholipase C activity: The term “phospholipase C activity” or “PLC activity” relates to an enzymatic activity that removes the phosphate ester moiety from a phospholipid to produce a 1,2 diacylglycerol (see FIG. 2). Most PLC enzymes belong to the family of hydrolases and phosphodiesterases and are generally classified as EC 3.1.4.3. Some PLC enzymes are classified in other EC classes, for example PI-specific PLC's. Phospholipase C activity may be determined according to the procedure described in Example 1 or by one of the assays described in the “Assay for phospholipase activity” section.

Phospholipase A activity: In the context of the present invention the term “phospholipase A activity” comprises enzymes having phospholipase A1 and/or phospholipase A2 activity (A1 or A2, EC 3.1.1.32 or EC 3.1.1.4), i.e., hydrolytic activity towards one or both carboxylic ester bonds in phospholipids such as lecithin. A phospholipases having both A1 and A2 activity is also referred to as a phospholipase B.

Phospholipase A activity may be determined by one of the assays described in the “Assay for phospholipase activity” section. For purposes of the present invention, phospholipase A activity may also be determined using a LEU assay, in which the phospholipase A activity is determined from the ability to hydrolyze lecithin at pH 8.0, 40° C. The hydrolysis reaction can be followed by titration with NaOH for a reaction time of 2 minutes. The phospholipase from Fusarium oxysporum (LIPOPAN F) disclosed in WO 1998/26057 has an activity of 1540 LEU/mg enzyme protein and may be used as a standard.

In one aspect, the polypeptides of the present invention have at least 20%, e.g., at least 40%, at least 50%, at least 60%, at least 70%, at least 80%, at least 90%, at least 95%, or at least 100% of the phospholipase A activity of the mature polypeptide of SEQ ID NO: 46 and/or of the polypeptide of SEQ ID NO: 47.

In addition to having phospholipase A activity, the polypeptides of the present invention may also have lipase activity.

Assays for phospholipase activity: Routine protocols for determining the activity phospholipase A, C, are well known in the art.

Exemplary activity assays include turbidity assays, methylumbelliferyl phosphocholine (fluorescent) assays, Amplex red (fluorescent) phospholipase assays, thin layer chromatography assays (TLC), cytolytic assays and p-nitrophenylphosphorylcholineassays. Using these assays polypeptides, peptides or antibodies can be quickly screened for a phospholipase activity.

Plate assays with a substrate containing agar can be used to determine phospholipase activity. Useful substrates are lecithin or specific phospholipids. The assay can be conducted as follows. Plates are casted by mixing of 5 ml 2% Agarose (Litex HSA 1000) prepared by mixing and cooking in buffers (100 mM HEPES and 100 mM Citrate with pH adjusted from pH 3.0 to pH 7.0) for 5 minutes followed by cooling to approximately 60° C. and 5 ml substrate (L-alfa Phosohatidylcholine, 95% from Soy (Avanti 441601) or L-α-phosphatidylinositol from Soy (Avanti 840044P) for PI-specificity or L-α-phosphatidylethanolamine from Soy (Avanti 840024P) or lecithin) dispersed in water (MilliQ) at 60° C. for 1 minute with Ultra Turrax for PC-specificity) gently mixed into petri dishes with diameter of 7 cm and cooled to room temperature before holes with a diameter of approximately 3 mm were punched by vacuum. Ten microliters of purified enzyme diluted to 0.4 mg/ml is added into each well before plates were sealed by parafilm and placed in an incubator at 55° C. for 48 hours. Plates were taken out for photography at regular intervals.

Turbidity assays to determine phospholipase activity are described, e.g., in Kauffmann (2001) “Conversion of Bacillus thermocatenulatus lipase into an efficient phospholipase with increased activity towards long-chain fatty acyl substrates by directed evolution and rational design,” Protein Engineering 14:919-928; Ibrahim (1995) “Evidence implicating phospholipase as a virulence factor of Candida albicans,” Infect. Immun. 63:1993-1998.

Methylumbelliferyl (fluorescent) phosphocholine assays to determine phospholipase activity are described, e.g., in Goode (1997) “Evidence for cell surface internal phospholipase activity in ascidian eggs,” Develop. Growth Differ. 39:655-660; Diaz (1999) “Direct fluorescence-based lipase activity assay,” BioTechniques 27:696-700.

Amplex Red (fluorescent) Phospholipase Assays to determine phospholipase activity are available as kits, e.g., the detection of phosphatidylcholine-specific phospholipase using an Amplex Red phosphatidylcholine-specific phospholipase assay kit from Molecular Probes Inc. (Eugene, Oreg.), according to manufacturer's instructions.

Fluorescence is measured in a fluorescence microplate reader using excitation at 560±10 nm and fluorescence detection at 590±10 nm. The assay is sensitive at very low enzyme concentrations.

Thin layer chromatography assays (TLC) to determine phospholipase activity are described, e.g., in Reynolds (1991) Methods in Enzymol. 197:3-13; Taguchi (1975) “Phospholipase from Clostridium novyi type A.I,” Biochim. Biophys. Acta 409:75-85. Thin layer chromatography (TLC) is a widely used technique for detection of phospholipase activity. Various modifications of this method have been used to extract the phospholipids from the aqueous assay mixtures. In some PLC assays the hydrolysis is stopped by addition of chloroform/methanol (2:1) to the reaction mixture. The unreacted starting material and the diacylglycerol are extracted into the organic phase and may be fractionated by TLC, while the head group product remains in the aqueous phase. For more precise measurement of the phospholipid digestion, radio labeled substrates can be used (see, e.g., Reynolds (1991) Methods in Enzymol. 197:3-13). The ratios of products and reactants can be used to calculate the actual number of moles of substrate hydrolyzed per unit time. If all the components are extracted equally, any losses in the extraction will affect all components equally. Separation of phospholipid digestion products can be achieved by silica gel TLC with chloroform/methanol/water (65:25:4) used as a solvent system (see, e.g., Taguchi (1975) Biochim. Biophys. Acta 409:75-85).

p-Nitrophenylphosphorylcholine assays to determine phospholipase activity are described, e.g., in Korbsrisate (1999) J. Clin. Microbiol. 37:3742-3745; Berka (1981) Infect. Immun. 34:1071-1074. This assay is based on enzymatic hydrolysis of the substrate analog p-nitrophenylphosphorylcholine to liberate a yellow chromogenic compound p-nitrophenol, detectable at 405 nm. This substrate is convenient for high throughput screening. Similar assays using substrates towards the other phospholipid groups can also be applied e.g. using p-nitrophenylphosphorylinositol or p-nitrophenylphosphorylethanolamine.

A cytolytic assay can detect phospholipases with cytolytic activity based on lysis of erythrocytes. Toxic phospholipases can interact with eukaryotic cell membranes and hydrolyze phosphatidylcholine and sphingomyelin, leading to cell lysis. See, e.g., Titball (1993) Microbiol. Rev. 57:347-366.

Further assays like ³¹P-NMR and Liquid Chromatography coupled to triple quadrupole mass spectrometer (LC/MS/MS) are described in the example section of this application.

PC and PE-specific phospholipase C: The terms “PC and PE-specific phospholipase C” and “PC, PE-specific phospholipase C” and “polypeptide having activity towards phosphatidylcholine (PC) and phosphatidylethanolamine (PE)” are used interchangeably. They relate to a polypeptide having activity towards phosphatidylcholine (PC), phosphatidylethanolamine (PE). In addition to the PC and PE specificity it may also have some activity towards phosphatidic acid (PA) and phosphatidyl inositol (PI). Preferably a PC and PE specific phospholipase C removes at least 30% PC and at least 30% PE from an oil or fat with at least 100 ppm PC and 100 ppm PE when using the P-NMR assay of Example 1 at the optimal pH of the enzyme and an enzyme dosage of 10 mg/kg. More preferably it removes 40%, 50%, 60%, 70% or 80%, even more preferred it removes 90% and most preferred it removes between 90% and 100% of the PC in the oil or fat and 40%, 50%, 60%, 70% or 80%, even more preferred it removes 90% and most preferred it removes between 90% and 100% of the PE in the oil or fat.

PI-Specific Phospholipase C: The terms “PI-specific phospholipase C”,

“Phosphatidylinositol phospholipase C” and and “polypeptide having activity towards phosphatidylinositol (PI)” are used interchangeably. They relate to a polypeptide having activity towards phosphatidyl inositol (PI), meaning that its activity towards phosphatidylcholine (PC), phosphatidylethanolamine (PE), phosphatidic acid (PA) is low compared to the PI activity. PI-specific phospholipase C enzymes can either belong to the family of hydrolases and phosphodiesterases classified as EC 3.1.4.11 or to the family of lyases classified as EC 4.6.1.13. PI-specific phospholipase C activity may be determined according to the procedure described in Example 5. Preferably a PI-specific phospholipase C removes at least 30% PI from an oil or fat with at least 50 ppm PI when using the P-NMR assay of Example 1 at the optimal pH of the enzyme and an enzyme dosage of 10 mg/kg. More preferably it removes 40%, 50%, 60%, 70% or 80%, even more preferred it removes 90% and most preferred it removes between 90% and 100% of the PI in the oil or fat.

Preferably a PI-specific Phospholipase C removes at least 20% more PI when compared to the amount of PC, PE or PA it can remove, more preferred at least 30%, 40%, even more preferred at least 50% and most preferred at least 60% more PI when compared to the amount of PC, PE or PA it can remove.

PC-, PE-, PA- and PI-Specific Phospholipase C: The terms “PC-, PE-, PA,- and PI-specific phospholipase C”, and “polypeptide having activity towards phosphatidylcholine (PC), phosphatidylethanoamine (PE), phosphatidic acid (PA) and phosphatidylinositol (PI)” are used interchangeably. They relate to a polypeptide having activity towards phosphatidylcholine (PC), phosphatidylethanoamine (PE), phosphatidic acid (PA), and phosphatidyl inositol (PI). Preferably a PC-, PE-, PA,- and PI-specific phospholipase C removes at least 30% of each of the four phospholipid species from an oil or fat with at least 100 ppm PC, 75 ppm PE, 5 ppm PA and 50 ppm PI when using the P-NMR assay of Example 1 at the optimal pH of the enzyme and an enzyme dosage of 10 mg/kg. More preferably it removes 40%, 50%, 60%, 70% or 80%, even more preferred it removes 90% and most preferred it removes between 90% and 100% of the PC in the oil or fat and 40%, 50%, 60%, 70% or 80%, even more preferred it removes 90% and most preferred it removes between 90% and 100% of the PE in the oil or fat.

Bacterial Phospholipase C/Phospholipase C derived from a bacterium: For purposes of the present invention, the term “derived from” as used herein in connection with a given source shall mean that the polypeptide is encoded by a polynucleotide, which in its native form is present in that source, and that the polypeptide is produced by the source or by a strain (“host cell”) in which the polynucleotide from the source has been inserted. The term is also used in connection with polypeptides which are encoded by a modified form of a polynucleotide from the source, wherein the polynucleotides have been modified, such as by substitution, deletion, insertion or addition of one or more nucleic acid residues. Hence, the terms “bacterial Phospholipase C”, “Phospholipase C derived from a bacterium” and “polypeptide having phospholipase C activity and being derived from a bacterium” are used interchangeably to refer to a polypeptide having Phospholipase C activity, which is encoded by a polynucleotide, which in its native form is present in bacterium, or is encoded by a modified form of that polynucleotide. In one aspect, the polypeptide obtained from a given source or host cell is secreted extracellularly.

Also comprised by this definition is a polypeptide, which has phospholipase C activity, and is selected from the group consisting of:

(a) a polypeptide having at least 60%, such as at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of a polypeptide having phospholipase C activity, and being encoded by a polynucleotide, which in its native form is present in a bacterium;

(b) a fragment of the polypeptide defined in (a);

including such polypeptides having a length of 280-320 amino acid residues, such as a length of 280-310 amino acid residues, 280-305 amino acid residues, 280-300 amino acid residues, 280-298 amino acid residues 280-297 amino acid residues, 280-296 amino acid residues, 285-320 amino acid residues, 285-315 amino acid residues, 285-310 amino acid residues, 285-305 amino acid residues, 285-300 amino acid residues, 285-298 amino acid residues, 285-297 amino acid residues, 285-296 amino acid residues, 290-320 amino acid residues, 290-315 amino acid residues, 290-310 amino acid residues, 290-305 amino acid residues, 290-300 amino acid residues, 290-298 amino acid residues, 290-297 amino acid residues, 290-296 amino acid residues, 295-320 amino acid residues, 295-315 amino acid residues, 295-310 amino acid residues, 295-305 amino acid residues, 295-300 amino acid residues, 295-298 amino acid residues, 255-297 amino acid residues, or a length of 295-296 amino acid residues.

Fungal Phospholipase C/Phospholipase C derived from a fungus: In accordance with the above, the terms “fungal Phospholipase C” “Phospholipase C derived from a fungus”, and “polypeptide having phospholipase C activity and being derived from a fungus” are used interchangeably to refer to a polypeptide having Phospholipase C activity, which is encoded by a polynucleotide, which in its native form is present in a fungus, or is encoded by a modified form of that polynucleotide.

Also comprised by this definition is a polypeptide, which has phospholipase C activity, and is selected from the group consisting of:

(a) a polypeptide having at least 60%, such as at at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of a polypeptide having phospholipase C activity, and being encoded by a polynucleotide, which in its native form is present in a fungus;

(b) a fragment of the polypeptide defined in (a);

including such polypeptides having a length of 540-640 amino acid residues, such as a length of, 550-640 amino acid residues, 560-640 amino acid residues, 570-640 amino acid residues, 540-630 amino acid residues, such as a length of, 550-630 amino acid residues, 560-630 amino acid residues, 570-630 amino acid residues, 570-620 amino acid residues, 570-610 amino acid residues, 570-600 amino acid residues 570-597 amino acid residues, 570-596 amino acid residues, 580-640 amino acid residues, 540-630 amino acid residues, such as a length of, 540-620 amino acid residues, 540-610 amino acid residues, 540-600 amino acid residues, 540-590 amino acid residues, 540-580 amino acid residues, 540-570 amino acid residues, 540-560 amino acid residues, 540-560 amino acid residues, 550-630 amino acid residues, such as a length of, 550-620 amino acid residues, 550-610 amino acid residues, 550-600 amino acid residues, 550-590 amino acid residues, 550-580 amino acid residues, 550-570 amino acid residues, 550-560 amino acid residues, 550-560 amino acid residues, 560-630 amino acid residues, such as a length of, 560-620 amino acid residues, 560-610 amino acid residues, 560-600 amino acid residues, 560-590 amino acid residues, 560-580 amino acid residues, 560-570 amino acid residues, 560-560 amino acid residues, 560-560 amino acid residues, 570-630 amino acid residues, such as a length of, 570-620 amino acid residues, 570-610 amino acid residues, 570-600 amino acid residues, 570-590 amino acid residues, 570-580 amino acid residues, 570-570 amino acid residues, 570-560 amino acid residues, 570-560 amino acid residues, 580-630 amino acid residues, 580-620 amino acid residues, 580-610 amino acid residues, 580-600 amino acid residues, 580-598 amino acid residues, 580-597 amino acid residues, 580-596 amino acid residues, 590-640 amino acid residues, 590-630 amino acid residues, 590-620 amino acid residues, 590-610 amino acid residues, 590-600 amino acid residues, 590-698 amino acid residues, 590-597 amino acid residues, 590-596 amino acid residues, 595-640 amino acid residues, 595-630 amino acid residues, 595-620 amino acid residues, 595-610 amino acid residues, 595-600 amino acid residues, 595-598 amino acid residues, 595-597 amino acid residues, or a length of 595-596 amino acid residues.

Reaction time: For the purpose of the present invention the “reaction time” of a phospholipase C is the time required to convert 50% of the content in crude soybean oil of the phospholipid towards which the phospholipase C has activity, when determined in a degumming assay comprising: Adding Ortho Phosphoric acid (85% solution) to a sample of said oil in amounts equal to 0.05% (100% pure Ortho Phosphoric acid) based on oil amount and mixing the sample in ultrasonic bath for 5 minutes, incubating the sample in rotator for 15 minutes followed by base neutralization with 4 M NaOH applied in equivalents to pure Ortho Phosphoric acid in ultrasonic bath for 5 minutes, adding said phospholipase C in amounts corresponding to 10 mg enzyme protein/kg oil in low aqueous system (3% water total based on oil amount), subjecting the sample to ultrasonic treatment and incubation at a temperature 50-60° C. with stirring, and subjecting the oil to centrifugation at 700 g at 85° C. for 15 minutes. An example of the degumming assay and data obtained thereby is provided in Example 1

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Fragment: The term “fragment” means a polypeptide or a catalytic or phospholipid binding domain having one or more (e.g., several) amino acids absent from the amino and/or carboxyl terminus of a mature polypeptide or domain; wherein the fragment has phospholipase C activity.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide may be predicted, such as by using the software SignalP (Nielsen et al., 1997, Protein Engineering 10: 1-6)]

Mature polypeptide coding sequence: The term “mature polypeptide coding sequence” means a polynucleotide that encodes a mature polypeptide having phospholipase C activity. A mature polypeptide sequence may be predicted using the software SignalP (Nielsen et al., 1997, supra).

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

Subsequence: When used in connection with nucleic acid sequences, the term “subsequence” means a polynucleotide having one or more (e.g., several) nucleotides absent from the 5′ and/or 3′ end of a mature polypeptide coding sequence; wherein the subsequence encodes a fragment having phospholipase C activity.

Variant: The term “variant” means a polypeptide having phospholipase C activity comprising one or more alterations, i.e., one or more substitutions, insertions, and/or deletions, at one or more (e.g., several) positions. A substitution means replacement of the amino acid residue occupying a position with a different amino acid residue; a deletion means removal of the amino acid residue occupying a position; and an insertion means adding one or more amino acid residues adjacent to and immediately following the amino acid residue occupying a position.

Crude oil: The term “crude oil” refers to (also called a non-degummed oil) a pressed or extracted oil or a mixture thereof from, e.g. vegetable sources, including but not limited to acai oil, almond oil, babassu oil, blackcurrent seed oil, borage seed oil, canola oil, cashew oil, castor oil, coconut oil, coriander oil, corn oil, cottonseed oil, crambe oil, flax seed oil, grape seed oil, hazelnut oil, hempseed oil, jatropha oil, jojoba oil, linseed oil, macadamia nut oil, mango kernel oil, meadowfoam oil, mustard oil, neat's foot oil, olive oil, palm oil, palm kernel oil, palm olein, peanut oil, pecan oil, pine nut oil, pistachio oil, poppy seed oil, rapeseed oil, rice bran oil, safflower oil, sasanqua oil, sesame oil, shea butter, soybean oil, sunflower seed oil, tall oil, tsubaki oil walnut oil, varieties of “natural” oils having altered fatty acid compositions via Genetically Modified Organisms (GMO) or traditional “breading” such as high oleic, low linolenic, or low saturated oils (high oleic canola oil, low linolenic soybean oil or high stearic sunflower oils).

Degummed oil: The term “degummed oil” refers to an oil obtained after removal of nonhydratable phospholipids, hydratable phospholipids, and lecithins (known collectively as “gums”) from the oil to produce a degummed oil or fat product that can be used for food production and/or non-food applications, e.g. biodiesel. In certain embodiments, the degummed oil has the phospholipids content of less than 200 ppm phosphorous, such as less than 150 ppm phosphorous, less than 100 ppm phosphorous, less than (or less than about) 50 ppm phosphorous, less than (or less than about) 40 ppm phosphorous, less than (or less than about) 30 ppm phosphorous, less than (or less than about) 20 ppm phosphorous, less than (or less than about) 15 ppm phosphorous, less than (or less than about) 10 ppm phosphorous, less than (or less than about) 7 ppm phosphorous, less than (or less than about) 5 ppm phosphorous, less than (or less than about) 3 ppm phosphorous or less than (or less than about) 1 ppm phosphorous

### DETAILED DESCRIPTION OF THE INVENTION

The present inventors have observed that very efficient hydrolysis of phospholipids in processes such as degumming of oils may be obtained by combining fungal phospholipase C, characterized by broad substrate specificity with bacterial phospholipase C characterized by faster, but more specific activity of bacterial phospholipase.

### Enzyme Compositions

Hence, in a first aspect the present invention provides a composition comprising a plurality of polypeptides having phospholipase C activity, wherein
at least one of said polypeptides having phospholipase C activity is a polypeptide derived from a fungus; and
at least one of said polypeptides having phospholipase C activity is a polypeptide derived from a bacterium.

In particular embodiments of the invention, the composition comprises 2 or more polypeptides, such as 2, 3, 4, 5, or 6 polypeptides having phospholipase C activity, each of which is derived from a fungus.

According to other embodiments of the invention the composition comprises 2 or more polypeptides, such as 1, 2, 3, 4, 5, or 6 polypeptides having phospholipase C activity, each of which is derived from a bacterium.

In particular, the invention provides compositions in which one or more of said polypeptides derived from a fungus has/have broad substrate acceptance, such as activity towards two, three or four phospholipids selected from the group consisting of phosphatidylcholine (PC), phosphatidylethanoamine (PE), phosphatidic acid (PA), and phosphatidyl inositol (PI). According to some embodiments, one or more of said polypeptides derived from a fungus has/have activity towards phosphatidylcholine (PC), phosphatidylethanoamine (PE), phosphatidic acid (PA), and phosphatidyl inositol (PI). In general, the polypeptides having phospholipase C activity which are derived from bacteria have relatively fast activity and short reaction time. Hence, according to some embodiments at least one of said polypeptides derived from a bacterium, such as at least 2, at least 3, at least 4, at least 5 or at least 6 of said polypeptides derived from a bacterium has/have a reaction time of 1-5 hours, such as 1-4 hours, 1-3 hours, 1.5-3.5 hours, 1.5-2.5 hours, 2-4 hours, such as 1 hour, 2 hours, 3 hours, 4 hours or 5 hours.

According to some embodiments, the invention provides compositions in which one or more of said polypeptides derived from a bacterium has/have a narrower substrate acceptance, such as activity towards one, two or three phospholipids selected from the group consisting of phosphatidylcholine (PC), phosphatidylethanoamine (PE), and phosphatidyl inositol (PI). In particular embodiments, the invention provides compositions, wherein one or more of said polypeptides derived from a bacterium has activity towards phosphatidyl inositol (PI) and/or is a PI-specific phospholipase C. According to additional embodiments, one or more of said polypeptides derived from a bacterium has/have activity towards phosphatidylcholine (PC) and phosphatidylethanoamine (PE) and/or is a PC- and PE-specific phospholipase C.

In general, the polypeptides having phospholipase C activity which are derived from fungi need long reaction time to convert all phospholipid species (PI; PA; PE; PC). Accordingly, in some embodiments, at least one of said polypeptides derived from a fungus, such as at least 2, at least 3, at least 4, at least 5 or at least 6 of said polypeptides derived from a fungus has/have a has a reaction time of 3-36 hours, such as 3-30 hours, or such as 4-24 hours.

The fungus (kingdom Fungi) could be a dicaryotic fungus, more specifically from the fungal phylum Ascomycota, more specifically from a filamentous ascomycete from the subphylum Pezizomycotina, more specifically from the fungal classes Sordariomycetes or Eurotiomycetes; more specifically from the fungal orders Sordariales, Hypocreales and Eurotiales, more specifically from the fungal families Nectriaceae and Aspergillaceae; more specifically from the fungal genera Kionochaeta, Nectria, and Penicillium.

While several fungal sources are available, some embodiments of the Invention pertain to compositions in which one or more of said polypeptides derived from a fungus, such as 2, 3, 4, 5 or 6 of said polypeptides derived from a fungus is/are selected from the group consisting of a polypeptide derived from Kionochaeta sp., a polypeptide derived from Penicillium emersonii and a polypeptide derived from Nectria marianneae. Likewise, the skilled person will be aware of numerous sources of bacterial polypeptides having Phospholipase C activity. The bacterial source (superkingdom Bacteria) could be members of the phyla Firmicutes or Proteobacteria, more specifically from the classes Bacilli or Gammaproteobacteria, more specifically from the bacterial orders Bacillales or Pseudomonades, more specifically from the bacterial families Bacillaceae, Listeriaceae, or Pseudomonadaceae; more specifically from the bacterial genera Bacillus, Listeria, or Pseudomonas. Hence, in specific embodiments of the invention one or more of said polypeptides derived from a bacterium is/are selected from the group consisting of a polypeptide derived from a Bacillus species, a polypeptide derived from a Listeria species, a polypeptide derived from a pseudomonas species. In particular, the said Bacillus species may be selected from the group consisting of Bacillus alkalophilus, Bacillus amyloliquefaciens, Bacillus brevis, Bacillus cereus, Bacillus circulans, Bacillus clausii, Bacillus coagulans, Bacillus firmus, Bacillus lautus, Bacillus lentus, Bacillus licheniformis, Bacillus megaterium, Bacillus pseudomycoides, Bacillus mycoides, Bacillus pumilus, Bacillus stearothermophilus, Bacillus subtilis and Bacillus thuringiensis.

In specific embodiments, the said Listeria species is Listeria innocua.

In other specific embodiments the said Pseudomonas species is selected from the group consisting of Pseudomonas chlororaphis and Pseudomonas protegens.

Strains of the various species mentioned above are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

In particular embodiments of the invention, the at least one polypeptide having phospholipase C activity and being derived from a fungus has a size/molecular weight which is at least 38 kDa, such as within the range of 40-65 kDa, the molecular weight being calculated from the amino acid sequence of the polypeptide, excluding the amino acid sequence of any signal peptide and/or propeptide.

The at least one polypeptide having phospholipase C activity and being derived from a fungus may further be characterized by having a catalytic site defined by amino acid residues D171, H173, D245, H249, N285, H286, H393, H427 and H429, using the amino acid numbering of SEQ ID NO: 2. Preferably, amino acid residues D171, H173, D245, N285, H393, H427 and H429 are zink-binding residues, whereas H249 and H286 are not.

In the at least one polypeptide having phospholipase C activity and being derived from a fungus the length of the catalytic segment of the amino acid sequence; i.e. the sequence from first (N-terminal) to last (C-terminal) catalytic amino acid residue, preferably is within the range of 250-265 amino acid residues, such as 255-261 amino acid residues or such as 256-260 amino acid residues.

Also, in the at least one polypeptide having phospholipase C activity and being derived from a fungus the length of the amino acid sequence before the first catalytic site; i.e. from the N-terminus of the mature polypeptide to the first (N-terminal) catalytic amino acid residue, is from 100-200 amino acid residues, such as from 100-180 amino acid residues, from 100-170, from 100-160, from 100-150, from 110-180, from 120-180, from 130-180, from 140-180, from 150-180, from 110-170, from 120-170, or from 130-170 amino acid residues.

Further, in the at least one polypeptide having phospholipase C activity and being derived from a fungus the length of the amino acid sequence after the last catalytic site; i.e. from the last (C-terminal) catalytic amino acid residue to the C-terminus of the mature polypeptide, is from 100-200 amino acid residues, such as from 100-190 amino acid residues, from 100-180, from 100-175, from 110-200, from 110-190 from 110-180, from 110-175, from 120-200, from 120-190, from 120-180, from 120-175, from 130-200, from 130-190, from 130-180, from 130-175, from 140-200, from 140-190, from 140-180, from 140-170, from 150-200, from 150-190, from 150-180, or such as from 150-175 amino acid residues.

According to some embodiments, the at least one polypeptide having phospholipase C activity and being derived from a fungus comprises a calcineurin-like phosphoesterase domain as defined by CATH ID 3.60.21.10. Hence, the at least one polypeptide having phospholipase C activity and being derived from a fungus may be selected from polypeptides, which are identifiable (through standard alignment methods/tools such as those provided in the definition of “sequence identity”) as having calcineurin-like phosphoesterase domain as defined by CATH ID 3.60.21.10, starting at position 130-170 in the mature polypeptide, such as at position 130-150 and aligning to the rest of the sequence (allowing for insertions in the domain), In some embodiments of the invention, the said polypeptide(s) having activity towards phosphatidyl inositol (PI)/said PI-specific phospholipase C, and said polypeptides having activity towards phosphatidylcholine (PC) and phosphatidylethanolamine (PE)/said PC- and PE-specific phospholipase C is/are capable of reducing the content of the respective phospholipids present in a crude oil, such as crude soybean oil, by at least 30% (w/w), such as 35% (w/w), 40% (w/w), 45% (w/w), 50% (w/w), 55% (w/w), 60% (w/w), 65% (w/w), 70% (w/w), 75% (w/w), 80% (w/w), 85% (w/w), 90% (w/w), 95% (w/w), 98% (w/w), 99% (w/w) or such as 100% (w/w), when applied in an amount of 10 mg polypeptide/kg oil at the optimal pH, such as a pH within the range of 4.5-8.5, such as in the range of 5.0-8.0 or such as in the range of 6.5-7.5 and incubated for 2-4 hours at a temperature of 50-60° C.

According to some embodiments the polypeptide(s) having activity towards phosphatidylinositol/said PI-specific phospholipase C used according to the invention may be able to reduce the phosphatidylinositol content of crude soy bean oil by 50% or more, 50%, such as by 55%, by 60%, by 65%, by 70%, by 75%, by 80%, by 85%, by 90% or by 95% or more, the reduction in phosphatidylinositol content being determined by ³¹P-NMR after addition of 100 mg enzyme protein(EP)/kg oil and incubation of the oil and enzyme at 50° C. for 2 hours at pH 5.5.

Preferably, the polypeptide(s) having activity towards phosphatidylinositol/the PI-specific phospholipase C according to the invention is/are able to reduce the phosphorous content of crude soy bean oil to 20 mg/kg oil or less as determined by Inductively coupled plasma optical emission spectrometry (ICP-OES) after incubation of 4 mg enzyme protein/kg oil in a low aqueous system comprising 3% water based on oil amount at 50-60° C. for 5 hours.

In further embodiments the polypeptide having activity towards PC and PE/the PC- and PE-specific phospholipase C is able to reduce the phosphatidyl ethanolamine and/or phosphatidyl choline content of crude soy bean oil by 50% or more, 50%, such as by 55%, by 60%, by 65%, by 70%, by 75%, by 80%, by 85%, by 90% or by 95% or more, the reduction in phosphatidyl ethanolamine and/or phosphatidyl choline content being determined by ³¹P-NMR after addition of 100 mg enzyme protein(EP)/kg oil and incubation of the oil and enzyme at 50° C. for 2 hours at pH 5.5.

In still further embodiments, the polypeptide having polypeptide having activity towards PC and PE/the PC- and PE-specific phospholipase C is/are able to reduce the phosphorous content of crude soy bean oil to 20 mg/kg oil or less as determined by Inductively coupled plasma optical emission spectrometry (ICP-OES) after incubation of 4 mg enzyme protein/kg oil in a low aqueous system comprising 3% water based on oil amount at 50-60° C. for 5 hours.

The ability of the polypeptides to reduce phosphorous content may in particular be determined using a crude soy oil which comprises 80-140 ppm phosphorous present as phosphatidic acid (PA), 140-200 ppm phosphorous present as phosphatidyl ethanolamine (PE), 70-110 ppm phosphorous present as phosphatidic acid (PI) and 130-200 ppm phosphorous present as phosphatidyl choline; the phosphorous content being measured by ³¹P-NMR.

Further, said reduction of phosphorous content may be obtained in an oil degumming process comprising the steps of:
i) Optionally treating crude soy bean oil with acid/base by adding an 85% solution of Ortho Phosphoric acid in amounts corresponding to 0.05% (100% pure Ortho Phosphoric acid) based on oil amount, mixing in ultrasonic bath for 5 minutes, followed by incubation in rotator for 15 minutes and base neutralization with 4 M NaOH applied in equivalents (from 0.5 to 0.15) to pure Ortho Phosphoric acid in ultrasonic bath for 5 minutes;
ii) Adding the polypeptide to the oil in amounts of 4 mg enzyme protein/kg oil in a low aqueous system comprising 3% water based on oil amount and subjecting the oil and the polypeptide to ultrasonic treatment for 5 minutes;
iii) Incubating the polypeptide and oil at 50-60° C. for 5 hours with stirring at 20 rpm;
iv) Centrifuging the oil and the polypeptide at 700 g at 85° C. for 15 minutes.

Likewise, in some embodiments the composition according to the invention said polypeptide(s) having activity towards phosphatidylcholine (PC), phosphatidylethanolamine (PE), phosphatidic acid (PA), and phosphatidyl inositol (PI) is/are capable of reducing the content of the respective phospholipids present in a crude oil, such as crude soybean oil, by at least 30% (w/w), such as 35% (w/w), 40% (w/w), 45% (w/w), 50% (w/w), 55% (w/w), 60% (w/w), 65% (w/w), 70% (w/w), 75% (w/w), 80% (w/w), 85% (w/w), 90% (w/w), 95% (w/w), 98% (w/w), 99% (w/w) or such as 100% (w/w), when applied in an amount of 100 mg polypeptide/kg oil at the optimal pH, such as a pH within the range of 4.5-8.5, such as in the range of 5.0-8.0 or such as in the range of 6.5-7.5 and incubated for 2-4 hours at a temperature of 50-60° C.

The reduction of PA, PC, PE and PI content may in particular be determined by ³¹P-NMR after addition of 100 mg enzyme protein(EP)/kg oil and incubation of the oil and enzyme at 50° C. for 2 hours at pH 5.5.

The ability of the polypeptide to reduce phosphorous content and increase dicylglyceride content may in particular be determined using a crude soy oil which comprises 80-140 ppm phosphorous present as phosphatidic acid (PA), 140-200 ppm phosphorous present as phosphatidyl ethanolamine (PE), 70-110 ppm phosphorous present as phosphatidic acid (PI) and 130-200 ppm phosphorous present as phosphatidyl choline; the phosphorous content being measured by ³¹P-NMR and the diacylglycerol content being measured by HPLC-Evaporative Light Scattering Detection (HPLC-ELSD).

In particular, the polypeptide of the present invention is capable of increasing the amount of diacylglyceride by at least 0.1% w/w when applied in amounts of 8.5 mg EP/kg oil to crude soy bean oil and incubated for 3 hours. Preferably, the oil has been acid/base treated by addition of 85% solution of Ortho Phosphoric acid in amounts corresponding to 0.05% (100% pure Ortho Phosphoric acid) based on oil amount, and base neutralization with 4 M NaOH applied in equivalents of 0.5 to pure Ortho Phosphoric acid.

Preferably, the polypeptide of the present invention is capable of increasing the amount of diacylglyceride by at least 0.3% w/w when applied in amounts of 8.5 mg EP/kg to crude soybean oil and incubated with the oil for 3 hours at 50° C., when the crude oil has been acid/base treated with 0.05% Ortho Phosphoric acid and 1 eqv. NaOH.

The reduction of PA, PC, PE and PI content and/or production of diacylglyceride may in particular be obtained in an oil degumming process comprising the steps of:
i) Optionally treating crude soy bean oil with acid/base by adding an 85% solution of Ortho Phosphoric acid in amounts corresponding to 0.05% (100% pure Ortho Phosphoric acid) based on oil amount, mixing in ultrasonic bath for 5 minutes, followed by incubation in rotator for 15 minutes and base neutralization with 4 M NaOH applied in equivalents (from 0.5 to 0.15) to pure Ortho Phosphoric acid in ultrasonic bath for 5 minutes;
ii) Adding the polypeptide to the oil in a low aqueous system comprising 3% water based on oil amount and subjecting the oil and the polypeptide to ultrasonic treatment for 5 minutes;
iii) Incubating the polypeptide and oil at 50-60° C. with stirring at 20 rpm;
iv) Centrifuging the oil and the polypeptide at 700 g at 85° C. for 15 minutes.

The composition may in particular comprise a polypeptide, which has phospholipase C activity is derived from a fungus, and is selected from the group consisting of:

(a) a polypeptide having at least 60% sequence identity to the mature polypeptide of any one of SEQ ID NO: 2, SEQ ID NO: 4, and SEQ ID NO: 7;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 1, SEQ ID NO: 3 and SEQ ID NO: 5 (ii) the genomic DNA sequence comprising the mature polypeptide coding sequence of any one of SEQ ID NO: 1, SEQ ID NO: 3 and SEQ ID NO: 5, (iii) the cDNA sequence of SEQ ID NO: 5 (SEQ ID NO: 6) or (iv) the full-length complementary strand of (i), (ii) or (iii);

(c) a polypeptide encoded by a polynucleotide having at least 60% sequence identity to the mature polypeptide coding sequence of any one of SEQ ID NO: 1, SEQ ID NO: 3 and SEQ ID NO: 5;

(d) a variant of the mature polypeptide of any one of SEQ ID NO: 2, SEQ ID NO: 4, and SEQ ID NO: 7 comprising a substitution, deletion, and/or insertion at one or more positions; and
(e) a fragment of the polypeptide of (a), (b), (c), or (d).

In specific embodiments, the composition according to the invention comprises a polypeptide, which has phospholipase C activity and is selected from the group consisting of:

(a) a polypeptide having at least 60% sequence identity to the mature polypeptide of SEQ ID NO: 2,

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, (ii) the genomic DNA sequence comprising the mature polypeptide coding sequence of SEQ ID NO: 1, or (iii) the full-length complementary strand of (i) or (ii));

(c) a polypeptide encoded by a polynucleotide having at least 60% sequence identity to the mature polypeptide coding sequence of SEQ ID NO: 1;

(d) a variant of the mature polypeptide of SEQ ID NO: 2, comprising a substitution, deletion, and/or insertion at one or more positions; and

(e) a fragment of the polypeptide of (a), (b), (c), or (d).

In other specific embodiments, the composition comprises a polypeptide, which has phospholipase C activity and is selected from the group consisting of:

(a) a polypeptide having at least 60% sequence identity to the mature polypeptide of SEQ ID NO: 4;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 3 (ii) the genomic DNA sequence comprising the mature polypeptide coding sequence of SEQ ID NO: 3, or (iii) the full-length complementary strand of (i) or (ii);

(c) a polypeptide encoded by a polynucleotide having at least 60% sequence identity to the mature polypeptide coding sequence of SEQ ID NO: 3;

(d) a variant of the mature polypeptide of SEQ ID NO: 4, comprising a substitution, deletion, and/or insertion at one or more positions; and

(e) a fragment of the polypeptide of (a), (b), (c), or (d).

In still other embodiments, the composition comprises a polypeptide, which has phospholipase C activity and is selected from the group consisting of:

(a) a polypeptide having at least 60% sequence identity to the mature polypeptide of SEQ ID NO: 7;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 5 (ii) the genomic DNA sequence comprising the mature polypeptide coding sequence of SEQ ID NO: 5, (iii) the cDNA sequence of SEQ ID NO: 5 (SEQ ID NO: 6) or (iv) the full-length complementary strand of (i), (ii) or (iii);

(c) a polypeptide encoded by a polynucleotide having at least 60% sequence identity to the mature polypeptide coding sequence of SEQ ID NO: 5;

(d) a variant of the mature polypeptide of SEQ ID NO: 7 comprising a substitution, deletion, and/or insertion at one or more positions; and

(e) a fragment of the polypeptide of (a), (b), (c), or (d).

The said mature polypeptide may in particular consist of, essentially consist of or comprise an amino acid sequence selected from the group consisting of amino acid residues 19-643 of SEQ ID NO.: 2, amino acid residues 17-610 of SEQ ID NO.: 4, amino acid residues 20-626 of SEQ ID NO.: 7, and amino acid residues 37-626 of SEQ ID NO.: 7.

In other embodiments, the mature polypeptide of SEQ ID NO: 2 comprises, consists of or consists essentially of amino acids 43-618 of SEQ ID NO: 2. In further embodiments, the mature polypeptide of SEQ ID NO: 4 comprises, consists of or consists essentially of amino acids 20-576 of SEQ ID NO: 4. In still further embodiments of the invention, the mature polypeptide of SEQ ID NO: 7 comprises, consists of or consists essentially of amino acids 37-618 of SEQ ID NO: 7.

In further embodiments, the composition comprises a polypeptide, which has phospholipase C activity, is derived from a bacterium and is selected from the group consisting of:

(a) a polypeptide having at least 60% sequence identity to the mature polypeptide of any one of SEQ ID NO: 9, SEQ ID NO: 12, SEQ ID NO: 15, SEQ ID NO: 18, SEQ ID NO: 21, SEQ ID NO: 24, SEQ ID NO: 26, SEQ ID NO: 29, SEQ ID NO: 31, SEQ ID NO: 34, SEQ ID NO: 36, SEQ ID NO: 39, SEQ ID NO: 41, SEQ ID NO: 43 and SEQ ID NO: 45;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 8, SEQ ID NO: 11, SEQ ID NO: 14, SEQ ID NO: 17, SEQ ID NO: 20, SEQ ID NO: 23 SEQ ID NO: 25, SEQ ID NO: 28, SEQ ID NO: 30, SEQ ID NO: 33, SEQ ID NO: 35, SEQ ID NO: 38, SEQ ID NO: 40, SEQ ID NO: 42, and SEQ ID NO: 44 (ii) the genomic DNA sequence comprising the mature polypeptide coding sequence of any one of SEQ ID NO: 8, SEQ ID NO: 11, SEQ ID NO: 14, SEQ ID NO: 17, SEQ ID NO: 20, SEQ ID NO: 23, SEQ ID NO: 25, SEQ ID NO: 28, SEQ ID NO: 30, SEQ ID NO: 33, SEQ ID NO: 35, SEQ ID NO: 38, SEQ ID NO: 40, SEQ ID NO: 42, and SEQ ID NO: 44 or (iii) the full-length complementary strand of (i) or (ii);

(c) a polypeptide encoded by a polynucleotide having at least 60% sequence identity to the mature polypeptide coding sequence of any one of SEQ ID NO: 8, SEQ ID NO: 11, SEQ ID NO: 14, SEQ ID NO: 17, SEQ ID NO: 20, SEQ ID NO: 23, SEQ ID NO: 25, SEQ ID NO: 28, SEQ ID NO: 30, SEQ ID NO: 33, SEQ ID NO: 35, SEQ ID NO: 38, SEQ ID NO: 40 SEQ ID NO: 42 and SEQ ID NO: 44;

(d) a variant of the mature polypeptide of any one of SEQ ID NO: 9, SEQ ID NO: 12, SEQ ID NO: 15, SEQ ID NO: 18, SEQ ID NO: 21, SEQ ID NO: 24, SEQ ID NO: 26, SEQ ID NO: 29, SEQ ID NO: 31, SEQ ID NO: 34, SEQ ID NO: 36, SEQ ID NO: 39, SEQ ID NO: 41, SEQ ID NO: 43 and SEQ ID NO: 45 comprising a substitution, deletion, and/or insertion at one or more positions; and

(e) a fragment of the polypeptide of (a), (b), (c), or (d).

In still further embodiments, the composition according to the invention comprises a polypeptide, which has phospholipase C activity and is selected from the group consisting of:

(a) a polypeptide having at least 60% sequence identity to the mature polypeptide of any one of SEQ ID NO: 9, SEQ ID NO: 12, SEQ ID NO: 15, SEQ ID NO: 18, SEQ ID NO: 21, SEQ ID NO: 24, and SEQ ID NO: 43;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42 (ii) the genomic DNA sequence comprising the mature polypeptide coding sequence of any one of SEQ ID NO: 8, SEQ ID NO: 11, SEQ ID NO: 14, SEQ ID NO: 17, SEQ ID NO: 20, SEQ ID NO: 23, and SEQ ID NO: 42 or (iii) the full-length complementary strand of (i) or (ii);

(c) a polypeptide encoded by a polynucleotide having at least 60% sequence identity to the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42;

(d) a variant of the mature polypeptide of any one of SEQ ID NO: 9, SEQ ID NO: 12, SEQ ID NO: 15, SEQ ID NO: 18, SEQ ID NO: 21, SEQ ID NO: 24, and SEQ ID NO: 43 comprising a substitution, deletion, and/or insertion at one or more positions; and

(e) a fragment of the polypeptide of (a), (b), (c), or (d).

The composition according to the invention may, according to some embodiments comprise a polypeptide, which has phospholipase C activity and is selected from the group consisting of:

(a) a polypeptide having at least 60% sequence identity to the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 25, SEQ ID NO: 28, SEQ ID NO: 30, SEQ ID NO: 33, SEQ ID NO: 35, SEQ ID NO: 38, and SEQ ID NO: 44 (ii) the genomic DNA sequence comprising the mature polypeptide coding sequence of any one of SEQ ID NO: 25, SEQ ID NO: 28, SEQ ID NO: 30, SEQ ID NO: 33, SEQ ID NO: 35, SEQ ID NO: 38, and SEQ ID NO: 44 or (iii) the full-length complementary strand of (i) or (ii);

(c) a polypeptide encoded by a polynucleotide having at least 60% sequence identity to the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44;

(d) a variant of the mature polypeptide of any one of SEQ ID NO: 26, SEQ ID NO: 29, SEQ ID NO: 31, SEQ ID NO: 34, SEQ ID NO: 36 SEQ ID NO: 39, and SEQ ID NO: 45 comprising a substitution, deletion, and/or insertion at one or more positions; and

(e) a fragment of the polypeptide of (a), (b), (c), or (d).

In other embodiments, the composition according to the invention comprises a polypeptide, which has phospholipase C activity and is selected from the group consisting of:

(a) a polypeptide having at least 60% sequence identity to the mature polypeptide of SEQ ID NO: 41;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 40 (ii) the genomic DNA sequence comprising the mature polypeptide coding sequence of SEQ ID NO: 40, or (iii) the full-length complementary strand of (i) or (ii);

(c) a polypeptide encoded by a polynucleotide having at least 60% sequence identity to the mature polypeptide coding sequence of SEQ ID NO: 40;

(d) a variant of the mature polypeptide of SEQ ID NO: 41, comprising a substitution, deletion, and/or insertion at one or more positions; and

(e) a fragment of the polypeptide of (a), (b), (c), or (d).

According to the embodiments set forth above, the said mature polypeptide consists of or comprises an amino acid sequence selected from the group consisting of amino acid residues 23-322 of SEQ ID NO: 9, amino acid residues 24-322 of SEQ ID NO: 9, amino acid residues 25-322 of SEQ ID NO: 9, amino acid residues 26-322 of SEQ ID NO: 9, amino acid residues 27-322 of SEQ ID NO: 9, amino acid residues 28-322 of SEQ ID NO: 9, the amino acid sequence of SEQ ID NO: 10, amino acid residues 26-323 of SEQ ID NO: 12, the amino acid sequence of SEQ ID NO: 13, amino acid residues 26-323 of SEQ ID NO: 15, the amino acid sequence of SEQ ID NO: 16, amino acid residues 29-323 of SEQ ID NO: 18, the amino acid sequence of SEQ ID NO: 19, amino acid residues 26-322 of SEQ ID NO: 21, the amino acid sequence of SEQ ID NO: 22, amino acid residues 26-322 of SEQ ID NO: 24, amino acid residues 34-278 of SEQ ID NO: 26, the amino acid sequence of SEQ ID NO: 27, amino acid residues 29-283 of SEQ ID NO: 29, amino acid residues 25-283 of SEQ ID NO: 31, the amino acid sequence of SEQ ID NO: 32, amino acid residues 25-283 of SEQ ID NO: 34, amino acid residues 28-289 of SEQ ID NO: 36, the amino acid sequence of SEQ ID NO: 37, the amino acid sequence of SEQ ID NO: 39, amino acid residues 24-328 of SEQ ID NO: 41, amino acids 28-339 of SEQ ID NO: 43 and amino acids 25-280 of SEQ ID NO: 45.

Further, the composition according to the invention may be one, wherein said plurality of polypeptides having phospholipase C activity is selected from the group consisting of:
one polypeptide, which is as defined above and is derived from a fungus and one polypeptide, which is as defined above and is derived from a bacterium;
one polypeptide, which is as defined above and is derived from a fungus and two polypeptides, which are as defined above and are each derived from a bacterium;
one polypeptide, which is as defined above and is derived from a fungus and three polypeptides, which are as defined above and are each derived from a bacterium;
two polypeptides, which are as defined above and are each derived from a fungus and one polypeptide, which is as defined above and is derived from a bacterium;
two polypeptides, which are as defined above and are each derived from a fungus and two polypeptides, which are as defined above and are each derived from a bacterium;
two polypeptides, which are as defined above and are each derived from a fungus and three polypeptides, which are as defined above and are each derived from a bacterium;
three polypeptides, which are as defined above and are each derived from a fungus and one polypeptide, which is as defined above and is derived from a bacterium;
three polypeptides, which are as defined above and are each derived from a fungus and two polypeptides, which are as defined above and are each derived from a bacterium;
three polypeptides, which are as defined above and are each derived from a fungus and three polypeptides, which are as defined above and are each derived from a bacterium.

In embodiments relating to specific combinations of polypeptides, the composition comprises a polypeptide, which has phospholipase C activity, is derived from a fungus and is selected from the group consisting of:

(a) a polypeptide having at least 60% sequence identity to the mature polypeptide of SEQ ID NO: 2,

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, (ii) the genomic DNA sequence comprising the mature polypeptide coding sequence of any one of SEQ ID NO: 1, or (iii) the full-length complementary strand of (i) or (ii);

(c) a polypeptide encoded by a polynucleotide having at least 60% sequence identity to the mature polypeptide coding sequence of SEQ ID NO: 1;

(d) a variant of the mature polypeptide of SEQ ID NO: 2, comprising a substitution, deletion, and/or insertion at one or more positions; and

(e) a fragment of the polypeptide of (a), (b), (c), or (d);

and

a polypeptide, which has phospholipase C activity, is derived from a bacterium and is selected from the group consisting of:

(f) a polypeptide having at least 60% sequence identity to the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44 (ii) the genomic DNA sequence comprising the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44, or (iii) the full-length complementary strand of (i) or (ii);

(h) a polypeptide encoded by a polynucleotide having at least 60% sequence identity to the mature polypeptide coding sequence of any one of SEQ ID NO: 25, SEQ ID NO: 28, SEQ ID NO: 30, SEQ ID NO: 33, SEQ ID NO: 35, SEQ ID NO: 38, SEQ ID NO: 40 and SEQ ID NO: 44;

(i) a variant of the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45, comprising a substitution, deletion, and/or insertion at one or more positions; and

(j) a fragment of the polypeptide of (f), (g), (h), or (i).

In further embodiments relating to specific combinations of polypeptides, the composition comprises a polypeptide, which has phospholipase C activity, is derived from a fungus and is selected from the group consisting of:

(a) a polypeptide having at least 60% sequence identity to the mature polypeptide of SEQ ID NO: 2,

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, (ii) the genomic DNA sequence comprising the mature polypeptide coding sequence of any one of SEQ ID NO: 1, or (iii) the full-length complementary strand of (i) or (ii);

(c) a polypeptide encoded by a polynucleotide having at least 60% sequence identity to the mature polypeptide coding sequence of SEQ ID NO: 1;

(d) a variant of the mature polypeptide of SEQ ID NO: 2, comprising a substitution, deletion, and/or insertion at one or more positions; and

(e) a fragment of the polypeptide of (a), (b), (c), or (d);

and

a polypeptide, which has phospholipase C activity, is derived from a bacterium and is selected from the group consisting of:

(f) a polypeptide having at least 60% sequence identity to the mature polypeptide of SEQ ID NO: 39;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 38 (ii) the genomic DNA sequence comprising the mature polypeptide coding sequence of SEQ ID NO: 38, or (iii) the full-length complementary strand of (i) or (ii);

(h) a polypeptide encoded by a polynucleotide having at least 60% sequence identity to the mature polypeptide coding sequence of SEQ ID NO: 38;

(i) a variant of the mature polypeptide of SEQ ID NO: 39, comprising a substitution, deletion, and/or insertion at one or more positions; and

(j) a fragment of the polypeptide of (f), (g), (h), or (i).

In other embodiments relating to specific combinations of polypeptides, the composition comprises a polypeptide, which has phospholipase C activity, is derived from a fungus and is selected from the group consisting of:

(a) a polypeptide having at least 60% sequence identity to the mature polypeptide of SEQ ID NO: 4,

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 3, (ii) the genomic DNA sequence comprising the mature polypeptide coding sequence of any one of SEQ ID NO: 3, or (iii) the full-length complementary strand of (i) or (ii);

(c) a polypeptide encoded by a polynucleotide having at least 60% sequence identity to the mature polypeptide coding sequence of SEQ ID NO: 3;

(d) a variant of the mature polypeptide of SEQ ID NO: 4, comprising a substitution, deletion, and/or insertion at one or more positions; and

(e) a fragment of the polypeptide of (a), (b), (c), or (d);

and

a polypeptide, which has phospholipase C activity, is derived from a bacterium and is selected from the group consisting of:

(f) a polypeptide having at least 60% sequence identity to the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44 (ii) the genomic DNA sequence comprising the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44, or (iii) the full-length complementary strand of (i) or (ii);

(h) a polypeptide encoded by a polynucleotide having at least 60% sequence identity to the mature polypeptide coding sequence of any one of SEQ ID NO: 25, SEQ ID NO: 28, SEQ ID NO: 30, SEQ ID NO: 33, SEQ ID NO: 35, SEQ ID NO: 38, SEQ ID NO: 40 and SEQ ID NO: 44;

(i) a variant of the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45, comprising a substitution, deletion, and/or insertion at one or more positions; and

(j) a fragment of the polypeptide of (f), (g), (h), or (i).

In other embodiments relating to specific combinations of polypeptides, the composition comprises a polypeptide, which has phospholipase C activity, is derived from a fungus and is selected from the group consisting of:

(a) a polypeptide having at least 60% sequence identity to the mature polypeptide of SEQ ID NO: 4,

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 3, (ii) the genomic DNA sequence comprising the mature polypeptide coding sequence of any one of SEQ ID NO: 3, or (iii) the full-length complementary strand of (i) or (ii);

(c) a polypeptide encoded by a polynucleotide having at least 60% sequence identity to the mature polypeptide coding sequence of SEQ ID NO: 3;

(d) a variant of the mature polypeptide of SEQ ID NO: 4, comprising a substitution, deletion, and/or insertion at one or more positions; and

(e) a fragment of the polypeptide of (a), (b), (c), or (d);

and

a polypeptide, which has phospholipase C activity, is derived from a bacterium and is selected from the group consisting of:

(f) a polypeptide having at least 60% sequence identity to the mature polypeptide of SEQ ID NO: 39;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 38 (ii) the genomic DNA sequence comprising the mature polypeptide coding sequence of SEQ ID NO: 38, or (iii) the full-length complementary strand of (i) or (ii);

(h) a polypeptide encoded by a polynucleotide having at least 60% sequence identity to the mature polypeptide coding sequence of SEQ ID NO: 38;

(i) a variant of the mature polypeptide of SEQ ID NO: 39, comprising a substitution, deletion, and/or insertion at one or more positions; and

(j) a fragment of the polypeptide of (f), (g), (h), or (i).

In other embodiments relating to specific combinations of polypeptides, the compositions comprise a polypeptide, which has phospholipase C activity, is derived from a fungus, and is selected from the group consisting of:

(a) a polypeptide having at least 60% sequence identity to the mature polypeptide of SEQ ID NO: 7;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 5 (ii) the genomic DNA sequence comprising the mature polypeptide coding sequence of SEQ ID NO: 5, (iii) the cDNA sequence of SEQ ID NO: 5 (SEQ ID NO: 6) or (iv) the full-length complementary strand of (i), (ii) or (iii);

(c) a polypeptide encoded by a polynucleotide having at least 60% sequence identity to the mature polypeptide coding sequence of anyone of SEQ ID NO: 5;

(d) a variant of the mature polypeptide of SEQ ID NO: 7 comprising a substitution, deletion, and/or insertion at one or more positions; and

(e) a fragment of the polypeptide of (a), (b), (c), or (d);

and

a polypeptide, which has phospholipase C activity, is derived from a bacterium and is selected from the group consisting of:

(f) a polypeptide having at least 60% sequence identity to the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45;

(g) a polypeptide encoded by a polynucleotide that hybridizes under medium high stringency conditions with (i) the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44 (ii) the genomic DNA sequence comprising the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44, or (iii) the full-length complementary strand of (i) or (ii);

(h) a polypeptide encoded by a polynucleotide having at least 60% sequence identity to the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44;

(i) a variant of the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45, comprising a substitution, deletion, and/or insertion at one or more positions; and

(j) a fragment of the polypeptide of (f), (g), (h), or (i).

In other embodiments relating to specific combinations of polypeptides, the compositions comprise a polypeptide, which has phospholipase C activity, is derived from a fungus, and is selected from the group consisting of:

(a) a polypeptide having at least 60% sequence identity to the mature polypeptide of SEQ ID NO: 7;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 5 (ii) the genomic DNA sequence comprising the mature polypeptide coding sequence of SEQ ID NO: 5, (iii) the cDNA sequence of SEQ ID NO: 5 (SEQ ID NO: 6) or (iv) the full-length complementary strand of (i), (ii) or (iii);

(c) a polypeptide encoded by a polynucleotide having at least 60% sequence identity to the mature polypeptide coding sequence of anyone of SEQ ID NO: 5;

(d) a variant of the mature polypeptide of SEQ ID NO: 7 comprising a substitution, deletion, and/or insertion at one or more positions; and

(e) a fragment of the polypeptide of (a), (b), (c), or (d);

and

a polypeptide, which has phospholipase C activity, is derived from a bacterium and is selected from the group consisting of:

(f) a polypeptide having at least 60% sequence identity to the mature polypeptide of SEQ ID NO: 39;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 38 (ii) the genomic DNA sequence comprising the mature polypeptide coding sequence of SEQ ID NO: 38, or (iii) the full-length complementary strand of (i) or (ii);

(h) a polypeptide encoded by a polynucleotide having at least 60% sequence identity to the mature polypeptide coding sequence of SEQ ID NO: 38;

(i) a variant of the mature polypeptide of SEQ ID NO: 39, comprising a substitution, deletion, and/or insertion at one or more positions; and

(j) a fragment of the polypeptide of (f), (g), (h), or (i).

In still other embodiments relating to specific combinations of polypeptides, the composition comprises a polypeptide which has phospholipase C activity, is derived from a fungus and is selected from the group consisting of:

(a) a polypeptide having at least 60% sequence identity to the mature polypeptide of SEQ ID NO: 7;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 5 (ii) the genomic DNA sequence comprising the mature polypeptide coding sequence of SEQ ID NO: 5, (iii) the cDNA sequence of SEQ ID NO: 5 (SEQ ID NO: 6) or (iv) the full-length complementary strand of (i), (ii) or (iii);

(c) a polypeptide encoded by a polynucleotide having at least 60% sequence identity to the mature polypeptide coding sequence of anyone of SEQ ID NO: 5;

(d) a variant of the mature polypeptide of SEQ ID NO: 7 comprising a substitution, deletion, and/or insertion at one or more positions; and

(e) a fragment of the polypeptide of (a), (b), (c), or (d);

and

a polypeptide, which has phospholipase C activity, is derived from a bacterium and is selected from the group consisting of:

(f) a polypeptide having at least 60% sequence identity to the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 43;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42, (ii) the genomic DNA sequence comprising the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42, or (iii) the full-length complementary strand of (i) or (ii);

(h) a polypeptide encoded by a polynucleotide having at least 60% sequence identity to the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42;

(i) a variant of the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 43, comprising a substitution, deletion, and/or insertion at one or more positions; and

(j) a fragment of the polypeptide of (f), (g), (h), or (i);

and

a polypeptide, which has phospholipase C activity, is derived from a bacterium and is selected from the group consisting of:

(k) a polypeptide having at least 60% sequence identity to the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45;

(l) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44 (ii) the genomic DNA sequence comprising the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44, or (iii) the full-length complementary strand of (i) or (ii);

(m) a polypeptide encoded by a polynucleotide having at least 60% sequence identity to the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44;

(n) a variant of the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45, comprising a substitution, deletion, and/or insertion at one or more positions; and

(o) a fragment of the polypeptide of (k), (l), (m), or (n).

In still other embodiments relating to specific combinations of polypeptides, the composition comprises a polypeptide which has phospholipase C activity, is derived from a fungus and is selected from the group consisting of:

(a) a polypeptide having at least 60% sequence identity to the mature polypeptide of SEQ ID NO: 7;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 5 (ii) the genomic DNA sequence comprising the mature polypeptide coding sequence of SEQ ID NO: 5, (iii) the cDNA sequence of SEQ ID NO: 5 (SEQ ID NO: 6) or (iv) the full-length complementary strand of (i), (ii) or (iii);

(c) a polypeptide encoded by a polynucleotide having at least 60% sequence identity to the mature polypeptide coding sequence of anyone of SEQ ID NO: 5;

(d) a variant of the mature polypeptide of SEQ ID NO: 7 comprising a substitution, deletion, and/or insertion at one or more positions; and

(e) a fragment of the polypeptide of (a), (b), (c), or (d);

and

a polypeptide, which has phospholipase C activity, is derived from a bacterium and is selected from the group consisting of:

(f) a polypeptide having at least 60% sequence identity to the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 43;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42, (ii) the genomic DNA sequence comprising the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42, or (iii) the full-length complementary strand of (i) or (ii);

(h) a polypeptide encoded by a polynucleotide having at least 60% sequence identity to the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42;

(i) a variant of the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 43, comprising a substitution, deletion, and/or insertion at one or more positions; and

(j) a fragment of the polypeptide of (f), (g), (h), or (i);

and

a polypeptide, which has phospholipase C activity, is derived from a bacterium and is selected from the group consisting of:

(k) a polypeptide having at least 60% sequence identity to the mature polypeptide of SEQ ID NO: 39;

(l) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 38 (ii) the genomic DNA sequence comprising the mature polypeptide coding sequence of SEQ ID NO: 38, or (iii) the full-length complementary strand of (i) or (ii);

(m) a polypeptide encoded by a polynucleotide having at least 60% sequence identity to the mature polypeptide coding sequence of SEQ ID NO: 38;

(n) a variant of the mature polypeptide of SEQ ID NO: 39, comprising a substitution, deletion, and/or insertion at one or more positions; and

(o) a fragment of the polypeptide of (k), (l), (m), or (n).

In even further embodiments relating to specific combinations of polypeptides, the composition comprises a polypeptide, which has phospholipase C activity and is derived from a fungus, said polypeptide being selected from the group consisting of:

(a) a polypeptide having at least 60% sequence identity to the mature polypeptide of any one of SEQ ID NO: 2,

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, (ii) the genomic DNA sequence comprising the mature polypeptide coding sequence of any one of SEQ ID NO: 1, or (iii) the full-length complementary strand of (i) or (ii);

(c) a polypeptide encoded by a polynucleotide having at least 60% sequence identity to the mature polypeptide coding sequence of SEQ ID NO: 1;

(d) a variant of the mature polypeptide of SEQ ID NO: 2, comprising a substitution, deletion, and/or insertion at one or more positions; and

(e) a fragment of the polypeptide of (a), (b), (c), or (d);

and

a polypeptide, which has phospholipase C activity, is derived from a bacterium, and is selected from the group consisting of:

(f) a polypeptide having at least 60% sequence identity to the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 43;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42, (ii) the genomic DNA sequence comprising the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42, or (iii) the full-length complementary strand of (i) or (ii);

(h) a polypeptide encoded by a polynucleotide having at least 60% sequence identity to the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42;

(i) a variant of the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 43, comprising a substitution, deletion, and/or insertion at one or more positions; and

(j) a fragment of the polypeptide of (f), (g), (h), or (i);

and

a polypeptide, which has phospholipase C activity, is derived from a bacterium is selected from the group consisting of:

(k) a polypeptide having at least 60% sequence identity to the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45;

(l) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44 (ii) the genomic DNA sequence comprising the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44, or (iii) the full-length complementary strand of (i) or (ii);

(m) a polypeptide encoded by a polynucleotide having at least 60% sequence identity to the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44;

(n) a variant of the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45, comprising a substitution, deletion, and/or insertion at one or more positions; and

(o) a fragment of the polypeptide of (k), (l), (m), or (n).

Particular embodiments of the invention provides a composition, which comprises at least one polypeptide, which has phospholipase C activity and is derived from a fungus; and at least one polypeptide selected from the group consisting of:

(a) a polypeptide having at least 60% sequence identity to the mature polypeptide of any one of SEQ ID NO: 41;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 40 (ii) the genomic DNA sequence comprising the mature polypeptide coding sequence of any one of SEQ ID NO: 40, (iii) the cDNA sequence of SEQ ID NO: 40 or (iv) the full-length complementary strand of (i), (ii) or (iii);

(c) a polypeptide encoded by a polynucleotide having at least 60% sequence identity to the mature polypeptide coding sequence of SEQ ID NO: 40;

(d) a variant of the mature polypeptide of SEQ ID NO: 41 comprising a substitution, deletion, and/or insertion at one or more positions; and

(e) a fragment of the polypeptide of (a), (b), (c), or (d).

In even further embodiments relating to specific combinations of polypeptides, the composition comprises a polypeptide, which has phospholipase C activity and is derived from a fungus, said polypeptide being selected from the group consisting of:

(a) a polypeptide having at least 60% sequence identity to the mature polypeptide of any one of SEQ ID NO: 2,

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, (ii) the genomic DNA sequence comprising the mature polypeptide coding sequence of any one of SEQ ID NO: 1, or (iii) the full-length complementary strand of (i) or (ii);

(c) a polypeptide encoded by a polynucleotide having at least 60% sequence identity to the mature polypeptide coding sequence of SEQ ID NO: 1;

(d) a variant of the mature polypeptide of SEQ ID NO: 2, comprising a substitution, deletion, and/or insertion at one or more positions; and

(e) a fragment of the polypeptide of (a), (b), (c), or (d);

and

a polypeptide, which has phospholipase C activity, is derived from a bacterium, and is selected from the group consisting of:

(f) a polypeptide having at least 60% sequence identity to the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 43;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42, (ii) the genomic DNA sequence comprising the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42, or (iii) the full-length complementary strand of (i) or (ii);

(h) a polypeptide encoded by a polynucleotide having at least 60% sequence identity to the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42;

(i) a variant of the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 43, comprising a substitution, deletion, and/or insertion at one or more positions; and

(j) a fragment of the polypeptide of (f), (g), (h), or (i);

and

a polypeptide, which has phospholipase C activity, is derived from a bacterium is selected from the group consisting of:

(k) a polypeptide having at least 60% sequence identity to the mature polypeptide of SEQ ID NO: 39;

(l) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 38 (ii) the genomic DNA sequence comprising the mature polypeptide coding sequence of SEQ ID NO: 38, or (iii) the full-length complementary strand of (i) or (ii);

(m) a polypeptide encoded by a polynucleotide having at least 60% sequence identity to the mature polypeptide coding sequence of SEQ ID NO: 38;

(n) a variant of the mature polypeptide of SEQ ID NO: 39, comprising a substitution, deletion, and/or insertion at one or more positions; and

(o) a fragment of the polypeptide of (k), (l), (m), or (n).

It will be understood that this composition may have any of the characteristics and features set forth in relation to the other compositions disclosed above.

Also, the said mature polypeptide may comprise or consists of amino acid residues 24-328 of SEQ ID NO: 41 and/or the said variant in (d) may comprise amino acid residues 56-195 of SEQ ID NO: 41.

The said fragment in item (e) above may in some embodiments comprise amino acid residues 56-195 of SEQ ID NO: 41.

It is further contemplated that the composition according to the invention comprises one or more polypeptides selected from the group consisting of a phospholipase A1, a phospholipase A2, a phospholipase B, a phospholipase D and a protease, an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, or xylanase

In relation to items (a), (f) and/or (k) as defined in each of the aspects and embodiments set forth above, the a polypeptide having at least 65% sequence identity to the mature polypeptide defined by any one of the sequence identifiers provided in item (a) of the particular embodiment, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100%. Further, in relation to each of the above embodiments, the polypeptide may have been isolated.

According to each of the aspects and embodiments of the invention, the variants of each mature polypeptide may comprise a substitution, deletion, and/or insertion at one or more (e.g., several) positions. In an embodiment, the number of amino acid substitutions, deletions and/or insertions introduced into the mature polypeptide of SEQ ID NO: 2 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

As the skilled person will know, essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for Phospholipase C activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

According to each aspect and embodiment of the invention, the polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

In the embodiments of the invention provided above, the polypeptide having at least 60% sequence identity to the mature polypeptide of any one of SEQ ID NO: 2, SEQ ID NO: 4, and SEQ ID NO: 7 preferably has a length of 540-640 amino acid residues, such as a length of, 550-640 amino acid residues, 560-640 amino acid residues, 570-640 amino acid residues, 540-630 amino acid residues, such as a length of, 550-630 amino acid residues, 560-630 amino acid residues, 570-630 amino acid residues, 570-620 amino acid residues, 570-610 amino acid residues, 570-600 amino acid residues 570-597 amino acid residues, 570-596 amino acid residues, 580-640 amino acid residues, 540-630 amino acid residues, such as a length of, 540-620 amino acid residues, 540-610 amino acid residues, 540-600 amino acid residues, 540-590 amino acid residues, 540-580 amino acid residues, 540-570 amino acid residues, 540-560 amino acid residues, 540-560 amino acid residues, 550-630 amino acid residues, such as a length of, 550-620 amino acid residues, 550-610 amino acid residues, 550-600 amino acid residues, 550-590 amino acid residues, 550-580 amino acid residues, 550-570 amino acid residues, 550-560 amino acid residues, 550-560 amino acid residues, 560-630 amino acid residues, such as a length of, 560-620 amino acid residues, 560-610 amino acid residues, 560-600 amino acid residues, 560-590 amino acid residues, 560-580 amino acid residues, 560-570 amino acid residues, 560-560 amino acid residues, 560-560 amino acid residues, 570-630 amino acid residues, such as a length of, 570-620 amino acid residues, 570-610 amino acid residues, 570-600 amino acid residues, 570-590 amino acid residues, 570-580 amino acid residues, 570-570 amino acid residues, 570-560 amino acid residues, 570-560 amino acid residues, 580-630 amino acid residues, 580-620 amino acid residues, 580-610 amino acid residues, 580-600 amino acid residues, 580-598 amino acid residues, 580-597 amino acid residues, 580-596 amino acid residues, 590-640 amino acid residues, 590-630 amino acid residues, 590-620 amino acid residues, 590-610 amino acid residues, 590-600 amino acid residues, 590-698 amino acid residues, 590-597 amino acid residues, 590-596 amino acid residues, 595-640 amino acid residues, 595-630 amino acid residues, 595-620 amino acid residues, 595-610 amino acid residues, 595-600 amino acid residues, 595-598 amino acid residues, 595-597 amino acid residues, or a length of 595-596 amino acid residues.

Also, in the embodiments of the invention provided above, the polypeptide having at least 60% sequence identity to the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 43 preferably has a length of 280-320 amino acid residues, such as a length of 280-310 amino acid residues, 280-305 amino acid residues, 280-300 amino acid residues, 280-298 amino acid residues 280-297 amino acid residues, 280-296 amino acid residues, 285-320 amino acid residues, 285-315 amino acid residues, 285-310 amino acid residues, 285-305 amino acid residues, 285-300 amino acid residues, 285-298 amino acid residues, 285-297 amino acid residues, 285-296 amino acid residues, 290-320 amino acid residues, 290-315 amino acid residues, 290-310 amino acid residues, 290-305 amino acid residues, 290-300 amino acid residues, 290-298 amino acid residues, 290-297 amino acid residues, 290-296 amino acid residues, 295-320 amino acid residues, 295-315 amino acid residues, 295-310 amino acid residues, 295-305 amino acid residues, 295-300 amino acid residues, 295-298 amino acid residues, 255-297 amino acid residues, or a length of 295-296 amino acid residues.

Similarly, the polypeptide having at least 60% sequence identity to the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45 preferably has a length of 220-280 amino acid residues, such as a length of 220-270 amino acid residues, 220-260 amino acid residues, 220-250 amino acid residues, 220-248 amino acid residues 220-246 amino acid residues, 220-244 amino acid residues, 225-280 amino acid residues, 225-270 amino acid residues, 225-260 amino acid residues, 225-250 amino acid residues, 225-248 amino acid residues 225-246 amino acid residues, 225-244 amino acid residues, 230-280 amino acid residues, 230-270 amino acid residues, 230-260 amino acid residues, 230-250 amino acid residues, 230-248 amino acid residues, 230-246 amino acid residues, 230-244 amino acid residues, 235-280 amino acid residues, 235-270 amino acid residues, 235-260 amino acid residues, 235-250 amino acid residues, 235-248 amino acid residues 235-246 amino acid residues, 235-244 amino acid residues, 240-280 amino acid residues, 240-270 amino acid residues, 240-260 amino acid residues, 240-250 amino acid residues, 240-248 amino acid residues 240-246 amino acid residues, 240-244 amino acid residues, 242-280 amino acid residues, 242-270 amino acid residues, 242-260 amino acid residues, 242-250 amino acid residues, 242-248 amino acid residues 242-246 amino acid residues, 242-244 amino acid residues, 243-280 amino acid residues, 243-270 amino acid residues, 243-260 amino acid residues, 243-250 amino acid residues, 243-248 amino acid residues 243-246 amino acid residues, 243-244 amino acid residues,

In further embodiments of the invention the composition comprises one or more enzyme activities in addition to the said plurality of polypeptides having phospholipase C activity. In particular, the composition may comprise one or more polypeptides having phopolipase A activity. The one or more polypeptides having phospholipase A activity may in particular be an isolated polypeptide having phospholipase A activity, selected from the group consisting of:

(a) a polypeptide having at least 80% sequence identity, such as at least 85%, at least 90%, at least 95% or at least 98% sequence identity, to the mature polypeptide of SEQ ID NO: 46 or to the polypeptide of SEQ ID NO: 47;

(b) a fragment of the polypeptide of (a) that has phospholipase A activity.

The compositions according to the invention may be prepared in accordance with methods known in the art and may be in the form of a liquid or a dry composition. The compositions may be stabilized in accordance with methods known in the art.

### Uses

The phospholipases or compositions of the invention may be applied in a process for removing phospholipids from oil, e.g. a vegetable oil, animal oil or fat, tallow, or grease; i.e., in a process to reduce the phospholipid content in the oil. The degumming process is applicable to the purification of any edible oil which contains phospholipid, e.g., vegetable oil such as soybean oil, rape seed oil, or sunflower oil or any other oil mentioned under the definition of crude oils.

Applications in which the phospholipase of the invention can be used comprise i) degumming of oil, e.g. vegetable oil, or an edible vegetable oil, or in a process comprising hydrolysis of phospholipids in the gum fraction from water degumming to release entrapped triglyceride oil, ii) in a process comprising hydrolysis of phospholipids to obtain improved phospholipid emulsifiers, in particular wherein said phospholipid is lecithin, iii) in a process for improving the filterability of an aqueous solution or slurry of carbohydrate origin which contains phospholipid, iv) in a process for the extraction of oil, v) in a process for the production of an animal feed product, vi) in a process for the production of a biofuel, e.g. a biodiesel, vii) in a process for the production of a detergent product, and/or viii) in a process for making a baked product, comprising adding the phospholipase to a dough, and baking the dough to make the baked product. Hence, another aspect of the present invention provides a composition as defined in any of the aspects and embodiments above in any of claim for use in the afore-mentioned applications.

### Processes

The combinations of phospholipases set forth above and the compositions of the invention may be applied in a process comprising contacting a phospholipid or lysophospholipid with a combination of fungal and bacterial PLCs, such as the combinations provided in the compositions of the invention. The phospholipases in the compositions react with the phospholipids or lysophospholipid to form monoglyceride or diglyceride and a phosphate ester or phosphoric acid.

Hence, in another aspect, the invention provides a process for hydrolysing a phospholipid or lysophospholipid, comprising contacting said phospholipid or lysophospholipid with a plurality of polypeptides having phospholipase C activity, wherein
at least one of said polypeptides having phospholipase C activity is a polypeptide derived from a fungus; and
at least one of said polypeptides having phospholipase C activity is a polypeptide derived from a bacterium.

According to some embodiments, the process comprises contacting said phospholipid or lysophospholipid with a polypeptide, which has phospholipase C activity, is derived from a fungus and is selected from the group consisting of:

(a) a polypeptide having at least 60% sequence identity to the mature polypeptide of any one of SEQ ID NO: 2, SEQ ID NO: 4, and SEQ ID NO: 7;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 1, SEQ ID NO: 3 and SEQ ID NO: 5 (ii) the genomic DNA sequence comprising the mature polypeptide coding sequence of any one of SEQ ID NO: 1, SEQ ID NO: 3 and SEQ ID NO: 5, (iii) the cDNA sequence of SEQ ID NO: 5 (SEQ ID NO: 6) or (iv) the full-length complementary strand of (i), (ii) or (iii);

(c) a polypeptide encoded by a polynucleotide having at least 60% sequence identity to the mature polypeptide coding sequence of anyone of SEQ ID NO: 1, SEQ ID NO: 3 and SEQ ID NO: 5;

(d) a variant of the mature polypeptide of any one of SEQ ID NO: 2, SEQ ID NO: 4, and SEQ ID NO: 7 comprising a substitution, deletion, and/or insertion at one or more positions; and

(e) a fragment of the polypeptide of (a), (b), (c), or (d).

In other embodiments, the process comprises contacting said phospholipid or lysophospholipid with a polypeptide, which has phospholipase C activity and is selected from the group consisting of:

(a) a polypeptide having at least 60% sequence identity to the mature polypeptide of SEQ ID NO: 2,

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, (ii) the genomic DNA sequence comprising the mature polypeptide coding sequence of SEQ ID NO: 1, or (iii) the full-length complementary strand of (i) or (ii));

(c) a polypeptide encoded by a polynucleotide having at least 60% sequence identity to the mature polypeptide coding sequence of SEQ ID NO: 1,

(d) a variant of the mature polypeptide of SEQ ID NO: 2, comprising a substitution, deletion, and/or insertion at one or more positions; and

(e) a fragment of the polypeptide of (a), (b), (c), or (d).

The process may also comprise contacting said phospholipid or lysophospholipid with a polypeptide, which has phospholipase C activity and is selected from the group consisting of:

(a) a polypeptide having at least 60% sequence identity to the mature polypeptide of SEQ ID NO: 4;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 3 (ii) the genomic DNA sequence comprising the mature polypeptide coding sequence of SEQ ID NO: 3, or (iii) the full-length complementary strand of (i) or (ii);

(c) a polypeptide encoded by a polynucleotide having at least 60% sequence identity to the mature polypeptide coding sequence of SEQ ID NO: 3;

(d) a variant of the mature polypeptide of SEQ ID NO: 4, comprising a substitution, deletion, and/or insertion at one or more positions; and

(e) a fragment of the polypeptide of (a), (b), (c), or (d).

In other embodiments the process of the Invention comprises contacting said phospholipid or lysophospholipid with a polypeptide, which has phospholipase C activity and is selected from the group consisting of:

(a) a polypeptide having at least 60% sequence identity to the mature polypeptide of SEQ ID NO: 7;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 5 (ii) the genomic DNA sequence comprising the mature polypeptide coding sequence of SEQ ID NO: 5, (iii) the cDNA sequence of SEQ ID NO: 5 (SEQ ID NO: 6) or (iv) the full-length complementary strand of (i), (ii) or (iii);

(c) a polypeptide encoded by a polynucleotide having at least 60% sequence identity to the mature polypeptide coding sequence of SEQ ID NO: 5;

(d) a variant of the mature polypeptide of SEQ ID NO: 7 comprising a substitution, deletion, and/or insertion at one or more positions; and

(e) a fragment of the polypeptide of (a), (b), (c), or (d).

According to these embodiments as well as embodiments disclosed in the following, the said mature polypeptide may in particular consist of or comprise an amino acid sequence selected from the group consisting of amino acid residues 19-643 of SEQ ID NO.: 2, amino acid residues 17-610 of SEQ ID NO.: 4, amino acid residues 20-626 of SEQ ID NO.: 7, and amino acid residues 37-626 of SEQ ID NO: 7.

In other embodiments, the mature polypeptide of SEQ ID NO: 2 comprises, consists of or consists essentially of amino acids 43-618 of SEQ ID NO: 2. In further embodiments, the mature polypeptide of SEQ ID NO: 4 comprises, consists of or consists essentially of amino acids 20-576 of SEQ ID NO: 4. In still further embodiments of the invention, the mature polypeptide of SEQ ID NO: 7 comprises, consists of or consists essentially of amino acids 37-618 of SEQ ID NO: 7.

In further embodiments, the process according to the Invention comprises contacting said phospholipid or lysophospholipid with a polypeptide, which has phospholipase C activity, and is selected from the group consisting of:

(a) a polypeptide having at least 60% sequence identity to the mature polypeptide of any one of SEQ ID NO: 9, SEQ ID NO: 12, SEQ ID NO: 15, SEQ ID NO: 18, SEQ ID NO: 21, SEQ ID NO: 24, SEQ ID NO: 26, SEQ ID NO: 29, SEQ ID NO: 31, SEQ ID NO: 34, SEQ ID NO: 36, SEQ ID NO: 39, SEQ ID NO: 41, SEQ ID NO: 43 and SEQ ID NO: 45;

(b) a polypeptide encoded by a polynucleotide that hybridizes under medium high stringency conditions with (i) the mature polypeptide coding sequence of any one of SEQ ID NO: 8, SEQ ID NO: 11, SEQ ID NO: 14, SEQ ID NO: 17, SEQ ID NO: 20, SEQ ID NO: 23 SEQ ID NO: 25, SEQ ID NO: 28, SEQ ID NO: 30, SEQ ID NO: 33, SEQ ID NO: 35, SEQ ID NO: 38, SEQ ID NO: 40, SEQ ID NO: 42 and SEQ ID NO: 44 (ii) the genomic DNA sequence comprising the mature polypeptide coding sequence of any one of SEQ ID NO: 8, SEQ ID NO: 11, SEQ ID NO: 14, SEQ ID NO: 17, SEQ ID NO: 20, SEQ ID NO: 23, SEQ ID NO: 25, SEQ ID NO: 28, SEQ ID NO: 30, SEQ ID NO: 33, SEQ ID NO: 35, SEQ ID NO: 38, SEQ ID NO: 40, and SEQ ID NO: 42 and SEQ ID NO: 44 or (iii) the full-length complementary strand of (i) or (ii);

(c) a polypeptide encoded by a polynucleotide having at least 60% sequence identity to the mature polypeptide coding sequence of any one of SEQ ID NO: 8, SEQ ID NO: 11, SEQ ID NO: 14, SEQ ID NO: 17, SEQ ID NO: 20, SEQ ID NO: 23, SEQ ID NO: 25, SEQ ID NO: 28, SEQ ID NO: 30, SEQ ID NO: 33, SEQ ID NO: 35, SEQ ID NO: 38, SEQ ID NO: 40, SEQ ID NO: 42 and SEQ ID NO: 44;

(d) a variant of the mature polypeptide of any one of SEQ ID NO: 9, SEQ ID NO: 12, SEQ ID NO: 15, SEQ ID NO: 18, SEQ ID NO: 21, SEQ ID NO: 24, SEQ ID NO: 26, SEQ ID NO: 29, SEQ ID NO: 31, SEQ ID NO: 34, SEQ ID NO: 36, SEQ ID NO: 39, SEQ ID NO: 41, and SEQ ID NO: 43 and SEQ ID NO: 45 comprising a substitution, deletion, and/or insertion at one or more positions; and

(e) a fragment of the polypeptide of (a), (b), (c), or (d).

In still further embodiments the process according to the invention comprises contacting said phospholipid or lysophospholipid with a polypeptide, which has phospholipase C activity and is selected from the group consisting of:

(a) a polypeptide having at least 60% sequence identity to the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 43;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42 (ii) the genomic DNA sequence comprising the mature polypeptide coding sequence of any one of SEQ ID NO: 8, SEQ ID NO: 11, SEQ ID NO: 14, SEQ ID NO: 17, SEQ ID NO: 20, SEQ ID NO: 23, and SEQ ID NO: 42 or (iii) the full-length complementary strand of (i) or (ii);

(c) a polypeptide encoded by a polynucleotide having at least 60% sequence identity to the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42;

(d) a variant of the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 43, comprising a substitution, deletion, and/or insertion at one or more positions; and

(e) a fragment of the polypeptide of (a), (b), (c), or (d).

The process may also, according to other embodiments, comprise contacting said phospholipid or lysophospholipid with a polypeptide, which has phospholipase C activity and is selected from the group consisting of:

(a) a polypeptide having at least 60% sequence identity to the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44, (ii) the genomic DNA sequence comprising the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44, or (iii) the full-length complementary strand of (i) or (ii);

(c) a polypeptide encoded by a polynucleotide having at least 60% sequence identity to the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44;

(d) a variant of the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45, comprising a substitution, deletion, and/or insertion at one or more positions; and

(e) a fragment of the polypeptide of (a), (b), (c), or (d).

The process according to the invention may in further embodiments comprise contacting said phospholipid or lysophospholipid with a polypeptide, which has phospholipase C activity and is selected from the group consisting of:

(a) a polypeptide having at least 60% sequence identity to the mature polypeptide of SEQ ID NO: 41;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 40 (ii) the genomic DNA sequence comprising the mature polypeptide coding sequence of SEQ ID NO: 40, or (iii) the full-length complementary strand of (i) or (ii);

(c) a polypeptide encoded by a polynucleotide having at least 60% sequence identity to the mature polypeptide coding sequence of SEQ ID NO: 40;

(d) a variant of the mature polypeptide of SEQ ID NO: 41, comprising a substitution, deletion, and/or insertion at one or more positions; and

(e) a fragment of the polypeptide of (a), (b), (c), or (d).

(f) PURIFINE® PLC, which is commercially available from DSM.

Variants of the mature polypeptide of SEQ ID NO 41 and of PURIFINE® PLC are disclosed in international patent publication WO 2011/0146812. In particular, the variants of SEQ ID NO 41 may have one or more of the amino acid changes or substitutions described in Table 12 (page 210), Table 13 (pages 211-18), Table 14 (pages 220-21) and/or table 15 (pages 222-23) in the said WO-publication. The content of all four tables is incorporated herein by reference in its entirety.

In relation to the embodiments disclosed above, the said mature polypeptide may consist of, consist essentially of or comprise an amino acid sequence selected from the group consisting of amino acid residues 23-322 of SEQ ID NO: 9, amino acid residues 24-322 of SEQ ID NO: 9, amino acid residues 25-322 of SEQ ID NO: 9, amino acid residues 26-322 of SEQ ID NO: 9, amino acid residues 27-322 of SEQ ID NO: 9, amino acid residues 28-322 of SEQ ID NO: 9, the amino acid sequence of SEQ ID NO: 10, amino acid residues 26-323 of SEQ ID NO: 12, the amino acid sequence of SEQ ID NO: 13, amino acid residues 26-323 of SEQ ID NO: 15, the amino acid sequence of SEQ ID NO: 16, amino acid residues 29-323 of SEQ ID NO: 18, the amino acid sequence of SEQ ID NO: 19, amino acid residues 26-322 of SEQ ID NO: 21, the amino acid sequence of SEQ ID NO: 22, amino acid residues 26-322 of SEQ ID NO: 24, amino acid residues 34-278 of SEQ ID NO: 26, the amino acid sequence of SEQ ID NO: 27, amino acid residues 29-283 of SEQ ID NO: 29, amino acid residues 25-283 of SEQ ID NO: 31, the amino acid sequence of SEQ ID NO: 32, amino acid residues 25-283 of SEQ ID NO: 34, amino acid residues 28-289 of SEQ ID NO: 36, the amino acid sequence of SEQ ID NO: 37, the amino acid sequence of SEQ ID NO: 39, amino acid residues 24-328 of SEQ ID NO: 41, amino acid residues 28-339 of SEQ ID NO: 43, and amino acid residues 25-280 of SEQ ID NO: 45.

In particular, the said plurality of polypeptides having phospholipase C activity may be selected from the group consisting of:
one polypeptide which is as defined above and is derived from a fungus and one polypeptide which is as defined above and is derived from a bacterium;
one polypeptide which is as defined above and is derived from a fungus and two polypeptides which are as defined above and are each derived from a bacterium;
one polypeptide which is as defined above and is derived from a fungus and three polypeptides which are as defined above and are each derived from a bacterium;
two polypeptides which are as defined above and are each derived from a fungus and one polypeptide which is as defined above and is derived from a bacterium;
two polypeptides which are as defined above and are each derived from a fungus and two polypeptides which are as defined above and are each derived from a bacterium;
two polypeptides which are as defined above and are each derived from a fungus and three polypeptides which are as defined above and are each derived from a bacterium;
three polypeptides which are as defined above and are each derived from a fungus and one polypeptide which is as defined above and is derived from a bacterium;
three polypeptides which are as defined above and are each derived from a fungus and two polypeptides which are as defined above and are each derived from a bacterium;
three polypeptides which are as defined above and are each derived from a fungus and three polypeptides which are as defined above and are each derived from a bacterium.

In particular embodiments, the process according to the invention comprises contacting said phospholipid or lysophospholipid with a polypeptide, which has phospholipase C activity, and is selected from the group consisting of:

(a) a polypeptide having at least 60% sequence identity to the mature polypeptide of SEQ ID NO: 2,

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, (ii) the genomic DNA sequence comprising the mature polypeptide coding sequence of any one of SEQ ID NO: 1, or (iii) the full-length complementary strand of (i) or (ii);

(c) a polypeptide encoded by a polynucleotide having at least 60% sequence identity to the mature polypeptide coding sequence of SEQ ID NO: 1;

(d) a variant of the mature polypeptide of SEQ ID NO: 2, comprising a substitution, deletion, and/or insertion at one or more positions; and

(e) a fragment of the polypeptide of (a), (b), (c), or (d);

and
with a polypeptide, which has phospholipase C activity and is selected from the group consisting of:

(f) a polypeptide having at least 60% sequence identity to the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44 (ii) the genomic DNA sequence comprising the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44, or (iii) the full-length complementary strand of (i) or (ii);

(h) a polypeptide encoded by a polynucleotide having at least 60% sequence identity to the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 440;

(i) a variant of the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45, comprising a substitution, deletion, and/or insertion at one or more positions; and

(j) a fragment of the polypeptide of (f), (g), (h), or (i).

In further embodiments the process according to the invention comprises contacting said phospholipid or lysophospholipid with a polypeptide, which has phospholipase C activity, and is selected from the group consisting of:

(a) a polypeptide having at least 60% sequence identity to the mature polypeptide of SEQ ID NO: 2,

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, (ii) the genomic DNA sequence comprising the mature polypeptide coding sequence of any one of SEQ ID NO: 1, or (iii) the full-length complementary strand of (i) or (ii);

(c) a polypeptide encoded by a polynucleotide having at least 60% sequence identity to the mature polypeptide coding sequence of SEQ ID NO: 1;

(d) a variant of the mature polypeptide of SEQ ID NO: 2, comprising a substitution, deletion, and/or insertion at one or more positions; and

(e) a fragment of the polypeptide of (a), (b), (c), or (d);

and

with a polypeptide, which has phospholipase C activity, is derived from a bacterium and is selected from the group consisting of:

(f) a polypeptide having at least 60% sequence identity to the mature polypeptide of SEQ ID NO: 39;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 38 (ii) the genomic DNA sequence comprising the mature polypeptide coding sequence of SEQ ID NO: 38, or (iii) the full-length complementary strand of (i) or (ii);

(h) a polypeptide encoded by a polynucleotide having at least 60% sequence identity to the mature polypeptide coding sequence of SEQ ID NO: 38;

(i) a variant of the mature polypeptide of SEQ ID NO: 39, comprising a substitution, deletion, and/or insertion at one or more positions; and

(j) a fragment of the polypeptide of (f), (g), (h), or (i).

In other embodiments the process according to the invention comprises contacting said phospholipid or lysophospholipid with a polypeptide, which has phospholipase C activity, and is selected from the group consisting of:

(a) a polypeptide having at least 60% sequence identity to the mature polypeptide of SEQ ID NO: 4,

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 3, (ii) the genomic DNA sequence comprising the mature polypeptide coding sequence of any one of SEQ ID NO: 3, or (iii) the full-length complementary strand of (i) or (ii);

(c) a polypeptide encoded by a polynucleotide having at least 60% sequence identity to the mature polypeptide coding sequence of SEQ ID NO: 3;

(d) a variant of the mature polypeptide of SEQ ID NO: 4, comprising a substitution, deletion, and/or insertion at one or more positions; and

(e) a fragment of the polypeptide of (a), (b), (c), or (d);

and

with a polypeptide, which has phospholipase C activity, is derived from a bacterium and is selected from the group consisting of:

(f) a polypeptide having at least 60% sequence identity to the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44 (ii) the genomic DNA sequence comprising the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44, or (iii) the full-length complementary strand of (i) or (ii);

(h) a polypeptide encoded by a polynucleotide having at least 60% sequence identity to the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44;

(i) a variant of the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45, comprising a substitution, deletion, and/or insertion at one or more positions; and

(j) a fragment of the polypeptide of (f), (g), (h), or (i).

In other embodiments the process according to the invention comprises contacting said phospholipid or lysophospholipid with a polypeptide, which has phospholipase C activity, and is selected from the group consisting of:

(a) a polypeptide having at least 60% sequence identity to the mature polypeptide of SEQ ID NO: 4,

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 3, (ii) the genomic DNA sequence comprising the mature polypeptide coding sequence of any one of SEQ ID NO: 3, or (iii) the full-length complementary strand of (i) or (ii);

(c) a polypeptide encoded by a polynucleotide having at least 60% sequence identity to the mature polypeptide coding sequence of SEQ ID NO: 3;

(d) a variant of the mature polypeptide of SEQ ID NO: 4, comprising a substitution, deletion, and/or insertion at one or more positions; and

(e) a fragment of the polypeptide of (a), (b), (c), or (d);

and

with a polypeptide, which has phospholipase C activity, is derived from a bacterium and is selected from the group consisting of:

(f) a polypeptide having at least 60% sequence identity to the mature polypeptide of SEQ ID NO: 39;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 38 (ii) the genomic DNA sequence comprising the mature polypeptide coding sequence of SEQ ID NO: 38, or (iii) the full-length complementary strand of (i) or (ii);

(h) a polypeptide encoded by a polynucleotide having at least 60% sequence identity to the mature polypeptide coding sequence of SEQ ID NO: 38;

(i) a variant of the mature polypeptide of SEQ ID NO: 39, comprising a substitution, deletion, and/or insertion at one or more positions; and

(j) a fragment of the polypeptide of (f), (g), (h), or (i).

In still other embodiments, the process according to the invention comprises contacting said phospholipid or lysophospholipid with a polypeptide, which has phospholipase C activity and is selected from the group consisting of:

(a) a polypeptide having at least 60% sequence identity to the mature polypeptide of SEQ ID NO: 7;

(b) a polypeptide encoded by a polynucleotide that hybridizes very low stringency conditions, low stringency conditions, medium stringency conditions, medium-high stringency conditions, high stringency conditions, or very high stringency conditions with (i) the mature polypeptide coding sequence of SEQ ID NO: 5 (ii) the genomic DNA sequence comprising the mature polypeptide coding sequence of SEQ ID NO: 5, (iii) the cDNA sequence of SEQ ID NO: 5 (SEQ ID NO: 6) or (iv) the full-length complementary strand of (i), (ii) or (iii);

(c) a polypeptide encoded by a polynucleotide having at least 60% sequence identity to the mature polypeptide coding sequence of anyone of SEQ ID NO: 5;

(d) a variant of the mature polypeptide of SEQ ID NO: 7 comprising a substitution, deletion, and/or insertion at one or more positions; and

(e) a fragment of the polypeptide of (a), (b), (c), or (d);

and

with a polypeptide, which has phospholipase C activity and is selected from the group consisting of:

(f) a polypeptide having at least 60% sequence identity to the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44 (ii) the genomic DNA sequence comprising the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44, or (iii) the full-length complementary strand of (i) or (ii);

(h) a polypeptide encoded by a polynucleotide having at least 60% sequence identity to the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44;

(i) a variant of the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45, comprising a substitution, deletion, and/or insertion at one or more positions; and

(j) a fragment of the polypeptide of (f), (g), (h), or (i).

In other embodiments the process according to the invention comprises contacting said phospholipid or lysophospholipid with a polypeptide, which has phospholipase C activity and is selected from the group consisting of:

(a) a polypeptide having at least 60% sequence identity to the mature polypeptide of SEQ ID NO: 7;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 5 (ii) the genomic DNA sequence comprising the mature polypeptide coding sequence of SEQ ID NO: 5, (iii) the cDNA sequence of SEQ ID NO: 5 (SEQ ID NO: 6) or (iv) the full-length complementary strand of (i), (ii) or (iii);

(c) a polypeptide encoded by a polynucleotide having at least 60% sequence identity to the mature polypeptide coding sequence of anyone of SEQ ID NO: 5;

(d) a variant of the mature polypeptide of SEQ ID NO: 7 comprising a substitution, deletion, and/or insertion at one or more positions; and

(e) a fragment of the polypeptide of (a), (b), (c), or (d);

and

with a polypeptide, which has phospholipase C activity, is derived from a bacterium and is selected from the group consisting of:

(f) a polypeptide having at least 60% sequence identity to the mature polypeptide of SEQ ID NO: 39;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 38 (ii) the genomic DNA sequence comprising the mature polypeptide coding sequence of SEQ ID NO: 38, or (iii) the full-length complementary strand of (i) or (ii);

(h) a polypeptide encoded by a polynucleotide having at least 60% sequence identity to the mature polypeptide coding sequence of SEQ ID NO: 38;

(i) a variant of the mature polypeptide of SEQ ID NO: 39, comprising a substitution, deletion, and/or insertion at one or more positions; and

(j) a fragment of the polypeptide of (f), (g), (h), or (i).

In even further embodiments the process according to the invention comprises contacting said phospholipid or lysophospholipid with a polypeptide, which has phospholipase C activity and is selected from the group consisting of:

(a) a polypeptide having at least 60% sequence identity to the mature polypeptide of SEQ ID NO: 7;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 5 (ii) the genomic DNA sequence comprising the mature polypeptide coding sequence of SEQ ID NO: 5, (iii) the cDNA sequence of SEQ ID NO: 5 (SEQ ID NO: 6) or (iv) the full-length complementary strand of (i), (ii) or (iii);

(c) a polypeptide encoded by a polynucleotide having at least 60% sequence identity to the mature polypeptide coding sequence of SEQ ID NO: 5;

(d) a variant of the mature polypeptide of SEQ ID NO: 7 comprising a substitution, deletion, and/or insertion at one or more positions; and

(e) a fragment of the polypeptide of (a), (b), (c), or (d);

and

with a polypeptide, which has phospholipase C activity and is selected from the group consisting of:

(f) a polypeptide having at least 60% sequence identity to the mature polypeptide of any one of SEQ ID NO: 9, SEQ ID NO: 12, SEQ ID NO: 15, SEQ ID NO: 18, SEQ ID NO: 21 SEQ ID NO: 24 and SEQ ID NO: 43;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42, (ii) the genomic DNA sequence comprising the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42, or (iii) the full-length complementary strand of (i) or (ii);

(h) a polypeptide encoded by a polynucleotide having at least 60% sequence identity to the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 44;

(i) a variant of the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 45, comprising a substitution, deletion, and/or insertion at one or more positions; and

(j) a fragment of the polypeptide of (f), (g), (h), or (i);

and

with a polypeptide, which has phospholipase C activity and is selected from the group consisting of:

(k) a polypeptide having at least 60% sequence identity to the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45;

(l) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44 (ii) the genomic DNA sequence comprising the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44, or (iii) the full-length complementary strand of (i) or (ii);

(m) a polypeptide encoded by a polynucleotide having at least 60% sequence identity to the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44;

(n) a variant of the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45, comprising a substitution, deletion, and/or insertion at one or more positions; and

(o) a fragment of the polypeptide of (k), (l), (m), or (n).

In still other embodiments the process according to the invention comprises contacting said phospholipid or lysophospholipid with a polypeptide, which has phospholipase C activity and is selected from the group consisting of:

(a) a polypeptide having at least 60% sequence identity to the mature polypeptide of SEQ ID NO: 7;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 5 (ii) the genomic DNA sequence comprising the mature polypeptide coding sequence of SEQ ID NO: 5, (iii) the cDNA sequence of SEQ ID NO: 5 (SEQ ID NO: 6) or (iv) the full-length complementary strand of (i), (ii) or (iii);

(c) a polypeptide encoded by a polynucleotide having at least 60% sequence identity to the mature polypeptide coding sequence of anyone of SEQ ID NO: 5;

(d) a variant of the mature polypeptide of SEQ ID NO: 7 comprising a substitution, deletion, and/or insertion at one or more positions; and

(e) a fragment of the polypeptide of (a), (b), (c), or (d);

and

a polypeptide, which has phospholipase C activity, is derived from a bacterium and is selected from the group consisting of:

(f) a polypeptide having at least 60% sequence identity to the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 43;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42, (ii) the genomic DNA sequence comprising the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42, or (iii) the full-length complementary strand of (i) or (ii);

(h) a polypeptide encoded by a polynucleotide having at least 60% sequence identity to the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42;

(i) a variant of the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 45, comprising a substitution, deletion, and/or insertion at one or more positions; and

(j) a fragment of the polypeptide of (f), (g), (h), or (i);

and

a polypeptide, which has phospholipase C activity, is derived from a bacterium and is selected from the group consisting of:

(k) a polypeptide having at least 60% sequence identity to the mature polypeptide of SEQ ID NO: 39;

(l) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 38 (ii) the genomic DNA sequence comprising the mature polypeptide coding sequence of SEQ ID NO: 38, or (iii) the full-length complementary strand of (i) or (ii);

(m) a polypeptide encoded by a polynucleotide having at least 60% sequence identity to the mature polypeptide coding sequence of SEQ ID NO: 40;

(n) a variant of the mature polypeptide of SEQ ID NO: 39, comprising a substitution, deletion, and/or insertion at one or more positions; and

(o) a fragment of the polypeptide of (k), (l), (m), or (n).

According to some embodiments the invention also provides a process comprising contacting said phospholipid or lysophospholipid with a polypeptide, which has phospholipase C activity and is selected from the group consisting of:

(a) a polypeptide having at least 60% sequence identity to the mature polypeptide of any one of SEQ ID NO: 2,

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, (ii) the genomic DNA sequence comprising the mature polypeptide coding sequence of any one of SEQ ID NO: 1, or (iii) the full-length complementary strand of (i) or (ii);

(c) a polypeptide encoded by a polynucleotide having at least 60% sequence identity to the mature polypeptide coding sequence of SEQ ID NO: 1;

(d) a variant of the mature polypeptide of SEQ ID NO: 2, comprising a substitution, deletion, and/or insertion at one or more positions; and

(e) a fragment of the polypeptide of (a), (b), (c), or (d);

and

with a polypeptide, which has phosplipase C activity and is derived from a bacterium, said polypeptide being selected from the group consisting of:

(f) a polypeptide having at least 60% sequence identity to the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 43;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42, (ii) the genomic DNA sequence comprising the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42, or (iii) the full-length complementary strand of (i) or (ii);

(h) a polypeptide encoded by a polynucleotide having at least 60% sequence identity to the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42;

(i) a variant of the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 43, comprising a substitution, deletion, and/or insertion at one or more positions; and

(j) a fragment of the polypeptide of (f), (g), (h), or (i);

and

with a polypeptide, which has phospholipase C activity and is derived from a bacterium is selected from the group consisting of:

(k) a polypeptide having at least 60% sequence identity to the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45;

(l) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44 (ii) the genomic DNA sequence comprising the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44, or (iii) the full-length complementary strand of (i) or (ii);

(m) a polypeptide encoded by a polynucleotide having at least 60% sequence identity to the mature polypeptide coding sequence of any one of SEQ ID NO: 25, SEQ ID NO: 28, SEQ ID NO: 30, SEQ ID NO: 33, SEQ ID NO: 35, SEQ ID NO: 38 and SEQ ID NO: 44;

(n) a variant of the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45, comprising a substitution, deletion, and/or insertion at one or more positions; and

(o) a fragment of the polypeptide of (k), (l), (m), or (n).

In even further embodiments the invention provides a process comprising contacting said phospholipid or lysophospholipid with a polypeptide, which has phospholipase C activity and is selected from the group consisting of

(a) a polypeptide having at least 60% sequence identity to the mature polypeptide of any one of SEQ ID NO: 2,

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, (ii) the genomic DNA sequence comprising the mature polypeptide coding sequence of any one of SEQ ID NO: 1, or (iii) the full-length complementary strand of (i) or (ii);

(c) a polypeptide encoded by a polynucleotide having at least 60% sequence identity to the mature polypeptide coding sequence of SEQ ID NO: 1;

(d) a variant of the mature polypeptide of SEQ ID NO: 2, comprising a substitution, deletion, and/or insertion at one or more positions; and

(e) a fragment of the polypeptide of (a), (b), (c), or (d);

and

a polypeptide, which has phospholipase C activity, is derived from a bacterium, and is selected from the group consisting of:

(f) a polypeptide having at least 60% sequence identity to the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 43;

(g) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42, (ii) the genomic DNA sequence comprising the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42, or (iii) the full-length complementary strand of (i) or (ii);

(h) a polypeptide encoded by a polynucleotide having at least 60% sequence identity to the mature polypeptide coding sequence of any one of SEQ ID NO: 8, SEQ ID NO: 11, SEQ ID NO: 14, SEQ ID NO: 17, SEQ ID NO: 20, SEQ ID NO: 23 and SEQ ID NO: 42;

(i) a variant of the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 43, comprising a substitution, deletion, and/or insertion at one or more positions; and

(j) a fragment of the polypeptide of (f), (g), (h), or (i);

and

a polypeptide, which has phospholipase C activity, is derived from a bacterium is selected from the group consisting of:

(k) a polypeptide having at least 60% sequence identity to the mature polypeptide of SEQ ID NO: 39;

(l) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 38 (ii) the genomic DNA sequence comprising the mature polypeptide coding sequence of SEQ ID NO: 38, or (iii) the full-length complementary strand of (i) or (ii);

(m) a polypeptide encoded by a polynucleotide having at least 60% sequence identity to the mature polypeptide coding sequence of SEQ ID NO: 38;

(n) a variant of the mature polypeptide of SEQ ID NO: 39, comprising a substitution, deletion, and/or insertion at one or more positions; and

(o) a fragment of the polypeptide of (k), (l), (m), or (n).

A further aspect of the invention provides a process for hydrolysing a phospholipid or lysophospholipid, comprising contacting said phospholipid or lysophospholipid with at least one polypeptide, which has phospholipase C activity and is derived from a fungus; and at least one polypeptide, which has phospholipase C activity, and is selected from the group consisting of:

(a) a polypeptide having at least 60% sequence identity to the mature polypeptide of any one of SEQ ID NO: 41;

(b) a polypeptide encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 40 (ii) the genomic DNA sequence comprising the mature polypeptide coding sequence of any one of SEQ ID NO: 40, (iii) the cDNA sequence of SEQ ID NO: 40 or (iv) the full-length complementary strand of (i), (ii) or (iii);

(c) a polypeptide encoded by a polynucleotide having at least 60% sequence identity to the mature polypeptide coding sequence of SEQ ID NO: 40;

(d) a variant of the mature polypeptide of SEQ ID NO: 41 comprising a substitution, deletion, and/or insertion at one or more positions; and

(e) a fragment of the polypeptide of (a), (b), (c), or (d).

(f) PURIFINE® PLC, which is commercially available from DSM

As mentioned above, variants of the mature polypeptide of SEQ ID NO 41 and of PURIFINE® PLC are disclosed in international patent publication WO 2011/0146812. In particular, the variants of SEQ ID NO 41 may have one or more of the amino acid changes or substitutions described in Table 12 (page 210), Table 13 (pages 211-18), Table 14 (pages 220-21) and/or table 15 (pages 222-23) in the said WO-publication. The content of all four tables is incorporated herein by reference in its entirety.

In particular embodiments of the process, the said mature polypeptide comprises or consists of amino acid residues 24-328 of SEQ ID NO: 41 and/or the said variant in (d) comprises amino acid residues 56-195 of SEQ IN NO: 41 and/or the said fragment in (e) comprises amino acid residues 56-195 of SEQ ID NO: 41.

Also, in the embodiments of the invention provided above, the polypeptide having at least 60% sequence identity to the mature polypeptide of any one of SEQ ID NO: 9, SEQ ID NO: 12, SEQ ID NO: 15, SEQ ID NO: 18, SEQ ID NO: 21, SEQ ID NO: 24 and SEQ ID NO: 43 preferably has a length of 280-320 amino acid residues, such as a length of 280-310 amino acid residues, 280-305 amino acid residues, 280-300 amino acid residues, 280-298 amino acid residues 280-297 amino acid residues, 280-296 amino acid residues, 285-320 amino acid residues, 285-315 amino acid residues, 285-310 amino acid residues, 285-305 amino acid residues, 285-300 amino acid residues, 285-298 amino acid residues, 285-297 amino acid residues, 285-296 amino acid residues, 290-320 amino acid residues, 290-315 amino acid residues, 290-310 amino acid residues, 290-305 amino acid residues, 290-300 amino acid residues, 290-298 amino acid residues, 290-297 amino acid residues, 290-296 amino acid residues, 295-320 amino acid residues, 295-315 amino acid residues, 295-310 amino acid residues, 295-305 amino acid residues, 295-300 amino acid residues, 295-298 amino acid residues, 255-297 amino acid residues, or a length of 295-296 amino acid residues.

Similarly, the polypeptide having at least 60% sequence identity to the mature polypeptide of any one of SEQ ID NO: 26, SEQ ID NO: 29, SEQ ID NO: 31, SEQ ID NO: 34, SEQ ID NO: 36, SEQ ID NO: 39 and SEQ ID NO: 45 preferably has a length of 220-280 amino acid residues, such as a length of 220-270 amino acid residues, 220-260 amino acid residues, 220-250 amino acid residues, 220-248 amino acid residues 220-246 amino acid residues, 220-244 amino acid residues, 225-280 amino acid residues, 225-270 amino acid residues, 225-260 amino acid residues, 225-250 amino acid residues, 225-248 amino acid residues 225-246 amino acid residues, 225-244 amino acid residues, 230-280 amino acid residues, 230-270 amino acid residues, 230-260 amino acid residues, 230-250 amino acid residues, 230-248 amino acid residues, 230-246 amino acid residues, 230-244 amino acid residues, 235-280 amino acid residues, 235-270 amino acid residues, 235-260 amino acid residues, 235-250 amino acid residues, 235-248 amino acid residues 235-246 amino acid residues, 235-244 amino acid residues, 240-280 amino acid residues, 240-270 amino acid residues, 240-260 amino acid residues, 240-250 amino acid residues, 240-248 amino acid residues 240-246 amino acid residues, 240-244 amino acid residues, 242-280 amino acid residues, 242-270 amino acid residues, 242-260 amino acid residues, 242-250 amino acid residues, 242-248 amino acid residues 242-246 amino acid residues, 242-244 amino acid residues, 243-280 amino acid residues, 243-270 amino acid residues, 243-260 amino acid residues, 243-250 amino acid residues, 243-248 amino acid residues 243-246 amino acid residues, 243-244 amino acid residues,

It will be understood that the process according to this aspect of the invention may have any of the characteristics and features set forth in relation to the processes of the other aspects of the invention provided above. Likewise, it will be understood that the plurality of polypeptides having Phospholipase C activity used in the process according to this aspect of the invention may be as described for the pluralities of polypeptides having Phospholipase C activity the processes of the other aspects of the invention provided above. As the skilled person will also realize the process of the invention may comprise contacting a phospholipid or lysophospholipid with at least one of the polypeptides which has phospholipase C activity and is derived from a fungus; and at least one polypeptide which has phospholipase C activity and is derived from a bacterium, either simultaneous or in sequence.

When the contacting is done simultaneously, the polypeptides may be in a composition as described above, or may be added individually at the same time. When the polypeptides are added in sequence, the at least one of the polypeptides which has phospholipase C activity and is derived from a fungus may be added first and the addition of said at least one polypeptide which has phospholipase C activity and is derived from a bacterium be delayed, such as by 5 minutes-3 hours, 10 minutes-3 hours, 20 minutes-3 hours, 30 minutes-3 hours, 1-3 hours, 2-3 hours, 5 minutes-2 hours, 10 minutes-2 hours, 20 minutes-2 hours, 30 minutes-2 hours, 1-2 hours, 5 minutes-1.5 hours, 10 minutes-1.5 hours, 20 minutes-1.5 hours, 30 minutes-1.5 hours, 1-1.5 hours, 5 minutes-1 hour, 10 minutes-1 hour, 20 minutes-1 hour, 30 minutes-1 hour, 5-30 minutes, 10-30 minutes or 20-30 minutes.

Alternatively the at least one of the polypeptides which has phospholipase C activity and is derived from a bacterium may be added first and the addition of said at least one polypeptide which has phospholipase C activity and is derived from a fungus be delayed, such as by 5 minutes-3 hours, 10 minutes-3 hours, 20 minutes-3 hours, 30 minutes-3 hours, 1-3 hours, 2-3 hours, 5 minutes-2 hours, 10 minutes-2 hours, 20 minutes-2 hours, 30 minutes-2 hours, 1-2 hours, 5 minutes-1.5 hours, 10 minutes-1.5 hours, 20 minutes-1.5 hours, 30 minutes-1.5 hours, 1-1.5 hours, 5 minutes-1 hour, 10 minutes-1 hour, 20 minutes-1 hour, 30 minutes-1 hour, 5-30 minutes, 10-30 minutes or 20-30 minutes.

In further embodiments of the invention the composition comprises one or more enzyme activities in addition to the said plurality of polypeptides having phospholipase C activity. In particular, the composition may comprise one or more polypeptides having phopolipase A activity. The one or more polypeptides having phospholipase A activity may in particular be an isolated polypeptide having phospholipase A activity, selected from the group consisting of:

(a) a polypeptide having at least 80% sequence identity, such as at least 85%, at least 90%, at least 95% or at least 98% sequence identity, to the mature polypeptide of SEQ ID NO: 46 or to the polypeptide of SEQ ID NO: 47;

(b) a fragment of the polypeptide of (a) that has phospholipase A activity.

The said phospholipid may be in a composition comprising triglycerides. The composition may be an oil, such as a vegetable oil; e.g. an edible vegetable oil. The process may further comprise contacting said phospholipid or lysophospholipid or said composition comprising triglycerides, such as said oil, with the plurality of polypeptides having Phospholipase C activity under conditions sufficient for said polypeptides having phospholipase C activity to react with said phospholipid or lysophospholipid to create diglyceride and phosphate ester.

Additionally, the process according to the invention may comprise separating the phosphate ester from the triglycerides or the oil.

According to particular embodiments of the invention said phospholipid is in a composition comprising triglycerides. The composition may in particular be an oil, such as a vegetable oil; e.g. an edible vegetable oil.

PI-specific PLC converts phosphatidyl inositol (PI) to diglyceride and phosphoinositol. PC-specific PLC converts phosphatidylcholine (PC) to diglyceride and phosphocholine. PE-specific PLC converts phosphatidylethanolamine (PE) to diglyceride and phophoethanolamine. The diglyceride stays in the oil phase (improving oil yield) and the phosphorous-containing moieties separates into the aqueous phase where it is removed as a component of the heavy phase during centrifugation. The gum phase (heavy phase) may be treated further with a phospholipase or composition of the present invention to increase hydrolysis of phospholipids in the gum fraction from water degumming to release entrapped triglyceride oil. This is particular useful when the degumming process has not already applied phospholipases. Compositions or phospholipases according to the invention can be incorporated into either a water degumming or a chemical or physical oil refining process with preferably less than 10%, 9%, 8%, 7%, 6% or 5% water, even more preferably less than 4%, 3% or 2% water, preferably at 50° C. or above, even more preferably at 60° C. or above. In a preferred embodiment the compositions of the invention are incorporated into a water degumming process, caustic refining process or acid degumming process.

In another preferred embodiment the phospholipases of the invention are incorporated into a physical refining process applying citric acid or phosphoric acid and sodium hydroxide to facilitate hydratability of insoluble phospholipids and ensure an environment suitable for the enzyme with preferably less than 0.15% citric acid or phosphoric acid, even more preferably less than 0.1%, 0.09%, 0.08%, 0.07%, 0.06% or 0.05%; and less than 4%, 3% or 2% water, preferably at 50° C. or above, even more preferably at 60° C. or above.

In other embodiments the degumming process is a caustic refining process or acid degumming process

Phospholipids are commonly measured in oil as “phosphorous content” in parts per million. Table 1 sets forth the typical amounts of phospholipids present in the major oilseed crops, and the distribution of the various functional groups as a percentage of the phospholipids present in the crude oil.

TABLE 1
Typical levels and phospholipid distributions
in the crude oil from common oilseeds

| | Soy Oil | Canola Oil | Sunflower Oil |
|---|---|---|---|
| Phosphorous (ppm) | 400-1500 | 200-900 | 300-700 |
| PC % | 12-46 | 25-40 | 29-52 |
| PE % | 8-34 | 15-25 | 17-26 |
| PA % | 17-26 | 10-20 | 15-30 |
| PI % | 2-15 | 2-25 | 11-22 |

The compositions and processes of the invention can be used to achieve a more complete degumming of high phosphorous oils, e.g. an oil with more than 200 ppm of phosphorous, preferably more than 300 ppm, 400 ppm, 500 ppm, 600 ppm, 700 ppm, 800 ppm, 900 ppm, even more preferred the oil contains more than 1000 ppm phosphorous.

Preferably the oil comprises phosphatidic acid (PA), phosphatidylcholine (PC), phosphatidylethanolamine (PE) and phosphatidyl inositol (PI). Preferably the oil contains more than 50 ppm phosphorous originating from phosphatidyl inositol (PI), more preferably it contains more than 75 ppm, 100 ppm, 125 ppm PI, even more preferably it contains more than 150 ppm, most preferably it contains more than 175 ppm phosphorous originating from PI. Preferably the oil contains more than 100 ppm phosphorous originating from phosphatidylcholine (PC), more preferably it contains more than 150 ppm, 200 ppm, 250 ppm PC, even more preferably it contains more than 300 ppm, most preferably it contains more than 400 ppm phosphorous originating from PC. Preferably the oil contains more than 75 ppm phosphorus originating from phosphatidylethanolamine (PE), more preferably it contains more than 100 ppm, 125 ppm, 150 ppm PE, even more preferably it contains more than 200 ppm, most preferably it contains more than 300 ppm phosphorous originating from PE. The oil may also contain more than 5 ppm phosphorus originating from phosphatidic acid (PA), such as more than 10 ppm phosphorus originating from phosphatidic acid (PA), more than 15 ppm, 20 ppm, 25 ppm, 40 ppm, 50 ppm PE, or 75 ppm phosphorous originating from PA.

In a preferred embodiment the oil is an edible oil. More preferred, the edible oil is selected from rice bran, rapeseeds, palm, peanuts and other nuts, soybean, corn, canola, and sunflower oils. The plurality of polypeptides having Phospholipase C activity and the compositions of the invention can be used in any “degumming” procedure, including water degumming, ALCON oil degumming (e.g., for soybeans), safinco degumming, “super degumming,” UF degumming, TOP degumming, uni-degumming, dry degumming and ENZYMAX™ degumming. See, for example, WO 2007/103005, US 2008/0182322, U.S. Pat. No. 6,355,693, U.S. Pat. No. 6,162,623, U.S. Pat. No. 6,103,505, U.S. Pat. No. 6,001,640, U.S. Pat. No. 5,558,781 and U.S. Pat. No. 5,264,367 for description of degumming processes where phospholipases of the present invention can be applied. Various “degumming” procedures incorporated by the methods of the invention are described in Bockisch, M. (1998) In Fats and Oils Handbook, The extraction of Vegetable Oils (Chapter 5), 345-5 445, AOCS Press, Champaign, Ill. The plurality of polypeptides having Phospholipase C activity and compositions of the invention can be used in the industrial application of enzymatic degumming of triglyceride oils as described, e.g., in EP 513 709. In a further embodiment the oil is selected from crude oil, water degummed oil, caustic refined oil and acid degummed oil. The water-degumming of a crude oil or fat may be achieved by thoroughly mixing hot water and warm oil or fat having a temperature of between 50° C. to 90° C. for 30 to 60 minutes. This process serves to partially remove the hydratable phospholipids. Also, an acid treatment may be performed before the enzymatic degumming, where the acid used is selected from the group consisting of phosphoric acid, acetic acid, citric acid, tartaric acid, succinic acid, and mixtures thereof, in particular a treatment using citric acid or phosphoric acid are preferred. The acid treatment is preferably followed by a neutralization step to adjust the pH between about 4.0 to 7.0, more preferably from 4.5 to 6.5, preferably using NaOH or KOH. The acid treatment serves to chelate metals bound to the phospholipids hereby making a more hydratable form. Preferably, the phospholipases as described herein is added after water degumming or acid treatment of the oil. It is also possible to perform the degumming step using the plurality of polypeptides having Phospholipase C activity and/or the compositions as described herein on a crude oil or fat, i.e. an oil or fat not previously water degummed or acid treated.

In one aspect, the invention provides methods for enzymatic degumming under conditions of low water, e.g., in the range of between about 0.1% to 20% water or 0.5% to 10% water. In one aspect, this results in the improved separation of a heavy phase from the oil phase during centrifugation. The improved separation of these phases can result in more efficient removal of phospholipids from the oil, including both hydratable and nonhydratable phospholipids. In one aspect, this can produce a gum fraction that contains less entrained neutral oil (triglycerides), thereby improving the overall yield of oil during the degumming process. In one aspect, the plurality of polypeptides having Phospholipase C activity and the compositions of the invention are used to treat oils to reduce gum mass and increase neutral oil gain through reduced oil entrapment. In one aspect, the plurality of polypeptides having Phospholipase C activity and the compositions of the invention are used for diacylglycerol (DAG) production and to contribute to the oil phase.

In particular embodiments, the oil is contacted with 0.5-200 mg enzyme protein (EP)/Kg oil of said fungal and bacterial phospholipases; such as with 0.5-100 mg enzyme protein (EP)/Kg oil of said fungal and bacterial phospholipases, with 0.5-25 mg enzyme protein (EP)/Kg oil of said fungal and bacterial phospholipases, with 0.5-15 mg enzyme protein (EP)/Kg oil of said fungal and bacterial phospholipase. with 0.5-10 mg enzyme protein (EP)/Kg oil of said fungal and bacterial phospholipase, with 0.5-5 mg enzyme protein (EP)/Kg oil of said fungal and bacterial phospholipase, with 1-200 mg enzyme protein (EP)/Kg oil of said fungal and bacterial phospholipase, with 1-100 mg enzyme protein (EP)/Kg oil of said fungal and bacterial phospholipase, with 1-25 mg enzyme protein (EP)/Kg oil of said fungal and bacterial phospholipase, with 1-15 mg enzyme protein (EP)/Kg oil of said fungal and bacterial phospholipase, with 1-10 mg enzyme protein (EP)/Kg oil of said fungal and bacterial phospholipase, with 1-5 mg enzyme protein (EP)/Kg oil of said fungal and bacterial phospholipase, with 2-200 mg enzyme protein (EP)/Kg oil of said fungal and bacterial phospholipase, with 2-100 mg enzyme protein (EP)/Kg oil of said fungal and bacterial phospholipase, with 2-50 mg enzyme protein (EP)/Kg oil of said fungal and bacterial phospholipase, with 2-25 mg enzyme protein (EP)/Kg oil of said fungal and bacterial phospholipase, with 2-15 mg enzyme protein (EP)/Kg oil of said fungal and bacterial phospholipase, with 2-10 mg enzyme protein (EP)/Kg oil of said fungal and bacterial phospholipase, with 2-7 mg enzyme protein (EP)/Kg oil of said fungal and bacterial phospholipase, or with 2-5 mg enzyme protein (EP)/Kg oil of said fungal and bacterial phospholipase.

In preferred embodiments the oil is contacted with 5-50 mg enzyme protein (EP)/Kg oil of said fungal phospholipase; and 0.5-20 mg enzyme protein (EP)/Kg oil of said bacterial phospholipase. In further embodiments, the oil is contacted with 5-40 mg enzyme protein (EP)/Kg oil of said fungal phospholipase; and 1-15 mg enzyme protein (EP)/Kg oil of said bacterial phospholipase. In still further embodiments, the oil is contacted with 5-30 mg enzyme protein (EP)/Kg oil of said fungal phospholipase; and 1-15 mg enzyme protein (EP)/Kg oil of said bacterial phospholipase. In other embodiments, the oil is contacted with 10-30 mg enzyme protein (EP)/Kg oil of said fungal phospholipase; and 1-5 mg enzyme protein (EP)/Kg oil of said bacterial phospholipase.

The phospholipase treatment can be conducted by dispersing an aqueous solution of the phospholipase, preferably as droplets with an average diameter below 10 microM. The amount of water is preferably 0.5-5% by weight in relation to the oil. An emulsifier may optionally be added. Mechanical agitation may be applied to maintain the emulsion. Agitation may be done with a high shear mixer with a tip speed above 1400 cm/s.

In certain embodiments, a suitable oil degumming method comprises a) mixing an aqueous acid with an oil to obtain an acidic mixture having pH of about 1 to 4, b) mixing a base with the acidic mixture to obtain a reacted mixture having pH of about 6-9, and c) degumming the reacted mixture with a plurality of polypeptides having Phospholipase C activity and/or a composition of the present invention to obtain a degummed oil. In certain embodiments, mixing in steps a) and/or b) creates an emulsion that comprises an aqueous phase in average droplet size between about 15 microM to about 45 microM. In certain embodiments, mixing in steps a) and/or b) creates an emulsion that comprises at least about 60% of an aqueous phase by volume in droplet size between about 15 microM to about 45 microM in size, wherein percentage of the aqueous phase is based on the total volume of the aqueous phase. Any acid deemed suitable by one of skill in the art can be used in the methods provided herein. In certain embodiments, the acid is selected from the group consisting of phosphoric acid, acetic acid, citric acid, tartaric acid, succinic acid, and a mixture thereof. Any acid deemed suitable by one of skill in the art can be used in the methods provided herein. In certain embodiments, the base is selected from the group consisting of sodium hydroxide, potassium hydroxide, sodium silicate, sodium carbonate, calcium carbonate, and a combination thereof.

In a preferred embodiment the phospholipase treatment can be conducted at a pH in the range of about 4.0 to 7.0, more preferably from 4.5 to 6.5. The pH is measured in the emulsion or in the interphase between the oil and aqueous solution. A suitable temperature is generally 30-80° C. In a preferred embodiment the temperature of the oil is between 50 and 70° C., more preferred between 55 and 65° C. and most preferred between 50 and 60° C. In other preferred embodiments the temperature of the oil is between 60 and 80° C., more preferred between 65 and 75° C. and most preferred between 67 and 72° C.

The reaction time will typically be 1-12 hours (e.g., 1-6 hours, or 1-3 hours, most preferred the reaction time is between 1.5 and 4 hours, even more preferred between 1.5 and 2 hours). A suitable enzyme dosage will usually be 0.1-10 mg per liter (e.g., 0.5-5 mg per liter). The phospholipase treatment may be conducted batch wise, e.g., in a tank with stirring, or it may be continuous, e.g., a series of stirred tank reactors. The phospholipase treatment may be followed by separation of an aqueous phase and an oil phase. The separation may be performed by conventional means, e.g., centrifugation. When a liquid lipase is used, the aqueous phase will contain phospholipase, and the enzyme may be re-used to improve the process economy.

In a preferred embodiment of the present invention the treatment reduces the total phosphorous content of the oil to 200 ppm, preferably below 100 ppm, below 50 ppm, preferably below 40 ppm, 30 ppm, 20 ppm, 15 ppm, more preferably below 10 ppm, below 9 ppm, below 8 ppm, below 7 ppm, below 6 ppm, most preferably below 5 ppm.

The compositions and processes of the invention may be used for partial hydrolysis of phospholipids, preferably lecithin, to obtain improved phospholipid emulsifiers. This application is further described in Ullmann's Encyclopedia of Industrial Chemistry (Publisher: VCH Weinheim (1996)), JP patent 2794574, and JP-B 6-087751.

The compositions and processes of the invention can be used to improve the filterability of an aqueous solution or slurry of carbohydrate origin by treating it with the phospholipase. This is particularly applicable to a solution of slurry containing a starch hydrolyzate, especially a wheat starch hydrolyzate, since this tends to be difficult to filter and to give cloudy filtrates. The treatment can be done in analogy with EP 219,269 (CPC International).

The compositions and processes of the invention may be used in a process for the production of an animal feed which comprises mixing the phospholipase with feed substances comprising at least one phospholipid. This can be done in analogy with EP 743 017.

The plurality of polypeptides having Phospholipase C activity and the compositions of the present invention may be used in combination with one or more lipolytic enzymes to convert fats and oils to fatty acid alkyl esters while achieving degumming in the same process. Such a process is for example described in U.S. Pat. No. 8,012,724.

The plurality of polypeptides having Phospholipase C activity and the compositions the invention may be added to and thus be used as a component of a detergent composition. The detergent composition may for example be formulated as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations.

The plurality of polypeptides having Phospholipase C activity and the compositions of the invention may be used for production of dough and baked products from dough, as well as for production of baking compositions and baking additives. The dough generally comprises wheat meal or wheat flour and/or other types of meal, flour or starch such as corn flour, corn starch, rye meal, rye flour, oat flour, oat meal, soy flour, sorghum meal, sorghum flour, potato meal, potato flour or potato starch. The dough may be fresh, frozen or par-baked. The dough is normally leavened dough or dough to be subjected to leavening. The dough may be leavened in various ways, such as by adding chemical leavening agents, e.g., sodium bicarbonate or by adding a leaven (fermenting dough), but it is preferred to leaven the dough by adding a suitable yeast culture, such as a culture of Saccharomyces cerevisiae (baker's yeast), e.g. a commercially available strain of S. cerevisiae. The dough may also comprise other conventional dough ingredients, e.g.: proteins, such as milk powder, gluten, and soy; eggs (either whole eggs, egg yolks or egg whites); an oxidant such as ascorbic acid, potassium bromate, potassium iodate, azodicarbonamide (ADA) or ammonium persulfate; an amino acid such as L-cysteine; a sugar; a salt such as sodium chloride, calcium acetate, sodium sulfate or calcium sulfate. The dough may comprise fat (triglyceride) such as granulated fat or shortening, but the invention is particularly applicable to a dough where less than 1% by weight of fat is added, and particularly to a dough which is made without addition of fat. The dough may further comprise an emulsifier such as mono- or diglycerides, diacetyl tartaric acid esters of mono- or diglycerides, sugar esters of fatty acids, polyglycerol esters of fatty acids, lactic acid esters of monoglycerides, acetic acid esters of monoglycerides, polyoxyethylene stearates, or lysolecithin. The dough may be used for any kind of baked product prepared from dough, either of a soft or a crisp character, either of a white, light or dark type. Examples are bread (in particular white, whole-meal or rye bread), typically in the form of loaves or rolls, French baguette-type bread, pita bread, tortillas, cakes, pancakes, biscuits, wafers, cookies, pie crusts, crisp bread, steamed bread, pizza and the like.

The present invention is further described by the following examples that should not be construed as limiting the scope of the invention.

### EXAMPLES

### Example 1

### P-NMR Assay of Purified PLC Enzymes

### Concept

The assay was conducted by incubating the PLC with a 10:1 mixture of a crude vegetable oil and aqueous citrate buffer, pH 4.0, 5.5 or 7.0. Enzyme concentration was 10 mg/kg and 100 mg/kg (mg enzyme protein (EP) per kg oil). The mixture was incubated with vigorous shaking at 50 C for 2 h. The reaction mixture was then analyzed by P-NMR. This involved an aqueous extraction step during which the phosphor species liberated by the PLC are removed from the oil phase. Hence, only lipophilic P-species were detected, i.e. unreacted phospholipid.

### Assay Procedure

The purified enzyme was diluted to 0.9 mg/mL and 0.09 mg/mL in 100 mM citrate buffer, pH 4.0, 5.5 or 7.0. The assay was initiated by adding 25 uL diluted enzyme to 250 uL crude vegetable oil in a 2 mL Eppendorf tube and incubating the mixture in a thermoshaker at 50 C for 2 h. The oil used was a crude soybean oil containing a significant amount of both PA (128 ppm P), PE (141 ppm P), PI (103 ppm P) and PC (157 ppm P). For assay of mature polypeptide of SEQ ID NO: 4 a fully refined soybean oil spiked with a mixture of PA (180 ppm P), PE (349 ppm P), PI (595 ppm P) and PC (431 ppm P) was used.

### NMR Analysis

To the oil sample was then added 0.500 mL internal standard (IS) solution, followed by 0.5 mL CDCl₃ and 0.5 mL Cs-EDTA buffer. The sample was shaken for 30 s, and then centrifuged (tabletop centrifuge, 3 min, 13,400 rpm) to get phase separation. The lower phase was transferred to a NMR-tube. P-NMR was performed with 128 scans and a delay time of 5 s. Set scale reference according to IS signal (−17.75 ppm). All signals were integrated. Assignments (approx. ppm @ 25 C): 1.7 (PA), −0.1 (PE), −0.5 (PI), −0.8 (PC). The concentration of each species was calculated as “ppm P”, i.e. mg elemental P per kg oil sample. Hence, ppm P=I/I(IS)*n(IS)*M(P)/m(oil). Finally, residual phospholipid content was calculated as the ratio of enzyme treated sample vs. blank.

The internal standard solution is 2 mg/mL triphenylphosphate in methanol.

The Cs-EDTA buffer was prepared as: EDTA (5.85 g) is dispersed in water (approx. 50 mL). The pH was adjusted to 7.5 using 50% w/w CsOH. This gave a clear solution. Water was added up to 100 mL to give a concentration of 0.2 M EDTA.

Results The tables below show residual phospholipid content in percent.
PURIFINE® PLC, 100 mg/kg

| | PA | PE | PI | PC |
|---|---|---|---|---|
| pH 4.0 | 107 | 107 | 91 | 114 |
| pH 5.5 | 107 | 38 | 106 | 20 |
| pH 7.0 | 89 | 0 | 94 | 0 |

10 mg/kg

| | PA | PE | PI | PC |
|---|---|---|---|---|
| pH 4.0 | 106 | 106 | 98 | 105 |
| pH 5.5 | 101 | 114 | 106 | 98 |
| pH 7.0 | 112 | 18 | 102 | 0 |

### Bt-PLC/Mature Polypeptide of SEQ ID NO: 29 (Batch A)

100 mg/kg

| | PA | PE | PI | PC |
|---|---|---|---|---|
| pH 4.0 | 94 | 102 | 91 | 94 |
| pH 5.5 | 92 | 90 | 92 | 70 |
| pH 7.0 | 98 | 19 | 100 | 7 |

10 mg/kg

| | PA | PE | PI | PC |
|---|---|---|---|---|
| pH 4.0 | 100 | 101 | 92 | 92 |
| pH 5.5 | 103 | 100 | 93 | 96 |
| pH 7.0 | 112 | 54 | 103 | 13 |

### Bt-PLC/Mature Polypeptide of SEQ ID NO: 29 (Batch B)

100 mg/kg

| | PA | PE | PI | PC |
|---|---|---|---|---|
| pH 4.0 | n.d. | n.d. | n.d. | n.d. |
| pH 5.5 | n.d. | n.d. | n.d. | n.d. |
| pH 7.0 | 61 | 0 | 90 | 0 |

10 mg/kg

| | PA | PE | PI | PC |
|---|---|---|---|---|
| pH 4.0 | 105 | 103 | 96 | 98 |
| pH 5.5 | 95 | 90 | 99 | 61 |
| pH 7.0 | 103 | 23 | 96 | 6 |

### Mature Polypeptide of SEQ ID NO: 7 (Batch A)

100 mg/kg

| | PA | PE | PI | PC |
|---|---|---|---|---|
| pH 4.0 | 68 | 72 | 77 | 68 |
| pH 5.5 | 26 | 19 | 28 | 33 |
| pH 7.0 | 10 | 19 | 9 | 30 |

10 mg/kg

| | PA | PE | PI | PC |
|---|---|---|---|---|
| pH 4.0 | 90 | 90 | 90 | 91 |
| pH 5.5 | 66 | 77 | 60 | 77 |
| pH 7.0 | n.d. | n.d. | n.d. | n.d. |

### Mature Polypeptide of SEQ ID NO: 7 (Batch B)

100 mg/kg

| | PA | PE | PI | PC |
|---|---|---|---|---|
| pH 4.0 | n.d. | n.d. | n.d. | n.d. |
| pH 5.5 | 17 | 18 | 10 | 16 |
| pH 7.0 | 7 | 0 | 0 | 11 |

10 mg/kg

| | PA | PE | PI | PC |
|---|---|---|---|---|
| pH 4.0 | n.d. | n.d. | n.d. | n.d. |
| pH 5.5 | 45 | 65 | 41 | 29 |
| pH 7.0 | 31 | 65 | 23 | 63 |

### Mature Polypeptide of SEQ ID NO: 2

100 mg/kg

| | PA | PE | PI | PC |
|---|---|---|---|---|
| pH 4.0 | 54 | 54 | 83 | 51 |
| pH 5.5 | 21 | 67 | 51 | 30 |
| pH 7.0 | 23 | 31 | 13 | 17 |

10 mg/kg

| | PA | PE | PI | PC |
|---|---|---|---|---|
| pH 4.0 | 85 | 91 | 90 | 80 |
| pH 5.5 | 63 | 83 | 51 | 61 |
| pH 7.0 | 61 | 78 | 61 | 67 |

### Mature Polypeptide of SEQ ID NO: 4

100 mg/kg

| | PA | PE | PI | PC |
|---|---|---|---|---|
| pH 4.0 | 51 | 49 | 0 | 10 |
| pH 5.5 | 38 | 47 | 0 | 12 |
| pH 7.0 | 0 | 58 | 0 | 16 |

10 mg/kg

| | PA | PE | PI | PC |
|---|---|---|---|---|
| pH 4.0 | 79 | 80 | 43 | 72 |
| pH 5.5 | 94 | 93 | 55 | 64 |
| pH 7.0 | 108 | 82 | 69 | 73 |

### Example 2

### Degumming Assay/Metal Composition of Crude Oil

Performance of the phospholipase C enzyme compositions of the present invention:
Bacterial: a) Bt-PLC (mature polypeptide of SEQ ID NO: 29) and b) Mature polypeptide of SEQ ID NO: 9
Fungal: a) Mature polypeptide of SEQ ID NO:4 and B) Mature polypeptide of SEQ ID NO: 2 and C) Mature polypeptide of SEQ ID NO: 9
PURIFINE® PLC,
   was tested in a degumming assay that mimics industrial scale degumming. The assay measured the following parameters in the oil phase after the degumming:
   a) Diglyceride content by High-performance liquid chromatography (HPLC) coupled to Evaporative Light Scattering Detector (ELSD) or Charged Aerosol Detector (Corona Veo).
   b) Quantification of the individual phospholipids species: Phosphatidylcholine (PC); Phosphatidylinositol (PI); Phosphatidylethanolamine (PE); Phosphatidic acid (PA); by Liquid Chromatography quadrupole mass spectrometer time of flight (LC/TOF/MS)
   c) Total phosphorus reduction by Inductively coupled plasma optical emission spectrometry (ICP-OES).

The phosphorous, calcium, magnesium and zink composition as well as the phospholipid composition in the crude soybean oil, used in the experiments, is indicated in table 1A and 1B. The metal composition was measured by ICP-OES and individual phospholipids by LCMS.

TABLE 1A
Metal composition of crude oil measured by ICP-OES (mg/kg oil)

| | P | Ca | Mg | Zn |
|---|---|---|---|---|
| Crude oil 1 | 718 | 85 | 66 | 1 |
| Crude oil 2 | 649 | 60 | 57 | 1.2 |
| Crude oil 3 | 1030 | 110 | 93 | — |
| Crude oil 4 | 985 | 74 | 79 | 1.2 |
| Crude oil 5 | 684 | 82 | 62 | 1.0 |

TABLE 1B
Phospholipid composition of crude oil (mg/kg phosphorus).

| | Crude oil 1 | Crude oil 2 | Crude oil 5 |
|---|---|---|---|
| PA | 295 | 172 | 98 |
| PE | 125 | 225 | 191 |
| PI | 84 | 210 | 89 |
| PC | 229 | 283 | 185 |
| Total | 732 | 890 | 24 |

### Degumming Assay

Crude soybean oil (75 g) was initially acid/base pretreated (or not) to facilitate conversion of insoluble phospholipis salts into more hydratable forms and ensure an environment suitable for the enzyme. Acid/base pretreatment was done by acid addition of Ortho Phosphoric acid (85% solution) applied in amounts equal to 0.05% (100% pure Ortho Phosphoric acid) based on oil amount and mixing in ultrasonic bath (BRANSON 3510) for 5 min and incubation in rotator for 15 min followed by base neutralization with 4 M NaOH applied in equivalents (from 0.5 to 1.5) to pure Ortho Phosphoric acid in ultrasonic bath for 5 min. The enzyme reaction was conducted in low aqueous system (3% water total based on oil amount) in 100 ml centrifuge tubes, cylindrical, conical bottom. Samples were ultrasonic treated for 5 min, followed by incubation in a heated cabinet at selected temperature (from 50 to 60° C.) with stirring at 20 rpm for a selected incubation time (from 1 to 5 hours). To separate the mixture into an oil phase and a heavy water/gum phase the samples were centrifuged at 700 g at 85° C. for 15 min (Koehler Instruments, K600X2 oil centrifuge).

### a) Diglyceride Measurement

The HPLC-method (using DIONEX equipment and Lichrocart Si-60, 5 μm, Lichrosphere 250-4 mm, MERCK column) was based on the principle of the AOCS Official Method Cd 11 d-96 and quantifies the diglyceride content down to 0.1 wt %.

### b) Phosphorus/Phospholipid Measurement

The ICP-OES quantifies the phosphorus (P) content and other metals such as Ca, Mg, Zn down to 4 ppm with an accuracy of approximately ±1 ppm P.

### c) Quantitative Analysis of Phospholipids by LCMS/MS

Liquid Chromatography coupled to triple quadrupole mass spectrometer (LC/MS/MS) or coupled to quadrupole mass spectrometer time of flight (LC/TOF/MS) was used to quantify the individual phospholipids species: phosphatidylcholine (PC); Phosphatidylinositol (PI); Phosphatidylethanolamine (PE) and Phosphatidic acid (phosphatidate) (PA). The sensitivity of the assay goes down to less than 1 mg Phosphorus/kg oil for PC, PE and PI (ppm) and less than 10 mg Phosphorus/kg for PA. The oil sample was dissolved in chloroform. The extract was then analysed on LC-TOF-MS (or on LC-MS/MS if lower detection limits are needed) using following settings:

### LC-Settings

Eluent A: 50% Acetonitril, 50% Water, 0.15% formic acid
Eluent B: 100% Isopropionic acid, 0.15% formic acid
Run time: 26.9 min
Flow: 0.50 mL/min
Column temperature: 50° C.
Autosampler temp: 15-25° C.
Injection volume: 1 μL
Column type Material: Charged Surface Hybrid, length: 50 mm, size: 1.7 μm, ID: 2.1 mm

### MS-Settings

| TOF/MS | MS/MS (Xevo) |
|---|---|
| Capillary: 3.50 kV | Capillary: +3.50/−2.0 kV |
| Cone: 28 | Cone: Component specific |
| Extractor: 2 V | Extractor: 2.5 V |
| RF-lens: 0.5 V | RF-lens: |
| Source temp: 125° C. | Source temp: 150° C. |
| Desolvation temp: 500° C. | Desolvation temp: 500° C. |
| Cone gas flow: 30 L/hour | Cone gas flow: 30 L/hour |
| Desolvation gas flow: 850 L/hour | Desolvation gas flow: 850 L/hour |

The data was processed using MassLynx version 4.1 Software. In the below examples the method is just termed LCMS.

Example 3 to 6 below describes results obtained using the degumming assay of this example.

### Example 3

### Mature Polypeptide of SEQ ID NO: 2 and PURIFINE® PLC Blend

The mature polypeptide of SEQ ID NO: 2 applied in degumming assay at 60° C. in combination with PURIFINE® PLC at various enzyme dosages (enzyme protein per kg oil) applying oil 1. The diglyceride content after enzymatic degumming for 2, 3, 4 and 5 hrs were measured (oil pretreated with 0.05% phosphoric acid/1.5 eqv. NaOH) as well as the composition of individual phospholipids after 4 hours incubation measured by LCMS. The results are presented in table 2A and 2B.

TABLE 2A
Diglyceride increase (% w/w) after
enzyme incubation in oil 1 (hours)

| | Mature p.p. of SEQ | | | | |
|---|---|---|---|---|---|
| Purifine dosage | ID NO: 2 dosage | | | | |
| (mg EP/kg oil) | (mg EP/kg oil) | 2 | 3 | 4 | 5 |
| 10 | 20 | 0.58 | 0.83 | 0.95 | 1.18 |
| 10 | 10 | 0.55 | 0.69 | 0.74 | 0.91 |
| 4 | 20 | 0.47 | 0.67 | 0.80 | 0.98 |
| 4 | 10 | 0.46 | 0.65 | 0.79 | 0.92 |
| 4 | — | 0.33 | 0.59 | 0.61 | 0.63 |

TABLE 2B
Phospholipid composition measured by LCMS
after 4 hours enzyme incubation (mg/kg oil)

| | Mature p.p. of SEQ | | | | |
|---|---|---|---|---|---|
| Purifine dosage | ID NO: 2 dosage | | | | |
| (mg EP/kg oil) | (mg EP/kg oil) | PA | PE | PI | PC |
| 4 | 10 | ≦10* | 1.6 | 0 | 0 |

*Below PA detection limit

Degumming with the mature polypeptide of SEQ ID NO: 2 combined with PURIFINE® PLC results in significant diglyceride formation at 60° C. and converts up to 80% of the phospholipids at conditions tested (60° C., 5 hours). Conversion calculation is based on the assumption that 732 ppm P total measured by LC/MS is equal to 1.83 wt % phospholipid (Average PL Mw˜772 g/mol, Mw P-31 g/mol) equal to max 1.46% DG increase obtainable (80% of phospholipid molecule).

Results shows less than 10 mg P/kg oil originating from PI, PC, PE, PA in the degummed oil sample and confirms that the enzyme blend attacks all phospholipid species.

### Example 4

### Blend of Mature Polypeptide of SEQ ID NO: 2 and Mature Polypeptide of SEQ ID NO: 29

### C Rude Oil 2

The mature polypeptide of SEQ ID NO: 29 applied in degumming assay at 60 C in combination with the mature polypeptide of SEQ ID NO: 2 applying crude soybean oil 2 pretreated with 0.05% phosphoric acid/1.5 eqv. NaOH. The diglyceride content after enzymatic degumming for 2, 3, 4 and 5 hrs, were measured as well as phosphorous content after 5 hours degumming. The results are presented in table 3.

TABLE 3
Diglyceride increase (% w/w) after enzyme reaction (hours)

| | Bacterial | | | | | | |
|---|---|---|---|---|---|---|---|
| | dosage | Fungal | | | | | |
| | mature | mature p.p. | | | | | |
| Purifine | p.p. of SEQ | of SEQ ID | | | | | Phosphorous |
| dosage | ID NO: 29 | NO: 2 | | | | | after 5 hours |
| (mg EP/kg | (mg EP/kg | dosage (mg | | | | | measured by |
| oil) | oil) | EP/kg oil) | 2 | 3 | 4 | 5 | ICP (mg/kg) |
| 10 | | | 0.90 | 0.81 | 0.89 | 0.93 | 13 |
| | | 30 | 0.15 | 0.44 | 0.49 | 0.69 | 15 |
| | 10 | | 0.73 | 0.79 | 0.90 | 0.89 | 12 |
| | 10 | 30 | 0.87 | 1.22 | 1.32 | 1.41 | 10 |

Degumming with the PE; PC specific mature polypeptide of SEQ ID NO: 29 combined with mature polypeptide of SEQ ID NO: 2 result in a combined effect at 60° C. compared to individual solutions.

Full conversion is achieved after 4 hours based on the assumption that that 649 ppm P total measured by ICP is equal to 1.62 wt % phospholipid (Average PL Mw˜772 g/mol, Mw P-31 g/mol) equal to max 1.3% DG increase obtainable (80% of phospholipid molecule).

Phosphorous content in degummed oil was reduced to less than 10 mg/kg P total originating from PI, PC, PE, PA and confirms that the blend attack all phospholipid species.

### Example 5

### Mature Polypeptide of SEQ ID NO: 4 and Mature Polypeptide of SEQ ID NO: 29

The mature polypeptide of SEQ ID NO: 29 applied in degumming assay in combination with the mature polypeptide of SEQ ID NO: 4 applying crude soybean oil 3 without any acid/base pretreatment. The enzyme incubation is done at 60° C. (for 1^{st} hour) followed by incubation at 80° C. (for another 23 hours). The diglyceride content after enzymatic degumming for 1, 2, 4 and 24 hrs, were measured.

The results are presented in table 3.

TABLE 4
DG increase after enzyme incubation (hours)

| | 1 | 2 | 4 | 24 |
|---|---|---|---|---|
| Mature p.p. of SEQ ID NO: 29 in | 0.47 | 0.63 | 0.67 | 1.43 |
| B. subtilis (U1DW7) 4 mg EP | | | | |
| Mature p.p. of SEQ ID NO: 4 in T. reseii | 0.61 | 0.83 | 0.68 | 0.89 |
| (U1DW6) 10 mg EP | | | | |
| Mature p.p. of SEQ ID NO: 29 + Mature | 0.82 | 0.93 | 1.02 | 1.90 |
| p.p. of SEQ ID NO: 4 + 10 mg EP | | | | |

Degumming with the PE; PC specific mature polypeptide of SEQ ID NO: 29 combined with mature polypeptide of SEQ ID NO: 4 result in a combined effect compared to individual solutions. Almost full conversion (92.3%) is achieved after 24 hours based on the assumption that that 1030 ppm P total measured by ICP is equal to 2.58 wt % phospholipid (Average PL Mw˜772 g/mol, Mw P-31 g/mol) equal to max 2.06% DG increase obtainable (80% of phospholipid molecule).

### Example 6

### Triple of Mature Polypeptide of SEQ ID NO: 9+Mature Polypeptide of SEQ ID NO: 29+Mature Polypeptide of SEQ ID NO: 2

Mature polypeptide of SEQ ID NO: 2 applied in degumming assay at 60° C. in combination with mature polypeptide of SEQ ID NO: 9 and mature polypeptide of SEQ ID NO: 29 applying crude oil 4. The diglyceride content after enzymatic degumming for 2, 3, 5 and 24 hrs were measured (oil pretreated with 0.05% phosphoric acid/1.5 eqv. NaOH) as well as the total phosphorous content after 24 hours.

The results are presented in table 5A and 5B.

TABLE 5A
DG increase after enzyme incubation (hours) based on double
determinations with a coefficient of variation of max. 6.7%.

| Enzyme and dosing (mg EP/kg oil) | 2 | 3 | 5 | 24 |
|---|---|---|---|---|
| Mature p.p. of SEQ ID NO: 2 + | 0.66 | 0.68 | 0.74 | 1.04 |
| mature p.p. of SEQ ID NO: 29 | | | | |
| Mature p.p. of SEQ ID NO: 2 + | 0.69 | 0.76 | 0.84 | 1.17 |
| Mature p.p. of SEQ ID NO: 29 + | | | | |
| Mature p.p. of SEQ ID NO: 9 (10 + | | | | |
| 4 + 2) | | | | |

Degumming with a triple PLC solution result in an improved effect compared to a double PLC solutions. Phosphorous content in degummed oil (after 24 hours incubation) was reduced to less than 3 mg/kg P total originating from PI, PC, PE, PA and confirms that the enzyme blend used in the described assay result in full removal/conversion of all phospholipid species.

### Example 7

### Triple of Mature Polypeptide of SEQ ID NO: 9+Mature Polypeptide of SEQ ID NO: 29+Mature Polypeptide of SEQ ID NO: 7

Mature polypeptide of SEQ ID NO: 7 applied in degumming assay at 60° C. in combination with mature polypeptide of SEQ ID NO: 9 and mature polypeptide of SEQ ID NO: 29 applying crude oil 5. The diglyceride content after enzymatic degumming for 2, 3, and 5 hrs were measured (oil pretreated with 0.05% phosphoric acid/1.5 eqv. NaOH) as well as the total phosphorous content after 5 hours.

TABLE 6
Increase of diglyceride after enzyme treatment measured by
HPLC-Corona Veo and phosphorous content measured by ICP
after oil pre-treatment with of 0.05% phosphoric acid/1.5 eqv. NaOH.

| | | | | | | Total |
|---|---|---|---|---|---|---|
| | | | DG increase | | | P after 5 h enz |
| | | | after enzyme | | | reaction |
| | Enz dosing | Water | reaction (hours) | | | mg/kg |
| Enzyme | (mg EP/kg oil) | dosing | 2 | 3 | 5 | oil = ppm |
| No enz. | — | 3% water | 0.00 | 0.00 | 0.02 | 43 |
| Seq 2 + 29 + 9 | 10 + 4 + 2 | | 0.35 | 0.40 | 0.56 | 35 |
| Seq 5 + 29 + 9 | 10 + 4 + 2 | | 0.29 | 0.34 | 0.51 | 30 |
| Seq 2 + 29 + 9 | 30 + 10 + 4 | | 0.36 | 0.44 | 0.65 | 26 |
| Seq 5 + 29 + 9 | 30 + 10 + 4 | | 0.22 | 0.28 | 0.44 | 31 |
| No enz. | | 5% water | 0.00 | 0.00 | 0.03 | 18 |
| Seq 5 + 29 + 9 | 10 + 4 + 2 | | 0.50 | 0.55 | 0.73 | 27 |
| Seq 5 + 29 + 9 | 30 + 10 + 4 | | 0.35 | 0.46 | 0.68 | 19 |

Degumming with SEQ ID NO: 5+29+9 triple PLC solution result in significant diglyceride formation (up to 0.76 wt % DG) after 2-5 hours reaction. Improved performance of SEQ ID NO: 5+29+9 in terms of extra diglyceride formation was observed when water content was increased from 3 to 5% based on oil amount.

### Example 8

### Mature Polypeptide of SEQ ID NO: 5 and Mature Polypeptide of SEQ ID NO: 29

The mature polypeptide of SEQ ID NO: 29 applied in degumming assay in combination with the mature polypeptide of SEQ ID NO: 5 applying crude soybean oil 5 after oil pre-treatment with 355 ppm phosphoric acid+1.5 eqv NaOH. The enzyme incubation is done at 60° C. for 5 hours.

| | | | | | | Total P after |
|---|---|---|---|---|---|---|
| | Enz | | DG increase | | | 5 h enz |
| | dosing | | after enzyme | | | reaction |
| | (mg EP/ | Water | reaction (hours) | | | mg/kg |
| Enzyme | kg oil) | dosing | 2 | 3 | 5 | oil = ppm |
| no enz-blank | — | 3% | — | — | — | 70 |
| control | | | | | | |
| Seq ID 5 | 10 | | 0.12 | 0.07 | 0.11 | 76 |
| Seq ID 5 + 29 | 10 + 4 | | 0.19 | 0.17 | 0.18 | 65 |
| Seq ID 5 + 29 | 30 + 10 | | 0.27 | 0.24 | 0.33 | 63 |
| no enz-blank | — | 5% | — | — | — | 62 |
| control | | | | | | |
| Seq ID 5 | 10 | | 0.10 | 0.13 | 0.25 | 65 |
| Seq ID 5 + 29 | 10 + 4 | | 0.42 | 0.51 | 0.59 | 63 |
| Seq ID 5 + 29 | 30 + 10 | | 0.55 | 0.59 | 0.79 | 63 |

Degumming with a blend PLC solution of SEQ ID 5+SEQ ID 29 results in an improved performance in terms of diglyceride formation when compared to single component PLC solution SEQ ID 5.

### Example 9

### Mature Polypeptide of SEQ ID NO: 4 and Mature Polypeptide of SEQ ID NO: 29

The mature polypeptide of SEQ ID NO: 29 applied in degumming assay in combination with the mature polypeptide of SEQ ID NO: 4 applying crude soybean oil after oil pre-treatment with 650 ppm citric acid+1.5 eqv NaOH. Total water content is 3% w/w. The enzyme incubation is done at 60° C. or 70° C. for 22 hours.

| | Enz dosing | Incubation | DG increase after enzyme | | |
|---|---|---|---|---|---|
| | (mg EP/kg | temperature | reaction (hours) | | |
| Enzyme | oil) | ° C. | 2 | 5 | 22 |
| Seq ID 29 | 10 | 60 | 0.24 | 0.25 | 0.28 |
| Seq ID X + 29 | 30 + 10 | 60 | 0.44 | 0.61 | 0.99 |
| Seq ID 29 | 10 | 70 | 0.12 | 0.19 | 0.19 |
| Seq ID X + 29 | 30 + 10 | 70 | 0.42 | 0.54 | 0.64 |

Degumming with a blend PLC solution of SEQ ID NO: 4+SEQ ID NO: 29 results in an improved performance in terms of diglyceride formation when compared to single component PLC solution SEQ ID NO: 29.

### Example 10

### Mature Polypeptide of SEQ ID NO: 2 and Mature Polypeptide of SEQ ID NO: 29

The mature polypeptide of SEQ ID NO: 29 applied in degumming assay in combination with the mature polypeptide of SEQ ID NO: 2 applying crude soybean oil after oil pre-treatment with 650 ppm citric acid+1.5 eqv NaOH. Total water content is 3% w/w. The enzyme incubation is done at 60° C. for 5 hours.

| | | DG increase after enzyme | | |
|---|---|---|---|---|
| | Enz dosing | reaction (hours) | | |
| Enzyme | (mg EP/kg oil) | 2 | 3 | 5 |
| Seq ID 29 | 10 | 0.17 | 0.17 | 0.19 |
| Seq ID 2 | 30 | 0.26 | 0.31 | 0.41 |
| Seq ID 2 + 29 | 10 + 30 | 0.45 | 0.48 | 0.63 |
| Blank | 0 | 0.02 | 0.03 | 0.00 |

Degumming with a blend PLC solution of SEQ ID NO: 2+SEQ ID 29 results in an improved performance in terms of diglyceride formation when compared to single component PLC solutions SEQ ID NO: 29 or SEQ ID NO: 2.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A composition comprising a plurality of polypeptides having phospholipase C activity, wherein
at least one of said polypeptides having phospholipase C activity is a polypeptide derived from a fungus; and
at least one of said polypeptides having phospholipase C activity is a polypeptide derived from a bacterium.

2. The composition according to claim 1, wherein one or more of said polypeptides derived from a fungus has activity towards phosphatidylcholine (PC), phosphatidylethanoamine (PE), phosphatidic acid (PA), and phosphatidyl inositol (PI).

3. The composition according to claim 1, wherein one or more of said polypeptides derived from a bacterium has activity towards phosphatidyl inositol (PI).

4. The composition according to claim 1, wherein one or more of said polypeptides derived from a bacterium has/have activity towards phosphatidylcholine (PC) and phosphatidylethanoamine (PE).

5. The composition according to claim 1, comprising a polypeptide, which has phospholipase C activity, is derived from a fungus and is selected from the group consisting of:
(a) a polypeptide having at least 60% sequence identity to the mature polypeptide of any one of SEQ ID NO: 2, SEQ ID NO: 4, and SEQ ID NO: 7;
(b) a polypeptide encoded by a polynucleotide that hybridizes under medium high stringency conditions with (i) the mature polypeptide coding sequence of any one of SEQ ID NO: 1, SEQ ID NO: 3 and SEQ ID NO: 5 (ii) the genomic DNA sequence comprising the mature polypeptide coding sequence of any one of SEQ ID NO: 1, SEQ ID NO: 3 and SEQ ID NO: 5, (iii) the cDNA sequence of SEQ ID NO: 5 (SEQ ID NO: 6) or (iv) the full-length complementary strand of (i), (ii) or (iii);
(c) a polypeptide encoded by a polynucleotide having at least 60% sequence identity to the mature polypeptide coding sequence of anyone of SEQ ID NO: 1, SEQ ID NO: 3 and SEQ ID NO: 5;
(d) a variant of the mature polypeptide of any one of SEQ ID NO: 2, SEQ ID NO: 4, and SEQ ID NO: 7 comprising a substitution, deletion, and/or insertion at one or more positions; and
(e) a fragment of the polypeptide of (a), (b), (c), or (d).

6. The composition according to claim 1, comprising a polypeptide, which has phospholipase C activity, is derived from a bacterium and is selected from the group consisting of:
(a) a polypeptide having at least 60% sequence identity to the mature polypeptide of any one of SEQ ID NO: 9, SEQ ID NO: 12, SEQ ID NO: 15, SEQ ID NO: 18, SEQ ID NO: 21, SEQ ID NO: 24, SEQ ID NO: 26, SEQ ID NO: 29, SEQ ID NO: 31, SEQ ID NO: 34, SEQ ID NO: 36, SEQ ID NO: 39, SEQ ID NO: 41, SEQ ID NO: 43 and SEQ ID NO: 45;
(b) a polypeptide encoded by a polynucleotide that hybridizes under medium high stringency conditions with (i) the mature polypeptide coding sequence of any one of SEQ ID NO: 8, SEQ ID NO: 11, SEQ ID NO: 14, SEQ ID NO: 17, SEQ ID NO: 20, SEQ ID NO: 23 SEQ ID NO: 25, SEQ ID NO: 28, SEQ ID NO: 30, SEQ ID NO: 33, SEQ ID NO: 35, SEQ ID NO: 38, SEQ ID NO: 40, SEQ ID NO: 42 and SEQ ID NO: 44 (ii) the genomic DNA sequence comprising the mature polypeptide coding sequence of any one of SEQ ID NO: 8, SEQ ID NO: 11, SEQ ID NO: 14, SEQ ID NO: 17, SEQ ID NO: 20, SEQ ID NO: 23, SEQ ID NO: 25, SEQ ID NO: 28, SEQ ID NO: 30, SEQ ID NO: 33, SEQ ID NO: 35, SEQ ID NO: 38, SEQ ID NO: 40, SEQ ID NO: 42 and SEQ ID NO: 44 or (iii) the full-length complementary strand of (i) or (ii);
(c) a polypeptide encoded by a polynucleotide having at least 60% sequence identity to the mature polypeptide coding sequence of any one of SEQ ID NO: 8, SEQ ID NO: 11, SEQ ID NO: 14, SEQ ID NO: 17, SEQ ID NO: 20, SEQ ID NO: 23, SEQ ID NO: 25, SEQ ID NO: 28, SEQ ID NO: 30, SEQ ID NO: 33, SEQ ID NO: 35, SEQ ID NO: 38, SEQ ID NO: 40, SEQ ID NO: 42 and SEQ ID NO: 44;
(d) a variant of the mature polypeptide of any one of SEQ ID NO: 9, SEQ ID NO: 12, SEQ ID NO: 15, SEQ ID NO: 18, SEQ ID NO: 21, SEQ ID NO: 24, SEQ ID NO: 26, SEQ ID NO: 29, SEQ ID NO: 31, SEQ ID NO: 34, SEQ ID NO: 36, SEQ ID NO: 39, SEQ ID NO: 41, SEQ ID NO: 43 and SEQ ID NO: 45, comprising a substitution, deletion, and/or insertion at one or more positions; and
(e) a fragment of the polypeptide of (a), (b), (c), or (d).

7. The composition according to claim 1, comprising a polypeptide, which has phospholipase C activity and is selected from the group consisting of:
(a) a polypeptide having at least 60% sequence identity to the mature polypeptide of SEQ ID NO: 41;
(b) a polypeptide encoded by a polynucleotide that hybridizes under medium high stringency conditions with (i) the mature polypeptide coding sequence of SEQ ID NO: 40 (ii) the genomic DNA sequence comprising the mature polypeptide coding sequence of SEQ ID NO: 40, or (iii) the full-length complementary strand of (i) or (ii);
(c) a polypeptide encoded by a polynucleotide having at least 60% sequence identity to the mature polypeptide coding sequence of SEQ ID NO: 40;
(d) a variant of the mature polypeptide of SEQ ID NO: 41, comprising a substitution, deletion, and/or insertion at one or more positions; and
(e) a fragment of the polypeptide of (a), (b), (c), or (d).

8. The composition according to claim 1, wherein said plurality of polypeptides having phospholipase C activity is selected from the group consisting of:
one polypeptide which is derived from a fungus and one polypeptide which is derived from a bacterium;
one polypeptide which is derived from a fungus and two polypeptides which are each derived from a bacterium;
one polypeptide which is derived from a fungus and three polypeptides which are derived from a bacterium;
two polypeptides which are each derived from a fungus and one polypeptide which is derived from a bacterium;
two polypeptides which are each derived from a fungus and two polypeptides which are each derived from a bacterium;
two polypeptides which are each derived from a fungus and three polypeptides which are each derived from a bacterium;
three polypeptides which are each derived from a fungus and one polypeptide which is derived from a bacterium;
three polypeptides which are each derived from a fungus and two polypeptides which are each derived from a bacterium;
three polypeptides which are each derived from a fungus and three polypeptides which are each derived from a bacterium.

9. The composition of claim 1, for use in
i) degumming of an aqueous carbohydrate solution or slurry, in degumming of oil, or in a process comprising hydrolysis of phospholipids in the gum fraction from water degumming to release entrapped triglyceride oil,
ii) a process comprising hydrolysis of phospholipids to obtain improved phospholipid emulsifiers, in particular wherein said phospholipid is lecithin,
iii) a process for improving the filterability of an aqueous solution or slurry of carbohydrate origin which contains phospholipid,
iv) a process for the extraction of oil,
v) a process for the production of an animal feed product,
vi) a process for the production of a biofuel, e.g. a biodiesel,
vii) a process for the production of a detergent product, and/or
viii) a process for making a baked product, comprising adding the phospholipase to a dough, and baking the dough to make the baked product.

10. A process for hydrolysing a phospholipid or lysophospholipid, comprising contacting said phospholipid or lysophospholipid with a plurality of polypeptides having phospholipase C activity, wherein
at least one of said polypeptides having phospholipase C activity is a polypeptide derived from a fungus; and
at least one of said polypeptides having phospholipase C activity is a polypeptide derived from a bacterium.

11. The process according to claim 10, wherein said plurality of polypeptides having phospholipase C activity is selected from the group consisting of:
one polypeptide which is derived from a fungus and one polypeptide which is derived from a bacterium;
one polypeptide which is derived from a fungus and two polypeptides which are each derived from a bacterium;
one polypeptide which is derived from a fungus and three polypeptides which are each derived from a bacterium;
two polypeptides which are each derived from a fungus and one polypeptide which is derived from a bacterium;
two polypeptides which are each derived from a fungus and two polypeptides which are derived from a bacterium;
two polypeptides which are each derived from a fungus and three polypeptides which are derived from a bacterium;
three polypeptides which are each derived from a fungus and one polypeptide which is derived from a bacterium;
three polypeptides which are each derived from a fungus and two polypeptides which are each derived from a bacterium;
three polypeptides which are each derived from a fungus and three polypeptides which are each derived from a bacterium.

12. The process according to claim 10, wherein said polypeptide, which has phospholipase C activity and is derived from a fungus is selected from the group consisting of:
(a) a polypeptide having at least 60% sequence identity to the mature polypeptide of any one of SEQ ID NO: 2, SEQ ID NO: 4, and SEQ ID NO: 7;
(b) a polypeptide encoded by a polynucleotide that hybridizes under medium high stringency conditions with (i) the mature polypeptide coding sequence of any one of SEQ ID NO: 1, SEQ ID NO: 3 and SEQ ID NO: 5 (ii) the genomic DNA sequence comprising the mature polypeptide coding sequence of any one of SEQ ID NO: 1, SEQ ID NO: 3 and SEQ ID NO: 5, (iii) the cDNA sequence of SEQ ID NO: 5 (SEQ ID NO: 6) or (iv) the full-length complementary strand of (i), (ii) or (iii);
(c) a polypeptide encoded by a polynucleotide having at least 60% sequence identity to the mature polypeptide coding sequence of anyone of SEQ ID NO: 1, SEQ ID NO: 3 and SEQ ID NO: 5;
(d) a variant of the mature polypeptide of any one of SEQ ID NO: 2, SEQ ID NO: 4, and SEQ ID NO: 7 comprising a substitution, deletion, and/or insertion at one or more positions; and
(e) a fragment of the polypeptide of (a), (b), (c), or (d).

13. The process according to claim 10, comprising contacting said phospholipid or lysophospholipid with a polypeptide, which has phospholipase C activity and is selected from the group consisting of:
(a) a polypeptide having at least 60% sequence identity to the mature polypeptide of SEQ ID NO: 41;
(b) a polypeptide encoded by a polynucleotide that hybridizes under medium high stringency conditions with (i) the mature polypeptide coding sequence of SEQ ID NO: 40 (ii) the genomic DNA sequence comprising the mature polypeptide coding sequence of SEQ ID NO: 40, or (iii) the full-length complementary strand of (i) or (ii);
(c) a polypeptide encoded by a polynucleotide having at least 60% sequence identity to the mature polypeptide coding sequence of SEQ ID NO: 40;
(d) a variant of the mature polypeptide of SEQ ID NO: 41, comprising a substitution, deletion, and/or insertion at one or more positions; and
(e) a fragment of the polypeptide of (a), (b), (c), or (d).

14. The process according to claim 10, comprising contacting said phospholipid or lysophospholipid with a polypeptide, which has phospholipase C activity, and is selected from the group consisting of:
(a) a polypeptide having at least 60% sequence identity to the mature polypeptide of any one of SEQ ID NO: 9, SEQ ID NO: 12, SEQ ID NO: 15, SEQ ID NO: 18, SEQ ID NO: 21, SEQ ID NO: 24, SEQ ID NO: 26, SEQ ID NO: 29, SEQ ID NO: 31, SEQ ID NO: 34, SEQ ID NO: 36, SEQ ID NO: 39, SEQ ID NO: 41, and SEQ ID NO: 43 and SEQ ID NO: 45;
(b) a polypeptide encoded by a polynucleotide that hybridizes under medium high stringency conditions with (i) the mature polypeptide coding sequence of any one of SEQ ID NO: 8, SEQ ID NO: 11, SEQ ID NO: 14, SEQ ID NO: 17, SEQ ID NO: 20, SEQ ID NO: 23 SEQ ID NO: 25, SEQ ID NO: 28, SEQ ID NO: 30, SEQ ID NO: 33, SEQ ID NO: 35, SEQ ID NO: 38, SEQ ID NO: 40, SEQ ID NO: 42 and SEQ ID NO: 44 (ii) the genomic DNA sequence comprising the mature polypeptide coding sequence of any one of SEQ ID NO: 8, SEQ ID NO: 11, SEQ ID NO: 14, SEQ ID NO: 17, SEQ ID NO: 20, SEQ ID NO: 23, SEQ ID NO: 25, SEQ ID NO: 28, SEQ ID NO: 30, SEQ ID NO: 33, SEQ ID NO: 35, SEQ ID NO: 38, SEQ ID NO: 40, SEQ ID NO: 42 and SEQ ID NO: 44 or (iii) the full-length complementary strand of (i) or (ii);
(c) a polypeptide encoded by a polynucleotide having at least 60% sequence identity to the mature polypeptide coding sequence of any one of SEQ ID NO: 8, SEQ ID NO: 11, SEQ ID NO: 14, SEQ ID NO: 17, SEQ ID NO: 20, SEQ ID NO: 23, SEQ ID NO: 25, SEQ ID NO: 28, SEQ ID NO: 30, SEQ ID NO: 33, SEQ ID NO: 35, SEQ ID NO: 38, SEQ ID NO: 40, SEQ ID NO: 42 and SEQ ID NO: 44;
(d) a variant of the mature polypeptide of any one of SEQ ID NO: 9, SEQ ID NO: 12, SEQ ID NO: 15, SEQ ID NO: 18, SEQ ID NO: 21, SEQ ID NO: 24, SEQ ID NO: 26, SEQ ID NO: 29, SEQ ID NO: 31, SEQ ID NO: 34, SEQ ID NO: 36, SEQ ID NO: 39, SEQ ID NO: 41 SEQ ID NO: 43 and SEQ ID NO: 45, comprising a substitution, deletion, and/or insertion at one or more positions; and
(e) a fragment of the polypeptide of (a), (b), (c), or (d).
